(19) United States
(12) Patent Application Publication
(10) Pub. No.: US 2025/0297245 A1
(43) Pub. Date: Sep. 25, 2025

(54) COMPOSITIONS AND METHODS FOR SINGLE-CELL RNA SEQUENCING

(71) Applicants: Dana-Farber Cancer Institute, Inc., Boston, MA (US); The General Hospital Corporation, Boston, MA (US); The Brigham and Women’s Hospital, Inc., Boston, MA (US)

(72) Inventors: Romanos Sklavenitis Pistofidis, Boston, MA (US); Gad Getz, Boston, MA (US); Irene Ghobrial, Boston, MA (US); Peter Van Galen, Boston, MA (US)

(73) Assignees: Dana-Farber Cancer Institute, Inc., Boston, MA (US); The General Hospital Corporation, Boston, MA (US); The Brigham and Women’s Hospital, Inc., Boston, MA (US)

(21) Appl. No.: 19/087,123

(22) Filed: Mar. 21, 2025

Related U.S. Application Data
(60) Provisional application No. 63/568,979, filed on Mar. 22, 2024.

(51) Int. Cl. C12N 15/10

(52) U.S. Cl. CPC C12N 15/1093

(57) ABSTRACT

Methods for single-cell sequencing of mitochondrial RNA are described. In some embodiments, the methods further involve the identification of malignant cells and/or characterization of tumor subclones in a biological sample.

### CROSS-REFERENCE TO RELATED APPLICATION

This application is a U.S. utility application that claims priority to and the benefit of U.S. Provisional Application No. 63/568,979, filed Mar. 22, 2024, the entire contents of which are incorporated herein by reference.

### SEQUENCE LISTING

The instant application contains a Sequence Listing which has been submitted electronically in XML format and is hereby incorporated by reference in its entirety. Said XML copy, created on Apr. 29, 2025, is named 167741-052701_US_SL.xml and is 297,849 bytes in size.

### BACKGROUND

Single-cell RNA sequencing enables the parallel profiling of thousands of cells and dozens of cell types from a single sample; however, determining which cells may be malignant and resolving tumor subclones remains challenging. Nevertheless, the ability to identify malignant cells (i.e., clones) and tumor subclones in single-cell RNA sequencing data is important for the detection, characterization and treatment of disease. With the exception of B cell and T cell malignancies, which express unique barcode-like sequences in the form of the B cell receptor (BCR) and T cell receptor (TCR) respectively, malignant cells may be indistinguishable from normal cells in single-cell RNA sequencing assays. The ability to identify tumor subclones is important for understanding disease progression, understanding the impact of disease progression on the tumor cell transcriptome, and selecting therapies for a heterologous population of tumor cells. Many current methods for identifying tumor clones or subclones rely heavily on copy number variant (CNV) inference tools and are, therefore, limited to clones or subclones with CNVs. Alternatively, current methods for identifying clones or subclones of cells using single-cell RNA sequencing data rely on identification of somatic mutations, which can be challenging to detect or infer. In particular, single nucleotide variants (SNVs) cannot be detected in all positive cells due to allelic dropout and/or insufficient coverage and, although genes or amplicons of interest can be further amplified in a particular tumor sample to enhance the probability of detection, such approaches cannot be generalized across samples and require prior knowledge of the mutational landscape of each individual tumor.

Mitochondrial DNA mutations are a powerful tool to identify clones of cells (malignant or not) in single-cell RNA sequencing samples. In particular, mitochondrial DNA mutations are a powerful and generalizable tool to identify clones of cells (malignant or not) in single-cell RNA and assay for transposase-accessible chromatin (ATAC) sequencing data. The mitochondrial genome is relatively small (˜16.6kb), which allows for the development of one-size-fits-all targeted amplification panels that can be applied to any sample. Furthermore, each cell typically contains 100 to 100,000 mitochondria and, therefore, high counts of copies of mitochondrial DNA (mtDNA) compared to the only 2 copies of normal nuclear DNA in diploid cells, thereby facilitating more ready detection of SNVs in mitochondrial DNA than in nuclear DNA. Notably, approximately 1-15% of the transcriptome in mononuclear cells comprises mtRNA. Also, the mitochondrial genome is more prone to mutations having a mutation rate that is 10-100-fold higher than nuclear DNA, which means that a given clone may carry more than a single mutation, which has the potential to further improve the probability of correct clone assignments.

RNA within a single cell may be sequenced from the 3′-end (i.e., 3′-end sequencing) or the 5′-end (5′-end sequencing); however, no methods are presently available for 5′-end single-cell sequencing of mitochondrial RNA that enables lineage tracing.

### SUMMARY

As described below, the present disclosure features, among other things, methods for single-cell sequencing of mitochondrial RNA (e.g., 5′ end single cell mitochondrial sequencing). In some embodiments, the methods of the disclosure further involve the identification of malignant cells and/or characterization of tumor subclones in a biological sample.

In one aspect, the disclosure features a method for preparation of a mitochondrial cDNA sequencing library. The method involves a) preparing a cDNA library derived from a cell containing a mitochondrion, wherein the cDNA library contains polynucleotides. Each polynucleotide contains in order from 5′ to 3′ the nucleotide sequence CTACACGACGCTCTTCCGATCT (SEQ ID NO: 1), or a variant thereof with up to 5 nucleotide alterations, and a full-length cDNA polynucleotide. The 5′ end of the cDNA polynucleotide corresponds to the 5′ end of an mRNA molecule derived from the cell. The method also involves b) contacting the cDNA library with a DNA polymerase, a first forward primer, and a first reverse primer under conditions sufficient to amplify polynucleotides present in the cDNA library in a first PCR reaction. The first forward primer contains the sequence ACACTCTTTCCCTACACGACGCTCTTCCGATCT (SEQ ID NO: 2), or a variant thereof with up to 5 or 10 nucleotide alterations. The first reverse primer contains in order from 5′ to 3′ the nucleotide sequence CACCCGAGAATTCCA (SEQ ID NO: 3), or a variant thereof with up to 5 nucleotide alterations, and a sequence complementary to a mitochondrial cDNA polynucleotide. The first PCR reaction yields first PCR amplicons. The method further involves c) contacting the first PCR amplicons with a DNA polymerase, a second forward primer, and a second reverse primer under conditions sufficient to amplify the first PCR amplicons in a second PCR reaction. The second forward primer for the second PCR reaction contains in order from 5′ to 3′ the nucleotide sequence AATGATACGGCGACCACCGAGATCTACAC (SEQ ID NO: 4) or a variant thereof with up to 5 nucleotide alterations, and the nucleotide sequence ACACTCTTTCC (SEQ ID NO: 5), or a variant thereof with up to 5 nucleotide alterations. The second reverse primer contains in order from 5′ end to 3′ end the nucleotide sequence CAAGCAGAAGACGGCATACGAGAT (SEQ ID NO: 6), or a variant thereof with up to 5 nucleotide alterations, and the nucleotide sequence GTGACTGGAGTTCCTTGGCACCCGAGAATTCCA (SEQ ID NO: 7), or a variant thereof with up to 5 nucleotide alterations. The method results in preparation of the mitochondrial cDNA sequencing library.

In another aspect, the disclosure features a method for preparation of a mitochondrial cDNA sequencing library. The method involves a) preparing a cDNA library derived from single cells each comprising a mitochondrion. The cDNA library is prepared using a Gel Beads-in-emulsion (GEM) method. The cDNA library contains polynucleotides, wherein each polynucleotide contains in order from 5′ to 3′ the nucleotide sequence CTACACGACGCTCTTCCGATCT (SEQ ID NO: 1), or a variant thereof with up to 5 nucleotide alterations, a barcode identifying a cell from which a full-length cDNA polynucleotide is derived, a unique molecular identifier identifying an mRNA molecule from which the full-length cDNA polynucleotide is derived, the nucleotide sequence TTTCTTATATGGG (SEQ ID NO: 8), or a variant thereof with up to 5 nucleotide alterations, the full-length cDNA polynucleotide, and the nucleotide sequence GTACTCTGCGTTGATACCACTGCTT (SEQ ID NO: 9), or a variant thereof with up to 5 nucleotide alterations. The 5′ end of the cDNA polynucleotide corresponds to the 5′ end of an mRNA molecule derived from the cell. The method also involves b) completing twelve independent first PCR reactions. Each of the twelve first PCR reactions involves contacting the cDNA library with a DNA polymerase, a first forward primer containing the sequence ACACTCTTTCCCTACACGACGCTCTTCCGATCT (SEQ ID NO: 2), and a unique set of first reverse primers containing an RNA sequencing mixture of Table 2. The ratio of the first forward primer to the first reverse primer in each first PCR reaction is from about 1:2 to about 1:3. Each first PCR reaction involves about 13 extension cycles and an annealing temperature of about 60° C. The first PCR reaction yields twelve sets of first PCR amplicons. The method further involves c) pooling the first PCR amplicons from each of the twelve first PCR reactions at the following volumetric ratios to yield pooled first PCR amplicons: 40 volumetric units of each of the first PCR amplicons corresponding to primer mixes 1-9, 12 volumetric units of the first PCR amplicon mixture corresponding to primer mix 10, 8 volumetric units of the first PCR amplicon mixture corresponding to each of primer mixes 11 and 12. The method also involves d) cleaning the pooled first PCR amplicons using solid-phase reverse immobilization (SPRI) beads. The cleaning involves eluting the PCR amplicons from the SPRI beads using about 100 μL of a nuclease-free solution containing water to yield eluted amplicons. The method further involves e) contacting the eluted amplicons with a DNA polymerase, a second forward primer, and a second reverse primer under conditions sufficient to amplify the first PCR amplicons in a second PCR reaction. The second forward primer for the second PCR reaction contains in order from 5′ to 3′ the nucleotide sequence AATGATACGGCGACCACCGAGATCTACAC (SEQ ID NO: 4) or a variant thereof with up to 5 nucleotide alterations, a first indexing sequence identifying the mitochondrial cDNA sequencing library, and the nucleotide sequence ACACTCTTTCC (SEQ ID NO: 5), or a variant thereof with up to 5 nucleotide alterations. The second reverse primer contains in order from 5′ end to 3′ end the nucleotide sequence CAAGCAGAAGACGGCATACGAGAT (SEQ ID NO: 6), or a variant thereof with up to 5 nucleotide alterations, a second indexing sequence identifying the mitochondrial cDNA sequencing library, and the nucleotide sequence

GTGACTGGAGTTCCTTGGCACCCGAGAATTCCA (SEQ ID NO: 7), or a variant thereof with up to 5 nucleotide alterations. The ratio of the second forward primer to the second reverse primer in each second PCR reaction is from about 1:4 to about 1:6. The second PCR reaction involves an annealing temperature of about 54° C. The method results in preparation of the mitochondrial cDNA sequencing library.

In another aspect, the disclosure features a method for sequencing mitochondrial cDNA. The method involves sequencing the mitochondrial cDNA sequencing library prepared according to the method of any aspect of the disclosure, or embodiments thereof.

In another aspect, the disclosure features a method for detecting a clone of cells. The method involves sequencing mitochondrial cDNA according to the method of any aspect of the disclosure, or embodiments thereof, to yield sequence data and analyzing the sequence data to identify single nucleotide variants (SNVs) and/or copy number variants (CNVs) in the sequence data. The mitochondrial mRNA is from a biological sample containing cells and obtained from a subject. Identified SNVs and/or CNVs are used to detect a clone of cells in the biological sample.

In another aspect, the disclosure features a method for characterizing clone cells. The method involves sequencing mitochondrial cDNA according to the method of any aspect of the disclosure, or embodiments thereof, to yield sequence data and analyzing the sequence data to identify single nucleotide variants (SNVs) and/or copy number variants (CNVs) in the sequence data. The mitochondrial mRNA is from a biological sample obtained from a subject containing the clone cells. The biological sample contains the clone cells.

In another aspect, the disclosure features a method for monitoring a therapy. The method involves characterizing a neoplasia in a subject at at least two time points by sequencing mitochondrial cDNA according to the method of any aspect of the disclosure, or embodiments thereof, to yield sequence data and analyzing the sequence data to identify single nucleotide variants (SNVs) and/or copy number variants (CNVs) in the sequence data. The mitochondrial mRNA is from a biological sample obtained from a subject having the neoplasia. The biological sample contains neoplastic cells. The subject has been administered a treatment for the neoplasia.

In another aspect, the disclosure features a method for characterizing progression of a neoplasia in a subject. The method involves characterizing a neoplasia in the subject at at least two time points by sequencing mitochondrial cDNA according to the method of any aspect of the disclosure, or embodiments thereof, to yield sequence data and analyzing the sequence data to identify single nucleotide variants (SNVs) and/or copy number variants (CNVs) in the sequence data, where the mitochondrial mRNA is from a biological sample obtained from a subject having the neoplasia, and where the biological sample comprises neoplastic cells.

In another aspect, the disclosure features a primer selected from the full-length RNA sequencing primers listed in Table 2.

In another aspect, the disclosure features a set of primers for use in preparing a mitochondrial cDNA sequencing library. The set of primers contain a set of primers corresponding to an RNA sequencing primer mixture listed in Table 2.

In another aspect, the disclosure features a set of compositions, where each composition contains a unique set of primers corresponding to an RNA Sequencing Primer Mixture listed in Table 2.

In another aspect, the disclosure features a kit suitable for use in the method of any aspect of the disclosure, or embodiments thereof. The kit contains a) a primer containing the nucleotide sequence ACACTCTTTCCCTACACGACGCTCTTCCGATCT (SEQ ID NO: 2), or a variant thereof with up to 5 nucleotide alterations, b) a primer containing in order from 5′ to 3′ the nucleotide sequence AATGATACGGCGACCACCGAGATCTACAC (SEQ ID NO: 4) or a variant thereof with up to 5 nucleotide alterations, a first indexing primer, and the nucleotide sequence ACACTCTTTCC (SEQ ID NO: 5), or a variant thereof with up to 5 nucleotide alterations, c) a primer containing in order from 5′ to 3′ the nucleotide sequence CACCCGAGAATTCCA (SEQ ID NO: 3), or a variant thereof with up to 5 nucleotide alterations, and a sequence complementary to a mitochondrial cDNA polynucleotide; and/or d) a primer containing in order from 5′ to 3′ the nucleotide sequence CAAGCAGAAGACGGCATACGAGAT (SEQ ID NO: 6), or a variant thereof with up to 5nucleotide alterations, a second indexing primer, and the nucleotide sequence GTGACTGGAGTTCCTTGGCACCCGAGAATTCCA (SEQ ID NO: 7), or a variant thereof with up to 5 nucleotide alterations.

In another aspect, the disclosure features a method for preparation of a mitochondrial cDNA sequencing library. The method involves a) preparing a cDNA library derived from a cell containing a mitochondrion, where the cDNA library contains polynucleotides. Each polynucleotide contains in order from 5′ to 3′ a first partial Read 1 sequence, and a full-length cDNA polynucleotide, where the 5′ end of the cDNA polynucleotide corresponds to the 5′ end of an mRNA molecule derived from the cell. The method also involves b) contacting the cDNA library with a DNA polymerase, a first forward primer, and a first reverse primer under conditions sufficient to amplify polynucleotides present in the cDNA library in a first PCR reaction. The first forward primer contains a Read 1 sequence containing from 5′ to 3′, a second partial Read 1 sequence and the first partial Read 1 sequence. The first reverse primer contains in order from 5′ to 3′ a first partial Read 2 sequence and a sequence complementary to a mitochondrial cDNA polynucleotide. The first PCR reaction yields first PCR amplicons. The method further involves c) contacting the first PCR amplicons with a DNA polymerase, a second forward primer, and a second reverse primer under conditions sufficient to amplify the first PCR amplicons in a second PCR reaction. The second forward primer for the second PCR reaction contains in order from 5′ to 3′ a P5 sequence and the second partial Read 1 sequence. The second reverse primer contains in order from 5′ end to 3′ end a P7 sequence, and a Read 2 sequence containing from 5′ to 3′ a second partial Read 2 sequence and the first partial Read 2 sequence. The method results in the preparation of mitochondrial cDNA sequencing library. The first forward primer, the first reverse primer, the second reverse primer, and the second forward primer do not form primer dimers that prevent the production of amplicons during a PCR reaction containing two or more of the first forward primer, first reverse primer, second reverse primer, and second forward primer.

In any aspect of the disclosure, or embodiments thereof, in step c) the second forward primer contains in order from 5′ to 3′ end the nucleotide sequence AATGATACGGCGACCACCGAGATCTACAC (SEQ ID NO: 4), a first indexing sequence, and the nucleotide sequence ACACTCTTTCC (SEQ ID NO: 5), and the second reverse primer contains in order from 5′ to 3′ the nucleotide sequence CAAGCAGAAGACGGCATACGAGAT (SEQ ID NO: 6), a second indexing sequence, and the nucleotide sequence GTGACTGGAGTTCCTTGGCACCCGAGAATTCCA (SEQ ID NO: 7).

In any aspect of the disclosure, or embodiments thereof, the second forward primer and second reverse primer each contain a nucleotide sequence selected from those listed in Table 3.

In any aspect of the disclosure, or embodiments thereof, in step b) the first reverse primer contains a targeting sequence listed in Table 2, or a variant thereof with up to 5 total nucleotide alterations, where the targeting sequence is complementary to a mitochondrial cDNA sequence present in the cDNA library.

In any aspect of the disclosure, or embodiments thereof, step a) further involves two or more first PCR reactions, where one of the first PCR reactions involves using a first unique set of first reverse primers containing nucleotide sequences corresponding to a first RNA sequencing primer mixture of Table 2. Another of the first PCR reactions involves using a second unique set of first reverse primers containing nucleotide sequences corresponding to a second RNA sequencing primer mixture of Table 2.

In any aspect of the disclosure, or embodiments thereof, step a) involves twelve first PCR reactions, where each of the twelve first PCR reactions involves using a unique set of first reverse primers, each unique set of reverse primers containing nucleotide sequences corresponding to an RNA sequencing primer mixture of Table 2.

In any aspect of the disclosure, or embodiments thereof, the method further involves pooling the first PCR amplicons from each of the twelve first PCR reactions at the following volumetric ratios: 40 volumetric units of each of the first PCR amplicons corresponding to primer mixes 1-9, to 12 volumetric units of the first PCR amplicon mixture corresponding to primer mix 10, to 8 volumetric units of the first PCR amplicon mixture corresponding to each of primer mixes 11 and 12.

In any aspect of the disclosure, or embodiments thereof, in step b) the molar ratio of the first forward primer to the first reverse primer (F:R) is from about 1:2 to about 1:3. In any aspect of the disclosure, or embodiments thereof, in step c) the molar ratio of the second forward primer to the second reverse primer is from about 1:4 to about 1:6. In any aspect of the disclosure, or embodiments thereof, the amount of the cDNA library used in each or the first PCR reaction is from about 1 ng to about 25 ng. In any aspect of the disclosure, or embodiments thereof, the total mass of the cDNA library used in each or the first PCR reaction is from about 1 ng to about 5 ng.

In any aspect of the disclosure, or embodiments thereof, an annealing temperature used in each first PCR reactions is about 60° C., and each first PCR reactions involves at least about 10 extension cycles. In any aspect of the disclosure, or embodiments thereof, each first PCR reactions involves about 13 extension cycles. In any aspect of the disclosure, or embodiments thereof, the annealing temperature used in the second PCR reaction is about 54° C.

In any aspect of the disclosure, or embodiments thereof, the method further involves pooling the first PCR amplicons obtained in the first PCR reaction(s) and cleaning the first PCR amplicons using solid-phase reverse immobilization (SPRI) beads. In any aspect of the disclosure, or embodiments thereof, cleaning the first PCR amplicons involves eluting the first PCR amplicons from the SPRI beads using an elution volume of a nuclease-free solution containing water, where the elution volume is from about 50 μL to about 300 μL. In any aspect of the disclosure, or embodiments thereof, the elution volume is about 100 μL.

In any aspect of the disclosure, or embodiments thereof, at least 40% of the sequencing library is sequenceable, and where sequenceability is measured using a KAPA Library Quantification kit.

In any aspect of the disclosure, or embodiments thereof, the polynucleotide of step a) further contains a barcode sequence identifying the cell from which the polynucleotide was derived. In any aspect of the disclosure, or embodiments thereof, the polynucleotides of step a) each further contains a Unique Molecular Identifier (UMI) sequence that identifies an mRNA molecule. In any aspect of the disclosure, or embodiments thereof, the Unique Molecular Identifier (UMI) sequence identifies PCR duplicates present in the mitochondrial cDNA sequencing library. In any aspect of the disclosure, or embodiments thereof, the polynucleotide of step a) further contains a template switch oligomer containing the nucleotide sequence TTTCTTATATGGG (SEQ ID NO: 8).

In any aspect of the disclosure, or embodiments thereof, the cell of step a) is an isolated cell present in an aqueous solution-in-oil emulsion, where each aqueous solution droplet within the emulsion contains a single cell, a gel bead, and reagents appropriate for the preparation of a cDNA library within each aqueous solution droplet.

In any aspect of the disclosure, or embodiments thereof, the cDNA library contains polynucleotides derived from single cells present in a biological sample.

In any aspect of the disclosure, or embodiments thereof, the biological sample is a blood sample, a bone marrow sample, or a biopsy. In any aspect of the disclosure, or embodiments thereof, the biological sample is from a subject having a neoplasia, a preneoplasia, an autoimmune disease, a cardiovascular disease, or a clone of cells. In any aspect of the disclosure, or embodiments thereof, the neoplasia is a multiple myeloma. In any aspect of the disclosure, or embodiments thereof, the subject is a mammal. In any aspect of the disclosure, or embodiments thereof, the subject is a human. In any aspect of the disclosure, or embodiments thereof, the cell is an isolated cell.

In any aspect of the disclosure, or embodiments thereof, the sequencing library is sequenced using next generation sequencing. In any aspect of the disclosure, or embodiments thereof, the next generation sequencing involves sequencing-by-synthesis, where the sequence read lengths are from about 50 to about 500 bases in length. In any aspect of the disclosure, or embodiments thereof, the sequence coverage for mitochondrial cDNA in the library is at least about 50×. In any aspect of the disclosure, or embodiments thereof, the sequence coverage is at least about 80×. In any aspect of the disclosure, or embodiments thereof, at least 90% of the sequence reads map to the mitochondrial transcriptome. In any aspect of the disclosure, or embodiments thereof, the method detects alterations in the sequence of a mitochondrial gene relative to a wild-type mitochondrial gene. In any aspect of the disclosure, or embodiments thereof, the mitochondrial gene is ATP8, ND4L, or ND6.

In any aspect of the disclosure, or embodiments thereof, the clone cells are neoplastic cells. In any aspect of the disclosure, or embodiments thereof, the biological sample is a bone marrow sample or a blood sample. In any aspect of the disclosure, or embodiments thereof, the neoplastic cells are from a hematological malignancy.

In any aspect of the disclosure, or embodiments thereof, the method further involves using the identified SNVs and/or CNVs in the sequence data to cluster the sequence data and identify clonal and/or subclonal cell populations in the biological sample.

In any aspect of the disclosure, or embodiments thereof, the two time points are separated by 1 wk, 2 wks, 4 wks, 6 months, a year, or longer. In any aspect of the disclosure, or embodiments thereof, a first time point is a time prior to administration of the treatment.

In any aspect of the disclosure, or embodiments thereof, the set of compositions contains twelve compositions.

In any aspect of the disclosure, or embodiments thereof, the primer of c) contains a nucleotide sequence selected from those RNA sequencing primers sequences listed in Table 2

In any aspect of the disclosure, or embodiments thereof, the first partial Read 1 sequence is from about 15 to about 25 nucleotides in length. In any aspect of the disclosure, or embodiments thereof, the second partial Read 1 sequence is from about 5 to about 20 nucleotides in length. In any aspect of the disclosure, or embodiments thereof, the first partial Read 2 sequence is from about 10 to about 20 nucleotides in length. In any aspect of the disclosure, or embodiments thereof, the second partial Read 2 sequence is from about 15 to about 25 nucleotides in length. In any aspect of the disclosure, or embodiments thereof, the P7 sequence is from about 20 to about 40 nucleotides in length. In any aspect of the disclosure, or embodiments thereof, the P5 sequence is from about 20 to about 40 nucleotides in length. In any aspect of the disclosure, or embodiments thereof, the polynucleotides of step a) each further contain a Unique Molecular Identifier (UMI) sequence that identifies an mRNA molecule and a barcode sequence identifying the cell from which the polynucleotide was derived. In any aspect of the disclosure, or embodiments thereof, the second forward primer contains from 5′ to 3′ a P5 sequence, a first indexing sequence, and the second partial Read 1 sequence, and where the second reverse primer contains in order from 5′ end to 3′ end a P7 sequence, a second indexing sequence, and a Read 2 sequence containing from 5′ to 3′ the second partial Read 2 sequence and the first partial Read 2 sequence.

Compositions and articles defined herein were isolated or otherwise manufactured in connection with the examples provided below. Other features and advantages of the embodiments of the disclosure will be apparent from the detailed description, and from the claims.

### Definitions

Unless defined otherwise, all technical and scientific terms used herein have the meaning commonly understood by a person skilled in the art to which the aspects and embodiments of the disclosure belong. The following references provide one of skill with a general definition of many of the terms used in this disclosure: Singleton et al., Dictionary of Microbiology and Molecular Biology (2^{nd} ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease. In embodiments, the disease is a neoplasia, cancer, or solid tumor.

By “alteration” is meant a change in the structure, expression levels, or activity of a polynucleotide or polypeptide as detected by standard art known methods, such as those described herein. The alteration can be an increase or a decrease. As used herein, an alteration includes a 10% change in expression levels, a 25% change, a 40% change, and a 50% or greater change in expression levels.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “clone” or “clone of cells” is a group of cells that share a common ancestry, meaning they are derived from the same cell. In certain embodiments, new mutations arise over time in a clonal population giving rise to sub-clonal populations of cells. As used herein, the term “clonal structure” refers to the assessment of clonal contributions of various clones and sub-clones in a heterologous population (e.g., in a neoplasia, cancer, tumor). In certain embodiments, the clonal structure is determined before and/or after a treatment or is used to monitor disease progression in the presence or absence of therapy. In certain embodiments, a clone of a cell may not be malignant.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “cDNA polynucleotide” or “cDNA” is meant a DNA molecule prepared through reverse transcription from an mRNA molecule. In some embodiments, the cDNA is derived from one or more mitochondria of a cell. If the cDNA is derived from one or more mitochondria of a cell, it may be referred to as “mitochondrial cDNA.”

By “full-length cDNA polynucleotide” or “full-length cDNA” is meant a cDNA polynucleotide comprising a nucleotide sequence encoding a full-length open reading frame of an mRNA molecule. In some embodiments, the open reading frame encodes a polypeptide and includes a start codon and ends at a stop codon. In some embodiments, a cDNA polynucleotide comprises a nucleotide sequence comprising nucleotides corresponding to a full-length open reading frame corresponding to a wild-type version of an mRNA molecule.

By “detect” is meant identifying the presence, absence, or amount of an analyte to be detected. In embodiments, the analyte is a polypeptide, polynucleotide, or fragment thereof.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “coverage” for a nucleobase in the context of nucleotide sequencing is meant the number of sequencing reads containing said nucleobase.

By “decrease” is meant a negative alteration. In embodiments, the decrease is by about 1%, 5%, 10%, 25%, 30%, 50%, 75%, 100%, or more, or by 1.5-fold, 2-fold, 3-fold, 4-fold, 5-fold, 6-fold, 7-fold, 8-fold, 9-fold, 10-fold, 25-fold, 50-fold, 75-fold, 100-fold, or more

By “disease” is meant any condition or disorder that damages or interferes with the normal function of a cell, tissue, or organ. Examples of diseases include neoplasias, such as tumors or cancers. In some embodiments, the neoplasia is a multiple myeloma. In some embodiments the neoplasia is a hematological malignancy or a premalignancy. In some embodiments, the disease is Clonal Hematopoiesis of Indeterminate Potential (CHIP), Monoclonal Gammopathy of Indeterminate Potential (MGIP), Monoclonal B cell Lymphocytosis (MBL), or Monoclonal Gammopathy of Undetermined Significance (MGUS). The disease may be an autoimmune disease. The disease may be a disease associated with inflammation. The disease may be a pulmonary disease or dementia. The disease may be a disease associated with a clonal expansion of cells in a subject.

By “effective amount” is meant the amount of an agent required to ameliorate the symptoms of a disease relative to an untreated patient. The effective amount of active compound(s) used to practice the embodiments of the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. In embodiments, the portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, 1000, 1,100, 1,200, 1,300, 1,400, 1,500, 1,600, 1,700, 1,800, 1,900, or 2,000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increase” is meant to alter positively relative to a reference. An increase may be by 1%, 5%, 10%, 25%, 30%, 50%, 75%, 100%, or more, or by 1.5-fold, 2-fold, 3-fold, 4-fold, 5-fold, 6-fold, 7-fold, 8-fold, 9-fold, 10-fold, 25-fold, 50-fold, 75-fold, 100-fold, or more.

By “indexing sequence” is meant a nucleotide sequence that provides for the identification of a sequencing library comprising the indexing sequence. Adding indexing sequences to sequence libraries allows the libraries to be pooled and sequenced together. In some embodiments, a sequencing library contains two unique indexing sequences (i.e., is dual indexed).

The terms “isolated,” “purified,” or “biologically pure” refer to material that is free to varying degrees from components which normally accompany it as found in its native state.

“Isolate” denotes a degree of separation from an original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide as disclosed herein is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule described herein is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, and or at least 99%, by weight of a polypeptide as disclosed herein. An isolated polypeptide as disclosed herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein, polynucleotide, clinical indicator, or other analyte having an alteration in expression level or activity that is associated with a developmental state, condition, disease, or disorder.

As used herein, “obtaining” as in “obtaining an agent” includes synthesizing, purchasing, or otherwise acquiring the agent.

By “polynucleotide” or “nucleic acid molecule” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. In embodiments, such modified or substituted oligonucleotides are used over native forms because of properties such as, for example, enhanced stability in the presence of nucleases.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects, the disclosure embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made.

Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

“Primer set” means a set of oligonucleotides that may be used, for example, for PCR. A primer may comprise about or at least about 2, 4, 6, 8, 10, 12, 14, 16, 18, 20, 30, 40, 50, 60, 80, 100, 200, 250, 300, 400, 500, 600, or more primers.

By “reference” is meant a standard or control condition. In various embodiments, a reference is a healthy subject. In some cases, a reference is a method lacking one or more elements and/or having one or more altered elements relative to a method of interest.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween. In some embodiments, the sequence of a cDNA derived from a single neoplastic cell is compared to the sequence of a corresponding wild-type DNA encoding the same protein as the cDNA (i.e., a reference sequence) and is compared to the sequence of a cDNA derived from another neoplastic cell within the tumor encoding the same protein as the cDNA. A cDNA derived from a first neoplastic cell may be compared to a cDNA derived from a cDNA derived from a second neoplastic cell that belongs to the same clone or that belongs to a different clone than the first neoplastic cell. A reference sequence may be a mitochondrial genome from a healthy subject. A representative mitochondrial genome sequence is provided at NCBI Reference Sequence Accession No. NC_012920.1.

By “sequencing by synthesis” is meant a DNA sequencing method involving detection of light emitted by fluorescently-labelled bases as they are incorporated into a growing DNA strand. A non-limiting example of a sequencing by synthesis-based next-generation sequencing method is Illumina sequencing. In some embodiments, a sequencing by synthesis sequencing method is a short-read sequencing method where the sequence reads are less than about 50 bases, 100 bases, 150 bases, 200 bases, 250 bases, 300 bases, 350 bases, 400 bases, 450 bases, or 500 bases in length. In some embodiments, the sequence reads in a short-read sequencing method are about or at least about 50 bases, 100 bases, 150 bases, 200 bases, 250 bases, 300 bases, 350 bases, 400 bases, or 450 bases in length.

By “substantially identical” is meant a polypeptide or nucleic acid molecule exhibiting at least 50% identity to a reference amino acid sequence (for example, any one of the amino acid sequences described herein) or a nucleic acid sequence (for example, any one of the nucleic acid sequences described herein). In embodiments, such a sequence is at least 60%, at least 80% or 85%, or at least about 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT,

GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “subject” is meant an animal. The animal can be a mammal. The mammal can be a human or non-human mammal, such as a bovine, equine, canine, ovine, rodent, or feline. Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15,16, 17, 18, 19, 20, 21, 22, 23, 24, 25, 26, 27, 28, 29, 30, 31, 32, 33, 34, 35, 36, 37, 38, 39, 40, 41,42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “unique molecular identifier (UMI)” is meant a nucleotide sequence that provides for the identification of cDNA sequences derived from a common mRNA molecule. Inclusion of UMIs in cDNA libraries used in the preparation of sequencing libraries allows for down-stream corrections for PCR biases or other similar methodology-based sequence duplications that may have been presented during preparation of the cDNA libraries and/or the sequencing libraries.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art. In some cases, a range of normal tolerance in the art is within 1 or 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides boxplots showing normalized gene expression for mitochondrial genes determined through the analysis of whole-transcriptome sequencing data prepared using either 5′-end single-cell RNA sequencing (top panel) or 3′-end single-cell RNA sequencing, where the cDNA libraries sequenced were prepared using kits available from 10X Genomics (bottom panel).

FIG. 2 provides plots showing BioAnalyzer traces of cDNA prepared using a Chromium-5′ Single Cell Gene Expression kit available from 10X Genomics following targeted amplification with primers specific for mitochondrial cDNA. The cDNA was prepared as described in FIG. 10C using the RNA sequencing primers (RSPs) listed in Table 2. In FIG. 2, the terms “RSP_MT-CO3_4”, “RSP_MT-CYB_6,” “RSP_MT-ND5_9,” “RSP_MT-ND3_2,” “RSP_MT-CO1_7,” “RSP_MT-ND4_7,” and “RSP_MT-ND4L_2” indicate the names of the RNA sequencing primers corresponding to the indicated plot, the term “XV-P5-i5-BC01” indicates a plot corresponding to cDNA amplified using only the forward primer in the 1^{st} PCR depicted in FIG. 10C, the term “(−)CTRL” indicates a plot corresponding to a negative control, and MM1S_cDNA indicates a plot corresponding to unamplified cDNA from multiple myeloma cell line MM.1S.

FIG. 3 provides plots showing BioAnalyzer traces of cDNA prepared using a Chromium-5′ Single Cell Gene Expression kit available from 10X Genomics following targeted amplification with primers specific for mitochondrial cDNA. The libraries were prepared as described in FIGS. 11C using the Read 1 primer listed in Table 1 and RNA sequencing primers (RSPs) listed in Table 2. In FIG. 3, the terms above each plot (e.g., NDS_1) refer to the RSP corresponding to the indicated plot, where the prefix “RSP_MT-” from Table 2 has been removed from the terms listed above each plot as compared to Table 2.

FIG. 4 provides bar graphs showing the log2 mean depth of sequencing coverage for nucleotides amplified using the RNA sequencing primers (RSPs) of Table 2, where the primers corresponding to each “Primer Mix” are indicated in Table 2. The left panel of FIG. 4 relates to sequencing data obtained according to the methods of the disclosure where, following the 1st PCR of the method depicted in FIGS. 11A-11E, the resulting amplicon mixtures obtained using the different primer mixes were pooled as follows: 40 μL of each of the amplicon mixtures corresponding to primer mixes 1-9, 35 μL of the amplicon mixture corresponding to primer mix 10, and 32 μL of each of the amplicon mixtures corresponding to primer mixes 11 and 12. The right panel of FIG. 4 relates to sequencing data obtained according to the methods of the disclosure where, following the 1^{st} PCR of the method depicted in FIGS. 11A-11E, the resulting amplicon mixtures obtained using the different primer mixes were pooled as follows: 40 μL of each of the amplicon mixtures corresponding to primer mixes 1-9, 12 μL of the amplicon mixture corresponding to primer mix 10, and 8 μL of each of the amplicon mixtures corresponding to primer mixes 11 and 12.

FIG. 5 provides a plot showing the impact of different elution volumes used in a solid-phase reversible immobilization (SPRI) bead clean-up of the pooled amplicon mixtures prepared according to the method described above for FIG. 4 on the proportion (% yield) of total library DNA prepared according to the method depicted in FIGS. 11A-11E that was sequenceable. The total library DNA was measured using the stain PicoGreen™, and the amount of library DNA predicted to be sequenceable was determined using a KAPA Library Quantification Kit (see y-axis of FIG. 5). The following elution volumes (i.e., μL nuclease free water used to elute the pooled amplicon mixtures from the SPRI beads) were evaluated: 50 μL, 100 μL, 150 μL, 200 μL, 300 μL, and 400 μL. 12 μL from each elution were used for the 2^{nd} PCR reaction. In FIG. 5, the Greek letter “μ” is represented as “μ”. The impact of the concentration of the primers (i.e., PrimerMix volume in μL added to the PCR mixture) used in the 2^{nd} PCR, as depicted in FIG. 11D, was also evaluated (see x-axis of FIG. 5).

FIG. 6 provides bar plots showing that libraries prepared using elution volumes of 300 μL or 100 μL, as described for FIG. 5, had a similar distribution of sequencing coverage for each cell; however, for the elution volume of 300 μL, 84% of total reads (i.e., of 248 million total reads (n=248M)) mapped to mitochondrial DNA (chrM), and for the elution volume of 100 μL, 95% of total reads (i.e., of 277 million total reads (n=277M)) mapped to chrM.

FIG. 7 provides a plot showing that altering the conditions for the 2^{nd} PCR increased the proportion (% yield) of total library DNA prepared according to the method depicted in FIGS. 11A-11E that was sequenceable. In particular, FIG. 7 provides a plot showing % yield associated with using the “10× condition” or “Regular condition,” as described below, with elution volumes of 100 μL or 300 μL, as described in FIG. 5, and at different concentrations of the forward and reverse primers. The x-axis of FIG. 7 indicates the relative total concentration of the forward and reverse primers (PrimerMix) used in the 2^{nd} PCR reaction, where 1× indicates a total concentration of 0.75 μM for the reverse primer and 0.15 μM for the forward primer (i.e., a forward primer (F) to reverse primer (R) ratio of 5:1) for the “10× condition,” 2× indicates a concentration of 1.5 μM for the reverse primer and 0.3 μM for the forward primer for the “10× condition,” etc. and 1× indicates a concentration of 0.75 μM for the reverse primer and 0.15 μM for the forward primer for the “Regular condition,” 2× indicates a total concentration of 1.5 μM for the reverse primer and 0.3 μM for the forward primer for the “Regular condition,” etc. In FIG. 7 “Regular condition” indicates the following conditions for the 2^{nd} PCR reaction: an initial denaturation time of 45 sec and an annealing temperature of 60° C. In FIG. 7, “10X condition” indicates the following conditions for the 2^{nd} PCR reaction: an initial denaturation time of 45 sec, and an annealing temperature of 54° C. The total library DNA was measured using the stain PicoGreen™, and the amount of library DNA predicted to be sequenceable was determined using a KAPA Library Quantification Kit (see y-axis of FIG. 7).

FIGS. 8A and 8B provide bar plots showing mean transcriptome sequence coverage relative to position on the mitochondrial genome (chrM) achieved using the long-read sequencing protocol of Penter L, et al. Nat Commun 15:32 (2024) (FIG. 8A) as compared to the 5′-end single-cell mitochondrial RNA sequencing method of the present disclosure (FIG. 8B). The y-axis was cut-off at 200 for visualization. The genomic locations corresponding to sequenced transcripts are indicated above the bars of FIG. 8A.

FIGS. 9A and 9B provide a heatmap and a uniform manifold and projection (UMAP) plot showing single-cell lineage tracing for tumor cells from a patient with smoldering multiple myeloma (SMM) carried out using the 5′-end single-cell mitochondrial RNA sequencing method of the present disclosure. FIG. 9A provides a heatmap of mitochondrial single nucleotide variants (SNVs) in the tumor cells. The cells and variants were ordered by agglomerative hierarchical clustering. The bars at the top of FIG. 9A visualize clone demarcations based on mitochondrial SNVs (top bar, 12 total clones identified) or copy number variants (CNVs) (bottom bar, 3 clones identified) identified using Numbat. Numbat is a haplotype-enhanced CNV caller for single-cell RNA sequencing (scRNA-seq) data. FIG. 9B provides a UMAP plot showing embedding of tumor cells, where the different clones are identified by different shades of grey. The clones (i.e., cell lineages) were identified using either hierarchical clustering of mitochondrial SNVs (“HC”) or CNV clustering by Numbat (“Numbat”).

FIGS. 10A-10E present schematic diagrams corresponding in order to steps of an embodiment of a method for 5′-end single-cell sequencing of mitochondrial RNA using a Gel Beads-in-emulsion (GEM) method. FIG. 10A provides a schematic diagram presenting steps of a 10X method for preparation of cDNA from an mRNA sample. FIG. 10B provides a schematic diagram showing full-length cDNA prepared using the product of the Gel Beads-in-emulsion method of FIG. 10A through the use of PCR primers complementary to Read 1 and the Non Poly-dT sequence. FIG. 10A discloses “aaaaaaaaaaaaaaaaaaaa” as SEQ ID NO: 196. FIG. 10C presents a schematic diagram showing a 1^{st} PCR reaction where the sequences containing the sequences containing the cDNA insert correspond to the amplicons of FIG. 10B, the primer to the left containing the regions P5, i5, and Read 1 is a forward primer, and the primer to the right containing the regions mtcDNA and Partial Read 2 is a reverse primer. FIG. 10D presents a schematic diagram showing a 2^{nd} PCR reaction, where the template for the PCR reaction is the amplicon resulting from the 1^{st} PCR reaction and contains the regions i5, N16, N10, and mtcDNA. In FIG. 10D, the primer to the left containing a P5 region is a forward primer, and the primer to the right containing the regions Read 2, i7, and P7 is a reverse primer. FIGS. 10C-10D disclose SEQ ID NOS 197-198, 3, 199-207, 4, and 208-210, respectively, in order of appearance.

FIG. 10E provides a schematic diagram showing an amplicon resulting from the 2^{nd} PCR. In FIGS. 10A-10E, the term “TSO” indicates a template switch oligo capable of hybridizing to untemplated C nucleotides produced by a reverse transcriptase during reverse transcription, the term “UMI” indicates a unique molecular identifier, the term “10x barcode” indicates a barcode sequence, the term “Read 1” indicates an oligonucleotide sequence, the grey circle indicates a gel bead, the term “GEM” indicates a Gel Beads-in-emulsions method for production of cDNA carried out according to a protocol provided together with kits and apparatus (e.g., Chromium Next GEM Chip K) commercially available from 10X Genomics, the term “i5” indicates an index, the term “i7” indicates an index, the term “mtcDNA” indicates a mitochondrial cDNA sequence, the term “N16” indicates a barcode, the term “N10” indicates a unique molecular identifier, and the term “mtcDNA” indicates a targeting sequence, such as those listed in Table 2. FIGS. 10A, 10B, and 10E are taken from the user guide (Document No. CG000331 Rev E) available from 10X Genomics for the Chromium Next GEM Single Cell 5′ Reagent Kits v2 (Dual Index) kits.

FIGS. 11A-11E present schematic diagrams corresponding in order to steps of a method developed as described in Example 1 for 5′-end single-cell sequencing of mitochondrial RNA using a Gel Beads-in-emulsion (GEM) method. FIG. 11A provides a schematic diagram presenting steps of a 10X method for preparation of cDNA from an mRNA sample. FIG. 11A discloses “aaaaaaaaaaaaaaaaaaaa” as SEQ ID NO: 196. FIG. 11B provides a schematic diagram showing an amplicon prepared from the product of the Gel Beads-in-emulsion method of FIG. 11A through the use of PCR primers complementary to Read 1 and the Non Poly-dT sequence. FIG. 11C presents a schematic diagram showing a 1^{st} PCR reaction where the sequences containing the sequences containing the cDNA insert correspond to the amplicons of FIG. 11B, the primer to the left containing the region Read 1 is a forward primer, and the primer to the right containing the regions mtcDNA and Partial Read 2 is a reverse primer. FIG. 11D presents a schematic diagram showing a 2^{nd} PCR reaction, where the template for the PCR reaction is the amplicon resulting from the 1^{st} PCR reaction and contains the regions N16, N10, and mtcDNA. In FIG. 11D, the primer to the left containing the regions P5 and i5 is a forward primer, and the primer to the right containing the regions Read 2, i7, and P7 is a reverse primer. FIGS. 11C-11D disclose SEQ ID NOS 197-198, 3, 2, 201-202, 211-212, 206-207, and 213-216, respectively, in order of appearance. FIG. 11E provides a schematic diagram showing an amplicon resulting from the 2^{nd} PCR. In FIGS. 11A-11E, the term “TSO” indicates a template switch oligo capable of hybridizing to untemplated C nucleotides produced by a reverse transcriptase during reverse transcription, the term “UMI” indicates a unique molecular identifier, the term “10x barcode” indicates a barcode sequence, the term “Read 1” indicates an oligonucleotide sequence, the grey circle indicates a gel bead, the term “GEM” indicates a Gel Beads-in-emulsions method for production of cDNA carried out according to a protocol provided together with kits and apparatus (e.g., Chromium Next GEM Chip K) commercially available from 10X Genomics, the term “i5” indicates an index, the term “i7” indicates an index, the term “mtcDNA” indicates a mitochondrial cDNA sequence, the term “N16” indicates a barcode, the term “N10” indicates a unique molecular identifier, and the term “mtcDNA” indicates a targeting sequence, such as those listed in Table 2. FIGS. 11A, 11B, and 11E are taken from the user guide (Document No. CG000331 Rev E) available from 10X Genomics for the Chromium Next GEM Single Cell 5′ Reagent Kits v2 (Dual Index) kits.

FIG. 12 provides a schematic diagram showing an experimental setup for carrying out a method for 5′-end single-cell sequencing of mitochondrial RNA of the disclosure. The RNA sequencing primer mixes (e.g., PCR1 Primer Mixes 1, 2, 3, 4, 5, 6, 7, 8, 9, 10, 11, and 12) are prepared as described in Table 2 on a 96-well mix plate and then combined with different barcoded full-length cDNA samples (e.g., Full-length cDNA libraries S1, S2, S3, and S4) to yield a PCR1 plate prepared for carrying out the 1^{st} PCR reaction (PCR1), as depicted in FIG. 11C, where each row of the plate corresponded to a different sample and each column on the plate corresponded to a different RNA sequencing primer mix. The RNA sequencing primer mixes contained forward and reverse primers at a ratio of 1:5, where the forward primer was the primer listed in Table 1 and the reverse primers were the sample indexing PCR primers of Table 2. Following the 1^{st} PCR, the reaction products for each of the 12 corresponding amplification reactions were pooled for each barcoded full-length cDNA sample (e.g., S1, S2, S3, and S4) and cleaned. These pooled and cleaned reaction products were then used as templates for the 2^{nd} PCR (PCR2) to yield reaction mixes (e.g., S1 BC01, S2 BC02, S3 BC03, and S4 BC04, where the terms BC01, etc. indicate a unique set of sample indexing primers used for each PCR2 reaction), where the 2^{nd} PCR was a sample-indexing PCR adding a different pair of indices to the pooled amplicons corresponding to each sample. The 2^{nd} PCR yields cDNA sequencing libraries that are then sequenced using next generation sequencing (NGS).

FIG. 13 provides a schematic diagram showing how i5, Read 2, Read 1, and i7 primers were used to sequence the amplicons of FIGS. 10E or 11E using Illumina sequencing carried out using a NovaSeq™M 6000 v1.5 Reagent Kit. FIG. 13 discloses SEQ ID NOS 217-221, 7, 2, and 222-226, respectively, in order of appearance.

FIGS. 14A and 14B provide box-and-whisker plots showing that all of the primers of Table 2 performed well in the method described in FIGS. 11A-11E and 12, as indicated by the depth of sequencing coverage achieved for amplicons corresponding to each primer. The elution volume used to prepare the libraries that were sequenced to prepare the data presented in FIGS. 14A and 14B was 100 μL and the 2^{nd} PCR primer concentrations were 0.75 μM for the reverse primer and 0.15 μM for the forward primer. The boxes of FIG. 14A are ordered by primer mix and the boxes of FIG. 14B are ordered by gene.

FIG. 15 provides bar plots showing that libraries prepared using an annealing temperature of 60° C. or 54° C. in the 2^{nd} PCR reaction described in FIGS. 11A-11E were associated with similar numbers of reads mapping to mitochondrial DNA (chrM), with 95.4% of reads (n=249 M) mapping to chrM when the annealing temperature of 60° C. was used (left panel of FIGS. 15) and 95% of reads (n=293 M) mapping to chrM when the annealing temperature of 54° C. was used (right panel of FIG. 15).

FIG. 16 provides bar plots showing that libraries prepared using different strategies (v1, v2, and v3, as indicated above each plot of FIG. 16) for pooling the RNA sequencing primer (RSP) mixes of Table 2 for use in the 1^{st} PCR reaction described in FIGS. 11A-11E were associated with small changes in the numbers of reads mapping to mitochondrial DNA (chrM) and minimal changes in depth of sequencing. In FIG. 16, the terms “v1,” “v2,” or “v3” represent the pooling strategy used to prepare the data represented in the indicated bar plot. When pooling strategy v1 was used, 96.2% of reads (n=345 M) mapped to chrM, when pooling strategy v2 was used, 95.4% of reads (n=350 M) mapped to chrM, and when pooling strategy v3 was used, 94.3% of reads (n=300M) mapped to chrM. Pooling strategy v1 involved pooling the amplicon mixtures by using 40 μL of each of the amplicon mixtures corresponding to primer mixes 1-9, 35 μL of the amplicon mixture corresponding to primer mix 10, and 32 μL of each of the amplicon mixtures corresponding to primer mixes 11 and 12. Pooling strategy v2 involved pooling the amplicon mixtures by using 40 μL of each of the amplicon mixtures corresponding to primer mixes 1-9, 32 μL of the amplicon mixture corresponding to primer mix 10, and 20 μL of each of the amplicon mixtures corresponding to primer mixes 11 and 12. Pooling strategy v3 involved pooling the amplicon mixtures by using 40 μL of each of the amplicon mixtures corresponding to primer mixes 1-9, 12 μL of the amplicon mixture corresponding to primer mix 10, and 8 μL of each of the amplicon mixtures corresponding to primer mixes 11 and 12.

FIG. 17 provides bar graphs showing the log2 mean depth of sequencing coverage for nucleotides amplified using the RNA sequencing primers (RSPs) of Table 2, where the primers corresponding to each “Primer Mix” are indicated in Table 2, and where the libraries sequenced were prepared as described for FIG. 16. In FIG. 17, the terms “v1,” “v2,” or “v3” represent the pooling strategy used to prepare the data represented in the indicated bar graph. The pooling strategies are described above for FIG. 16.

FIGS. 18A and 18B provide sets of plots and bar plots showing that libraries prepared using pooling strategy v3 described for FIG. 16 had an approximately 2-fold decrease in sequence coverage of amplicons corresponding to RNA sequencing primer (RSP) mixes 11 and 12 (i.e., within the RNR1 and RNR2 genes because primer mixes 11 and 12 contained primers amplifying portions of the genes RNR1 or RNR2). The terms above each plot or bar graph of FIGS. 18A and 18B following the prefix “MT-” indicate the mitochondrial (i.e., “MT”) gene corresponding to teach plot or bar graph.

### DETAILED DESCRIPTION

The present disclosure features compositions and methods that are useful for 5′-end single-cell sequencing of mitochondrial RNA.

The aspects of the disclosure are based, at least in part, upon the discovery of a method for effectively sequencing mitochondrial mRNA using 5′ single-cell mRNA sequencing. The methods herein are founded on a droplet-based scRNA-seq technology (e.g., nanoliter-scale Gel Beads-in-emulsion (GEMs)), where mRNA is separated and reverse transcribed to cDNA for each cell. To develop the method, about 70 primers targeting the entirety of the mitochondrial transcriptome were designed and tested to demonstrate specificity of mitochondrial mRNA amplification. The cDNA generated from the mRNA was barcoded and sequenced using a 10X Genomics 5′ assay. Primers and PCR conditions were evaluated and designed for use in the 5′ single-cell mRNA sequencing. Specifically, the length of the primers for a first round of PCR and the ratio of the forward to reverse primers (F:R) used in the 5′ single-cell mRNA sequencing was designed to enable specific amplification of targeted amplicons. The amount of input cDNA used in the method for each first PCR reaction was between about 20 ng to about 10 ng.

However, data provided herein indicates that as little as 0.5 ng to 2.5 ng cDNA may be used for each first PCR reaction. In embodiments, less than about 2.5 ng of cDNA is used for each first PCR reaction. Additionally, the methods of the disclosure were designed taking into account the forward and reverse primers and the ratio thereof (F:R) used in a second round of PCR. The methods of the disclosure also took into account the input amount of cDNA for the second round of PCR and thermal cycler conditions for the second round of PCR. Also, the methods of the disclosure account for the amount of amplicons pooled for use as templates in the second round of PCR. A laboratory developed test (LDT) utilizing the 5′ single-cell mRNA sequencing method provided herein may involve the coupling of single-cell RNA-seq profiling with single-cell mitochondrial transcript sequencing to detect malignant cells and subclones within patient samples, which may be useful for diagnostics and prognostication. Also, kits for use in the methods of the present disclosure may be suitable for use with single-cell RNA sequencing kits (e.g., to enable research-level endeavors). In some embodiments, the 5′ single-cell mRNA sequencing methods of the disclosure may be combined with single-cell RNA sequencing, single-cell B cell receptor/T cell receptor (BCR/TCR) sequencing, and/or single-cell sequencing of targeted amplicons for somatic mutation detection.

### Mitochondrial Genomes

Mitochondria are dynamic organelles that are present in almost all eukaryotic cells and play a crucial role in multiple cellular pathways. The human mitochondrial genome is a double-stranded, circular molecule of 16,569 bp that contains 37 genes coding for two rRNAs, 22 tRNAs and 13 polypeptides (NCBI Reference Sequence Accession No. NC_012920.1). These mRNAs are transcribed and then translated within the mitochondrial matrix by a dedicated, unique, and highly specialized machinery. Mitochondrial mRNAs are polyadenylated by a mitochondrial poly (A) polymerase during or immediately after cleavage, whereas the 3′-ends of the two rRNAs are post-transcriptionally modified by the addition of only short adenyl stretches. Somatic mutations in the mitochondrial genome (mtDNA) provide a compelling alternative for determining lineages and clonal structure for cancers or tumors because multiple studies have shown that each human cell contains hundreds-to-thousands of mitochondrial genomes with diverse and often manifold mutations at detectable levels of heteroplasmy.

### Library Preparation for Sequencing

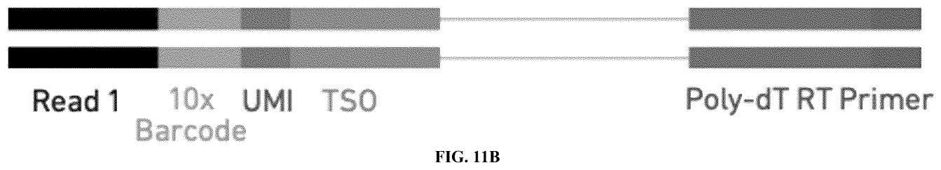

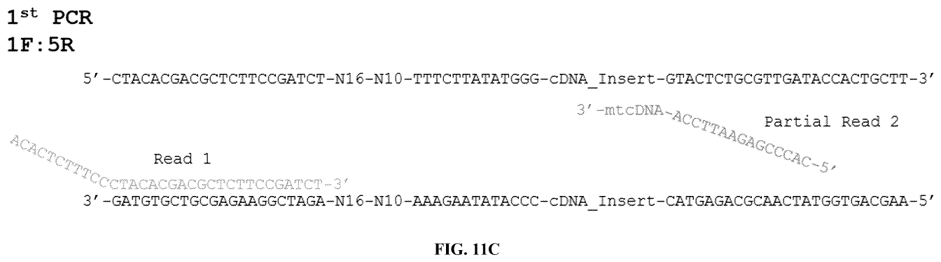

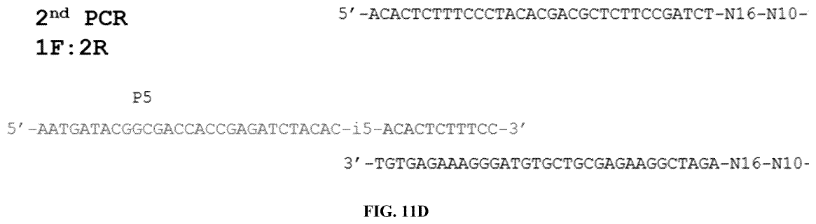

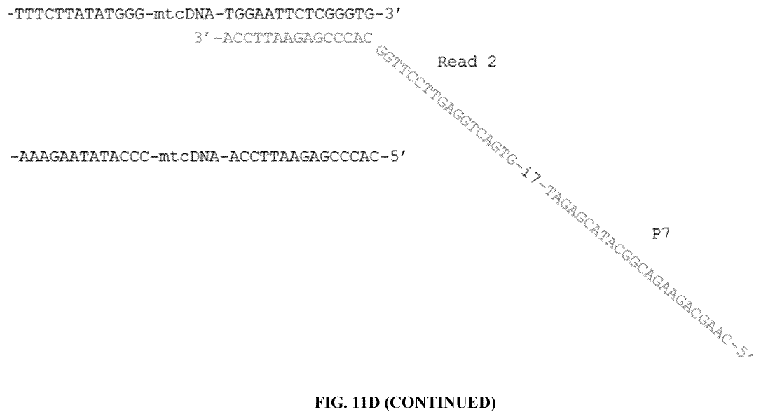

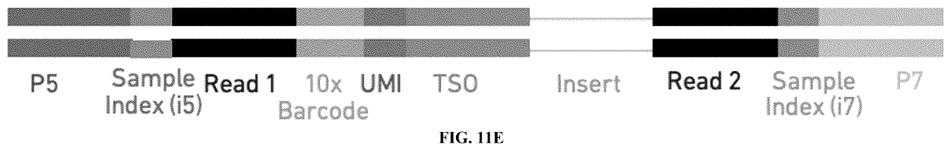

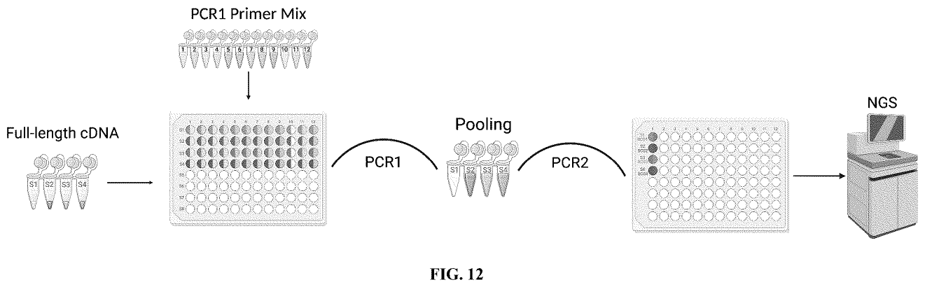

In various aspects, the present disclosure provides improved methods for the preparation of single-cell cDNA libraries for sequencing. In particular embodiments, the present disclosure provides improved methods for the preparation of single-cell mitochondrial cDNA libraries. The methods involve A) preparing a cDNA library containing polynucleotides containing cDNA from a single cell and B) preparing a sequencing library using said polynucleotides. An embodiment of a method for the preparation of single-cell cDNA libraries for sequencing is shown in FIGS. 11A-11E and 12.

In various embodiments, the methods of the disclosure involve A) preparing a cDNA library from a single cell, where the cDNA library contains polynucleotides containing cDNA prepared from mRNA from the single cell. In various embodiments, the polynucleotides contain the following regions in order from the 5′ end to the 3′ end, as depicted in FIG. 11B: a first partial Read 1 nucleotide sequence, a barcode sequence, a unique molecular identifier, a cDNA insert, and/or a Poly-dT reverse transcriptase (RT) primer. The cDNA library may be prepared using a Chromium Next GEM Single Cell 5′ Reagent Kit available from 10X Genomics. One of skill in the art is familiar with methods for preparation of cDNA from single cells using kits available from 10X Genomics (see, e.g., Caixia, et al., Current Genomics, 21:602-609 (2020)). In various embodiments, preparation of the cDNA library involves a Gel Beads-in-emulsion (GEM) method for the preparation of the cDNA libraries, where the GEM method involves combining gel beads with single cells obtained from a biological sample. The gel beads each have bound to their surface DNA oligomers (“Gel Bead Primers”) containing from the 5′ end to the 3′ end the first partial Read 1 nucleotide sequence (e.g., a partial Illumina RI sequence), a barcode, a unique molecular identifier (UMI), and a template switch oligo (TSO), where the oligomers are linked to the surface of the beads by their 5′ ends. In some embodiments, the first partial Read 1 sequence is from about or at least about 15, 16, 17, 18, 19, 20, 21, 22, 23, 24, or 25 nucleotides in length. In some embodiments, the first partial Read 1 sequence is no more than about 15, 16, 17, 18, 19, 20, 21, 22, 23, 24, or 25 nucleotides in length. In various embodiments, the Gel Bead Primers contain the following nucleotide sequence: 5′-CTACACGACGCTCTTCCGATCT-NNNNNNNNNNNNNNNN-NNNNNNNNNN-TTTCTTATATGGG-3′ (SEQ ID NO: 10), where the first partial Read 1 sequence is in bold, the first sequence of N's is a barcode, the second sequence of N′s is a unique molecular identifier, and the TSO sequence is underlined. Combining the gel beads with the single cells involves using a microfluidic device (e.g., a Chromium Next GEM Chip K available from 10X Genomics) to prepare an aqueous solution-in-water emulsion where each aqueous solution droplet within the emulsion contains a single cell, a gel bead, and reagents appropriate for the preparation of a cDNA library within each aqueous solution droplet, where the reagents in various embodiments include a primer containing the following nucleotide sequence: 5′-AAGCAGTGGTATCAACGCAGAGTACTTTTTTTTTTTTTTTTTTTTTTTTTTTTTTVN-3′ (SEQ ID NO: 11) and a reverse transcriptase. To achieve single-cell resolution, cells are delivered to the microfluidic device at an appropriate dilution such between 90% and 99% of aqueous solution droplets within the emulsion contain no cell, while the remainder of the aqueous solution droplets largely contain a single cell. The gel beads are dissolved upon preparation of the emulsion and the emulsion is subjected to appropriate conditions to allow the reactions depicted in FIG. 11A to take place yielding the cDNA libraries containing polynucleotides each having the sequence depicted in FIG. 11B. Each droplet in the emulsion contains reagents appropriate to prepare cDNA from mRNA (e.g., each droplet contains a reverse transcriptase enzyme). In some embodiments, each droplet contains a DNA polymerase. Non-limiting examples of DNA polymerases suitable for use in the methods of the disclosure include those familiar to one of skill in the art and suitable for use in the methods of the disclosure, such as those disclosed in Ordonez and Redrejo-Rodriguez, Int. J. Mol. Sci. 24:9331 (2023) doi: 10.3390/ijms24119331, the disclosure of which is incorporated herein by reference in its entirety for all purposes.

The methods of the disclosure further involve B) preparing sequencing libraries from the cDNA libraries prepared as described above. An embodiment of a method for preparing the sequencing libraries is shown in FIGS. 11C and 11D. The method involves two PCR reactions including a 1^{st} PCR and a 2^{nd} PCR. The 1^{st} and 2^{nd} PCR reaction involve amplifying sequences from the cDNA libraries using a suitable DNA polymerase (see e.g., Ordonez and Redrejo-ACTIVE Rodriguez, “DNA Polymerases for Whole Genome Amplification: Considerations and Future Directions,” Int. J. Mol. Sci. 24:9331 (2023) doi: 10.3390/ijms24119331, the disclosure of which is incorporated herein in its entirety for all purposes).

In various embodiments, the mass of the cDNA library polynucleotides used in the each 1^{st} PCR reaction(s) is about or at least about 0.5 ng, 1 ng, 1.5 ng, 2 ng, 2.5 ng, 3 ng, 3.5 ng, 4 ng, 4.5 ng, 5 ng, 6 ng, 7 ng, 8 ng, 9 ng, 10 ng, 11 ng, 12 ng, 13 ng, 14 ng, 15 ng, 16 ng, 17 ng, 18 ng, 19 ng, 20 ng, 25 ng, or 30 ng. In some embodiments, the mass of the cDNA library polynucleotides used in each 1^{st} PCR is less than about 1 ng, 1.5 ng, 2 ng, 2.5 ng, 3 ng, 3.5 ng, 4ng, 4.5 ng, 5 ng, 6 ng, 7 ng, 8 ng, 9 ng, 10 ng, 11 ng, 12 ng, 13 ng, 14 ng, 15 ng, 16 ng, 17 ng, 18 ng, 19 ng, 20 ng, 25 ng, or 30 ng.

In various embodiments, the primers used in the 1^{st} PCR reaction(s) contain a first forward primer containing in various embodiments the nucleotide sequence listed in Table 1 (i.e., a primer containing an Illumina R1 sequence) and one or more first reverse primers referred to as RNA sequencing primers (RSPs). In some cases, the first forward primer contains a Read 1 sequence comprising from 5′ to 3′, a second partial Read 1 sequence and the first partial Read 1 sequence. In some embodiments, the second partial Read 1 sequence is from about or at least about 5, 6, 7, 8, 9, 10, 11, 12, 13, 14, 15, 16, 17, 18, 19, or 20 nucleotides in length. In some embodiments, the first partial Read 1 sequence is no more than about 5, 6, 7, 8, 9, 10, 11, 12, 13, 14, 15, 16, 17, 18, 19, or 20 nucleotides in length. In some cases, the second partial Read 1 sequence is not complementary to nucleotides adjacent to (e.g., within 1, 2, 3, 4, 5, 6, 7, 8, 9, or 10 nucleotides of) a sequence complementary to the first partial Read 1 sequence in the cDNA library. In embodiments, the 1^{st} PCR reaction(s) involves a set of 1, 2, 3, 4, 5, 6, 7, 8, 9, 10, 11, 12, 13, 14, 15, 20, 25, or 30 1st PCR reactions, where each 1^{st} PCR reaction involves the use of a subset of a set of RSPs and the set of 1^{st} PCRs includes the use of the full set of RSPs. The RSPs each contain in order from the 5′ end to the 3′ end, a partial Read 2 nucleotide sequence (i.e., partial Illumina R2 sequence), such as CACCCGAGAATTCCA (SEQ ID NO: 3), and a targeting sequence complementary to a mitochondrial cDNA target site and. In some embodiments, the first partial Read 2 sequence is from about or at least about 10, 11, 12, 13, 14, 15, 16, 17, 18, 19, or 20 nucleotides in length. In some embodiments, the second partial Read 2 sequence is no more than about 10, 11, 12, 13, 14, 15, 16, 17, 18, 19, or 20 nucleotides in length. In various embodiments, the full set of RSPs is selected such that the RSPs are tiled across a mitochondrial transcriptome such that the sites targeted by the RSPs are spaced from one another by about or at least about 100 nt, 150 nt, 200 nt, 250 nt, 300 nt, 350 nt, 400 nt, 450 nt, or 500 nt. In some cases, the RSPs are selected such that they are tiled across a mitochondrial transcriptome such that the sites targeted by the RSPs are spaced from one another by no greater than about 100 nt, 150 nt, 200 nt, 250 nt, 300 nt, 350 nt, 400 nt, 450 nt, or 500 nt. Non-limiting examples of RSPs are listed in Table 2. In various embodiments, each of the set of 1^{st} PCR reactions involves the use of about or at least about 1, 2, 3, 4, 5, 6, 7, 8, 9, 10, 15, or 25 RSPs as first reverse primers. In various embodiments, each of the set of 1^{st} PCR reactions involves the use less than about 2, 3, 4, 5, 6, 7, 8, 9, 10, 15, or 25 RSPs as first reverse primers. In some cases, the 1^{st} PCR involves a set of 12 1^{st} PCR reactions, where each of the 1^{st} PCR reactions involves the use of the first forward primer (i.e., “Read 1 primer”) listed in Table 1 and where each of the 12 1^{st} PCR reactions involves using as first reverse primers one of the 12 RSP mixtures indicated listed in Table 2. In various embodiments, each RSP mixture contains an equimolar amount of each RSP reverse primer and all of the RSP mixtures contain the same molarity of each RSP primer. In various embodiments, each RSP mixture contains primers corresponding to amplicons having lengths that have similar lengths that differ from one another by no more than about 25 bp, 50 bp, 75 bp, 100 bp, 150 bp, 200 bp, 250 bp, or 500 bp. It can be advantageous to select the RSP primers in each RSP mixture such that no RSP mixture contains more than 1 RSP primer targeting the same cDNA sequence.

In various embodiments, the 1^{st} PCR reaction(s) is carried out using a molar ratio of first forward to first reverse (F:R) primers that is about 1:2, 1:25, 1:3, 1:4, 1:5, 1:6, 1:7, 1:8, 1:9, or 1:10. In some cases F:R is about 1:5. In some cases the F:R ratio is about 1:2.5. In various embodiments, the concentration of each RSP primer used in the 1^{st} PCR reaction(s) is about or at least about 0.01 μM, 0.02 μM, 0.03 μM, 0.04 μM, 0.05 μM, 0.06 μM, 0.07 μM, 0.08 μM, 0.09 μM, 0.1 μM, 0.15 μM, 0.2 μM, 0.25 μM, 0.3 μM, 0.35 μM, 0.4 μM, 0.45 μM, or 0.5 μ.

In various embodiments, the 1^{st} PCR reaction(s) is carried out using an annealing temperature of between about 59° C. and 61° C., of between about 58° C. and 62° C., of between about 55° C. and 65° C., or of about 60° C. In some cases the 1^{st} PCR reaction(s) involves about or at least about 5, 6, 7, 8, 9, 10, 11, 12, 13, 14, 15, or 20 primer extension cycles. In some cases the 1^{st} PCR reaction(s) involves no more than about 5, 6, 7, 8, 9, 10, 11, 12, 13, 14, 15, or 20 primer extension cycles. In particular embodiments, the 1^{st} PCR involves about 13 primer extension cycles.

Following the 1^{st} PCR reaction(s), the amplicons resulting from said reaction are pooled and cleaned. The cleaning can involve the use of solid-phase reversible immobilization (SPRI) beads (such as AMPure XP beads available from Beckman Coulter). It can be advantageous to pool the reaction products from the 12 1^{st} PCR reactions described above (see also Table 2) such that the amplicons from the 12 1^{st} PCR reactions are pooled at the following ratios, where the ratios may be volumetric (i.e., volume of the reaction mixture), molar (i.e., total molar amounts of amplicons from each reaction), or mass (i.e., total mass of the amplicons from reaction) ratios of: 40 of each of the amplicon mixtures corresponding to primer mixes 1-9 to 12 of the amplicon mixture corresponding to primer mix 10, to 8 of each of the amplicon mixtures corresponding to primer mixes 11 and 12 (i.e., the ratio 40:40:40:40:40:40:40:40:40:12:8:8). Cleaning the pooled amplicons from the 1^{st} PCR reactions(s) involves eluting the amplicons from the SPRI beads using water (e.g., nuclease free water) or an aqueous solution with an elution volume of the water or aqueous solution to yield an eluate. In various embodiments, the elution volume is about or at least about 50 μL, 75 μL, 100 μL, 125 μL, 150 μL, 175 μL, 200 μL, 225 μL, 250 μL, 275 μL, 300 μL, 325 μL, 350 μL, 375 μL, 400 μL, 425 μL, 450 μL, 475 μL, or 500 μL. The elution volume may be less than about 50 μL, 75 L, 100 μL, 125 μL, 150 μL, 175 μL, 200 μL, 225 μL, 250 μL, 275 μL, 300 μL, 325 μL, 350 μL, 375 μL, 400 μL, 425 μL, 450 μL, 475 μL, or 500 μL. In various embodiments, about 5 μL, 6 μL, 7 μL, 8 μL, 9 μL, 10 μL, 11 μL, 12 μL, 13 μL, 14 μL or 15 μL of the eluate is used as an input for the 2^{nd} PCR reaction.

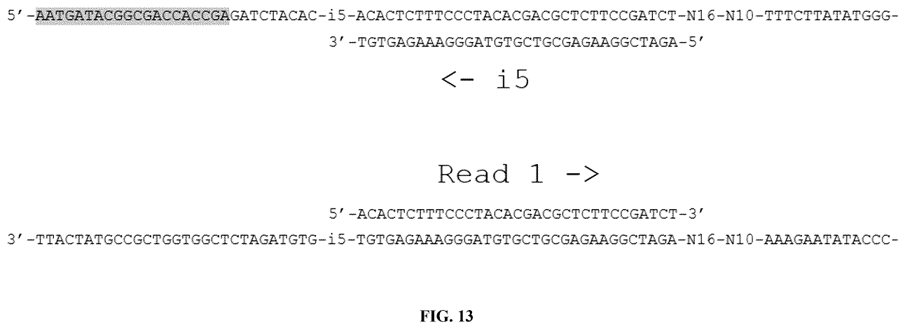

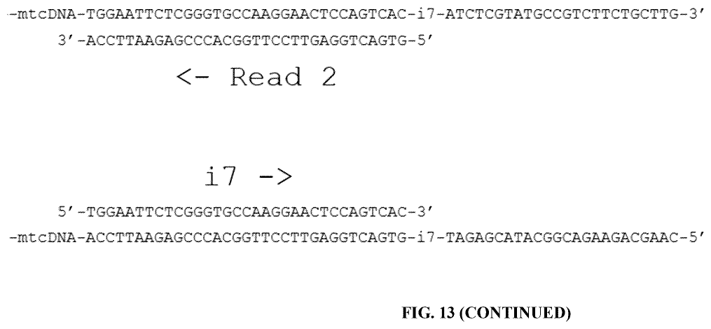

In various embodiments, the 2^{nd} PCR reaction involves the use of a second forward primer containing the nucleotide sequence 5′-AATGATACGGCGACCACCGAGATCTACAC-15-ACACTCTTTCC-3′ (SEQ ID NOS 12-13, respectively) and a second reverse primer containing the nucleotide sequence 5′-GTGACTGGAGTTCCTTGGCACCCGAGAATTCCA-17-CAAGCAGAAGACGGCATACGAGAT-3′ (SEQ ID NOS 14-15, respectively), where i5 and i7 indicate sample indexing primers (see, e.g., the sample indexing PCR primer sequences listed in Table 3 providing non-limiting examples of such forward and reverse primers). The second forward primer contains, in order from the 5′ end to the 3′ end, a P5 sequence, a first indexing sequence (e.g., an i5 indexing sequence), and a the second partial Read 1 sequence (e.g., partial Illumina R1 sequence), and the second reverse primer contains, in order from the 5′ end to the 3′ end, a P7 sequence, a second indexing sequence (e.g., an i7 indexing sequence), and a Read 2sequence (e.g., Illumina R2 sequence) containing from 5′ to 3′ a second partial Read 2 sequence and the first partial Read 2 sequence, where in various embodiments the P5 and P7 sequences are priming sites used in an Illumina sequencer. The 2^{nd} PCR is carried out to add the i5 and i7 indices and the P5 and P7 sequences to the sequencing libraries are produced using the 2^{nd} PCR reaction (see, FIG. 11E providing a schematic diagram showing the sequence of the polynucleotides of the sequencing libraries). The use of i5, i7, P5, P7, R1, and R2 sequences in sequencing libraries prepared for sequencing using Illumina next-generation sequencing are familiar to one of skill in the art (see, e.g., Glenn, et al., bioRxiv 049114, doi: 10.1101/049114). The amplicons produced by the 2^{nd} PCR reaction constitute a sequencing library containing in order from the 5′ end to the 3′ end the following regions: P5, i5, Read 1, barcode, UMI, TSO, cDNA insert (e.g., mitochondrial cDNA insert), Read 2, i7, and P7. The sequencing libraries are suitable for use in sequencing the cDNA inserts by way of next-generation sequencing, such as Illumina sequencing (e.g., Illumina sequencing carried out using a NovaSeq™M 6000 v1.5 Reagent Kit (e.g., as shown schematically in FIG. 13).

The first and/or second indexing sequence may independently be about or at least about 2, 3, 4, 5, 6, 7, 8, 9, 10, 11, 12, 13, 14, or 15 nucleotides in length. The first and/or second indexing sequence may independently be less than about 3, 4, 5, 6, 7, 8, 9, 10, 11, 12, 13, 14, or 15 nucleotides in length.

The barcode and/or UMI sequence may independently be about or at least about 2, 3, 4, 5, 6, 7, 8, 9, 10, 11, 12, 13, 14, 15, 16, 17, 18, 19, or 20 nucleotides in length. The barcode and/or UMI may independently be less than about 3, 4, 5, 6, 7, 8, 9, 10, 11, 12, 13, 14, or 15 nucleotides in length.

In some embodiments, the second partial Read 2 sequence is from about or at least about 15, 16, 17, 18, 19, 20, 21, 22, 23, 24, or 25 nucleotides in length. In some embodiments, the second partial Read 2 sequence is no more than about 15, 16, 17, 18, 19, 20, 21, 22, 23, 24, or 25 nucleotides in length. In some cases, the second partial Read 2 sequence is not complementary to nucleotides adjacent to (e.g., within 1, 2, 3, 4, 5, 6, 7, 8, 9, or 10 nucleotides of) a sequence complementary to the first partial Read 2 sequence in the cDNA library.

In some embodiments, the P7 sequence is from about or at least about 20, 21, 22, 23, 24, 25, 26, 27, 28, 29, 30, 31, 32, 33, 34, 35, 36, 37, 38, 39, or 40 nucleotides in length. In some embodiments, the P7 sequence is no more than about 20, 21, 22, 23, 24, 25, 26, 27, 28, 29, 30, 31, 32, 33, 34, 35, 36, 37, 38, 39, or 40 nucleotides in length.

In some embodiments, the P5 sequence is from about or at least about 20, 21, 22, 23, 24, 25, 26, 27, 28, 29, 30, 31, 32, 33, 34, 35, 36, 37, 38, 39, or 40 nucleotides in length. In some embodiments, the P5 sequence is no more than about 20, 21, 22, 23, 24, 25, 26, 27, 28, 29, 30, 31, 32, 33, 34, 35, 36, 37, 38, 39, or 40 nucleotides in length.

In various embodiments, the first forward primer, the first reverse primer, the second reverse primer, and the second forward primer do not form primer dimers that prevent the production of amplicons during a PCR reaction containing two or more of said first forward primer, first reverse primer, second reverse primer, and second forward primer.

In some cases, it can be advantageous in the 2^{nd} PCR reaction that the molar ratio of second forward to second reverse primer (F:R) be about 1:2, 1:3, 1:4, or 1:5. In various embodiments, the F:R ratio is 1:5. In some cases, the concentration of the second reverse primer is about, or at least about 0.45 μM, 0.90 μM, 1.35 μM, 1.8 μM, 2.25 M, 2.7 μM, 3.15 μM, 3.6 μM, 4.05 μM, 4.95 μM, 5.4 μM, or 5.85 μM. In some cases, the concentration of the second reverse primer is less than about 0.90 μM, 1.35 μM, 1.8 μM, 2.25 μM, 2.7 μM, 3.15 μM, 3.6 μM, 4.05 μM, 4.95 μM, 5.4 μM, or 5.85 μM.

In various embodiments, the mass of the cDNA library polynucleotides used in the 2nd PCR reaction is about or at least about 0.5 ng, 1 ng, 1.5 ng, 2 ng, 2.5 ng, 3 ng, 3.5 ng, 4 ng, 4.5 ng, 5 ng, 6 ng, 7 ng, 8 ng, 9 ng, 10 ng, 11 ng, 12 ng, 13 ng, 14 ng, 15 ng, 16 ng, 17 ng, 18 ng, 19 ng, 20 ng, 25 ng, or 30 ng. In some embodiments, the mass of the cDNA library polynucleotides used in the 2^{nd} PCR reaction is less than about 1 ng, 1.5 ng, 2 ng, 2.5 ng, 3 ng, 3.5 ng, 4 ng, 4.5 ng, 5 ng, 6 ng, 7 ng, 8 ng, 9 ng, 10 ng, 11 ng, 12 ng, 13 ng, 14 ng, 15 ng, 16 ng, 17 ng, 18 ng, 19 ng, 20 ng, 25 ng, or 30 ng.

In various embodiments, the 2^{nd} PCR reaction is carried out using an annealing temperature of between about 53° C. and 55° C., of between about 52° C. and 57° C., of between about 50° C. and 60° C., or of about 54° C. In some cases the 2^{nd} PCR reaction involves a denaturation step (e.g., an incubation at a temperature of about or at least about 85° C., 90° C., or 95° C.) that is about or at least about 35 s, 40 s, 45 s, 50 s, or 55 s. In some cases the 2^{nd} PCR reaction involves a denaturation step that is less than about 35 s, 40 s, 45 s, 50 s, or 55 s. The denaturation step may be 45 s.

In various embodiments, the above-described method provides sequence libraries where at least about 15%, 20%, 25%, 30%, 35%, 40%, 45%, 50%, 55%, 60%, 65%, 70%, 75%, 80%, 85%, 90%, or more of the polynucleotides in the library are sequenceable.

Methods for analyzing nucleic acids from single cells are known in the art and described for example in U.S. Pat. No. 9,102,980 and in U.S. Patent Application Publication No. 2021/0032702 A1, the disclosures of which are incorporated herein by reference in their entirety for all purposes.

In various embodiments, one or more of the sequences provided herein may contain as many as about 1, 2, 3, 4, 5, 6, 7, 8, 9, or 10 nucleotide alterations.

In various embodiments, the P5 and P7 sequences are each complementary to oligonucleotides bound to a surface of a flow cell used in next for generation sequencing of the cDNA libraries produced by the methods provided herein. In some embodiments, the oligonucleotides bound to the flow cell are extended in a DNA synthesis reaction using a sequencing library polynucleotide as a template for the DNA synthesis. In some embodiments, the Read 1 and Read 2 sequences are complementary to sequencing primers used for next generation sequencing of the cDNA libraries produced by the methods provided herein. In various embodiments, the P5 and P7 sequences are complementary to primers used in a bridge amplification reaction carried out during next generation sequencing.

### Barcodes and Unique Molecular Identifiers

The sequencing libraries of the present disclosure may contain unique molecular identifiers (UMIs) (see, e.g., Kivioja et al., 2012, Nat. Methods. 9 (1): 72-4 and Islam et al., 2014, Nat. Methods. 11 (2): 163-6) and/or unique barcodes (BCs). A barcode herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment.

Barcoding may be performed based on any of the compositions or methods available to one of skill in the art, such as those disclosed in International Patent Publication No. WO 2014/047561 A1, the disclosure of which is incorporated herein by reference in its entirety for all purposes. In certain embodiments, barcoding uses an error correcting scheme (see, T. K. Moon, Error Correction Coding: Mathematical Methods and Algorithms (Wiley, New York, ed. 1, 2005)). In embodiments of the disclosure, amplified sequences from single cells may be sequenced together and resolved based on the barcode associated with each cell.

In some embodiments, sequence libraries contain unique molecular identifiers (UMIs). A Unique Molecular Identifier may be a short (usually 4-10 bp) random barcode added to transcripts during reverse-transcription. They may enable sequencing reads to be assigned to individual transcript molecules and thereby facilitate in some embodiments the removal of amplification noise and biases from sequencing data. The UMIs may also be used to determine the number of transcripts that gave rise to an amplified product.

### Sequencing Methods

Sequencing of the sequencing libraries prepared according to the methods of the disclosure may be performed on any high-throughput platform. Methods of sequencing oligonucleotides and nucleic acids are well known in the art (see, e.g., WO 93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); No. WO 1993/021340A1; Metzker et al., Nucl. Acids Res. 22:4259 (1994); Jones, Biotechniques 22:938 (1997); Ronaghi et al., Anal. Biochem. 242:84 (1996); Ronaghi et al.,

Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91 (13): 5740 (1994), all of which are expressly incorporated by reference).

The sequencing of a polynucleotide can be carried out using any suitable commercially available sequencing technology. In embodiments, the sequencing of a polynucleotide is carried out using a chain termination method of DNA sequencing (e.g., Sanger sequencing). In some embodiments, commercially available sequencing technology is a next-generation sequencing (NGS) technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLID sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing.

Illumina sequencing is a sequence by synthesis method for sequencing DNA. Typically, Illumina sequencing is carried out in an acrylamide-coated glass flow cell having oligonucleotides coating the bottom of the cell serving as a solid support to hold polynucleotides of a sequencing library in place during sequencing.

In embodiments, the disclosure involves the use of RNA-seq. In connection with this technology, mRNA derived from samples is reverse transcribed to complementary DNA (cDNA), which is then converted to a sequencing library. This library is sequenced in a sequencing instrument and the output is then sorted into quantitative lists of transcripts that are present in each sample. The data output of such an RNA-seq experiment will include most or all of the mRNAs present in the sample(s) at the time of collection. In some embodiments, the disclosure involves the use of single cell RNA-seq (scRNAseq) involving preparation and subsequent sequencing of cDNA libraries from single cells.

In various embodiments, sequencing of the mitochondrial cDNA sequencing libraries prepared according to the methods of the disclosure results in a mean or median sequence coverage of at least 1, 2, 3, 4, 5, 6, 7, 8, 9, 10, 11, 12, 13, 14 or 15 mitochondrial transcripts that is about, or at least about 10×, 20×, 30×, 40×, 50×, 60×, 70×, 80×, 90×, or 100×.

### Types of Samples

This disclosure provides methods to extract and sequence a polynucleotide present in a sample. In one embodiment, the samples are biological samples generally derived from a human subject, such as a subject having a neoplasia or a healthy subject or a subject with autoimmune disease or a subject with cardiovascular disease or another type of disease associated with a clone of cells. In embodiments, the sample is derived from a neoplasia (e.g., cancer, tumor). In embodiments, the sample is a biopsy (e.g., needle biopsy, tissue biopsy). In embodiments, the sample is a bodily fluid (such as ascites, blood, bone marrow, plasma, pleural fluid, serum, cerebrospinal fluid, phlegm, saliva, stool, urine, semen, prostate fluid, breast milk, or tears, or tissue sample (e.g. a tissue sample obtained by biopsy). In a further embodiment, the samples are biological samples derived from an animal, such as a bodily fluid (such as blood, bone marrow, cerebrospinal fluid, phlegm, saliva, or urine) or tissue sample (e.g., a tissue sample obtained by biopsy). In still another embodiment, the samples are biological samples from in vitro sources (such as cell culture medium). In some instances, the sample is a cancer biopsy or a tumor section.

### Characterization of Cells

In various aspects, the methods of the disclosure involve determining, based upon single-cell mitochondrial RNA sequence data collected by way of the methods of the disclosure, a lineage or clonal structure for a tumor or cancer.

In certain embodiments, such as in multicellular organisms, the progeny of single dividing cells cannot be followed and a cell lineage or clonal structure is inferred retrospectively (e.g., after cell division has already occurred). The present disclosure provides for improved methods for inferring a cell lineage or clonal structure by detecting somatic mutations, specifically somatic mutations that occur in the mitochondrial genome. Determination of somatic mutations (e.g., including mitochondrial mutations, such as single nucleotide variants (SNVs)) allows cells derived from a tissue or tumor to be clustered based on the mutations. The clustering provides for identifying cells that share a common lineage.

Clustering or cluster analysis involves grouping a set of objects (e.g., cells) in such a way that objects in the same group (called a cluster) are more similar (in some sense) to each other than to those in other groups (clusters). It is a task of exploratory data mining, and a common technique for statistical data analysis, used in many fields, including machine learning, pattern recognition, image analysis, information retrieval, bioinformatics, data compression, and computer graphics. Non-limiting examples of techniques for clustering sequence data include hierarchical clustering, graph-based clustering, non-negative matrix factorization, Leiden or Louvain clustering, mixture models, k-means, ensemble learning, neural networks and density-based clustering. Clustering may involve agglomerative hierarchical clustering. Various methods for clustering single-cell RNA-sequencing data are familiar to one of skill in the art (see, e.g., Petegrosso, et al. Brief Bioinform 15:1209-1223 (2020), doi: 10.1093/bib/bbz063).

In certain embodiments, single cells are clustered by hierarchical clustering using somatic mutations (e.g., single nucleotide variants (SNVs)).

### Cell States

In certain embodiments, expression of one or more genes in cells falling within the clusters identified as described herein is determined. The gene expression can be mapped onto specific cell lineages or clonal structures.

In certain embodiments, the cell state is determined by analyzing the transcriptomic sequencing data generated according to the methods of the disclosure. Single cell RNA sequencing allows for detecting mitochondrial genome mutations in the transcribed mitochondrial RNA as well as levels of gene expression in the cells from which the mitochondrial DNA was collected.

### Lineages and Clonal Populations in Tissues

In certain embodiments, the single cells analyzed according to the methods of the disclosure may be cells from a tissue, such as a tumor or biopsy, or the cells may be neoplastic cells.

The present disclosure provides for a method of characterizing neoplastic cells from a subject. The methods may involve identifying genomic changes in clonal populations and/or determining clonal populations of cells. The present disclosure allows for improved determination of clonal populations and subpopulations.

### Clonal Populations

In another aspect, the present disclosure provides for a method of identifying and characterizing clonal populations of cells in a biological sample obtained from a subject having a tumor or cancer or a healthy subject or a subject with another disease or at risk of developing a disease. In some instances, the cells are neoplastic cells, such as multiple myeloma cells. In certain embodiments, clonal populations and/or subpopulations of cells are identified based on the presence of copy number variants (CNVs) or mitochondrial single nucleotide variants (SNVs) identified using single-cell RNA sequencing data gathered according to the methods of the disclosure.

In various aspects, the methods of the disclosure involve determining, based upon single-cell mitochondrial RNA sequence data collected by way of the methods of the disclosure, clonal expansions of cells. In embodiments, the identification of such clonal expansions may facilitate selection of a treatment to be administered to a subject (e.g., administration of a therapeutically effective amount of an agent to reduce or eliminate expansion of the cells or reduce or eliminate the number of the cells in a subject).

In various embodiments, the methods of the disclosure involve the identification of copy number variants or somatic mutations in a clonal population of cells.

With the advent of next-generation sequencing which enables the interrogation of human tissues at great depth, mutant clones have been discovered in otherwise healthy individuals demonstrating that the mere presence of genetically altered clonal expansions does not necessarily constitute a malignancy. In fact, as humans age, the incidence of these clonal expansions increases. One example of this phenomenon is the condition called Clonal Hematopoiesis of Indeterminate Potential or CHIP, where individuals present with clonal expansions of hematopoietic stem cells, which can differentiate into mature blood cells of the myeloid or lymphoid lineage. Individuals with CHIP are at higher risk to develop hematological malignancies, but importantly they are additionally at risk for non-cancerous disease, such as cardiovascular disease, autoimmune disease and even aging-associated conditions that require surgery such as degenerative arthritis requiring hip arthroplasty (see, e.g., N Engl J Med 2017, 377:111-121, DOI: 10.1056/NEJMoa1701719; Blood 2021 Nov. 4; 138 (18): 1727-1732. Doi: 10.1182/blood.2020010163; and the disclosure of which is incorporated herein by reference in its entirety for all purposes). Data from mouse models has demonstrated that CHIP clones may lead to increased systemic inflammation, which in turn can cause or exacerbate disease. Recent data supports the existence of mutant T cell clones in patients with refractory celiac disease (see, e.g., medRxiv 2024.03.17. 24304320, doi: 10.1101/2024.03.17.24304320, the disclosure of which is incorporated herein in its entirety by reference in its entirety for all purposes). The implication of these clones in malignant and non-malignant disease raises the possibility that clone-targeting treatment may eradicate or delay the progression of some of these malignant and non-malignant diseases. Therefore, sensitive methods to detect the presence of clones and to characterize their genomic and transcriptomic profile, such as the methods provided herein for single cell sequencing of mitochondrial RNA (scMito-seq), may be helpful in studying these phenomena at the research level and following them in clinical practice. Somatic mutations associated with cancer may include mutations associated with prognosis, treatment, and/or resistance to treatment. Mutations associated across the spectrum of human cancer types have been identified (e.g., Hodis E. et al., Cell. (2012) July 20; 150 (2): 251-63; and Vogelstein, et al., Science (2013) March 29: Vol. 339, Issue 6127, pp. 1546-1558). A directory of cancer mutations, including gene specific mutations may be found at cancer.sanger.ac.uk/cosmic, the Catalogue of Somatic Mutations in Cancer (COSMIC) (Forbes, et al.; COSMIC: somatic cancer genetics at high-resolution. Nucleic Acids Res 2017; 45 (D1): D777-D783. doi: 10.1093/nar/gkw1121). In certain embodiments, any of these known mutations may be detected depending on the cancer type. A biological sample may be collected from a tumor before, during, or subsequent to a cancer treatment. Sequencing of mitochondrial RNA from said sample may be used to characterize a cancer in a subject, to monitor a treatment for the cancer, or to monitor the subject for relapse of the cancer. The methods of the disclosure may involve obtaining a sample from a subject having cancer after administration of a cancer treatment to the subject and comparing the presence of clonal and/or subclonal populations before and after treatment, where the comparison may allow for the identification of clonal populations of cells sensitive or resistant to the treatment. The method may involve determining single nucleotide variants (SNVs), copy number variants (CNVs), clonal populations of cells, and/or subclonal populations of cells at at least one time point after administration of a therapy. The at least one time point may be a week, a month, a year, two years, three years, or five years after initiation of a therapy. The time point may be after a relapse in the disease is detected. Relapse may be any recurrence of symptoms of a disease after a period of improvement. Time points may be taken at any point after the initial treatment of the disease and includes time points following a change to the treatment or after the treatment has been completed.

The cancer treatment may be selected from the group consisting of chemotherapy, radiation therapy, immunotherapy, targeted therapy and combinations thereof. Methods for treating cancers include those familiar to one of skill in the art (see, e.g., Urruticoechea, A. et al., “Recent Advances in Cancer Therapy: An Overview,” Current Pharmaceutical Design, 16:3-10 (2010), and Bidram, E., et al. “A concise review on cancer treatment methods and delivery systems,” Journal of Drug Delivery Science and Technology, 54:101350 (2019), doi: 10.1016/j.jddst.2019.101350, the disclosure of which are incorporated herein by reference in their entireties for all purposes).

Computer Systems

The present disclosure also relates to a computer system involved in carrying out the methods of the disclosure (e.g., methods to characterize tumor or cancer cells).

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be, but is not limited to, an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules, and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine-readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a

PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user.

The computer can include appropriate software for receiving user instructions, either in the form of user input into a set of parameter fields, e.g., in a GUI, or in the form of preprogrammed instructions, e.g., preprogrammed for a variety of different specific operations. A computer can transform data into various formats for display. A graphical presentation of the results of a calculation can be displayed on a monitor, display, or other visualizable medium (e.g., a printout). In some embodiments, data or the results of a calculation may be presented in an auditory form.

In aspects, software used to analyze the data can include code that applies an algorithm to the analysis of the results. The software also can also use input data (e.g., sequence data or biochip data) to characterize cHL or PMBL.

### Kits

The disclosure also provides kits for use in single-cell sequencing of mitochondrial RNA and/or characterizing cells from a biological sample. Such kits may include enzymes (e.g., reverse transcriptase, DNA polymerase), sequencing primers, and/or forward and reverse primers of the disclosure. Kits of the instant disclosure may include one or more containers comprising an agent for use in the methods of the disclosure. In some embodiments, the kits further include instructions for use in accordance with the methods of this disclosure. In some embodiments, these instructions comprise a description of use of an agent(s) of the kit in single-cell sequencing of mitochondrial RNA and/or use of the agent(s) in characterization of cells from a biological sample. The kit may further comprise a description of how to analyze and/or interpret data obtained through the use of methods of the present disclosure. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein. The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the embodiments of the disclosure. Particularly useful techniques for specific embodiments will be discussed in the sections that follow.

The following examples are put forth so as to provide those of ordinary skill in the art with a complete disclosure and description of how to make and use an assay, screening, and therapeutic methods, and are not intended to limit the scope of what the inventors regard as their invention.

### EXAMPLES

### Example 1: Targeted 5′-End Single-Cell RNA Sequencing of Mitochondrial RNA

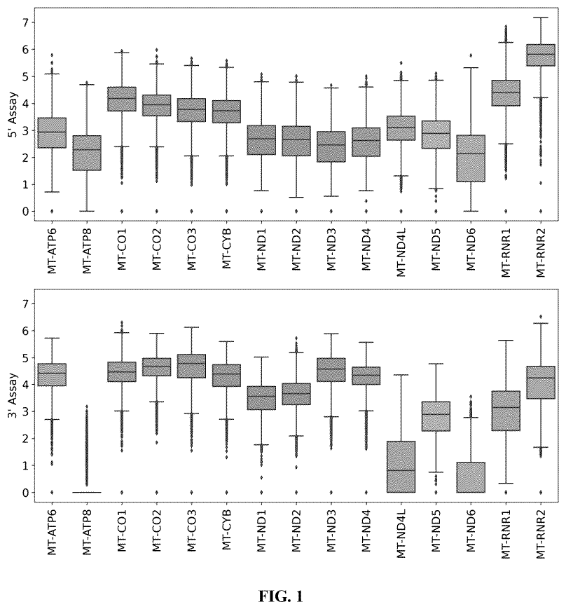

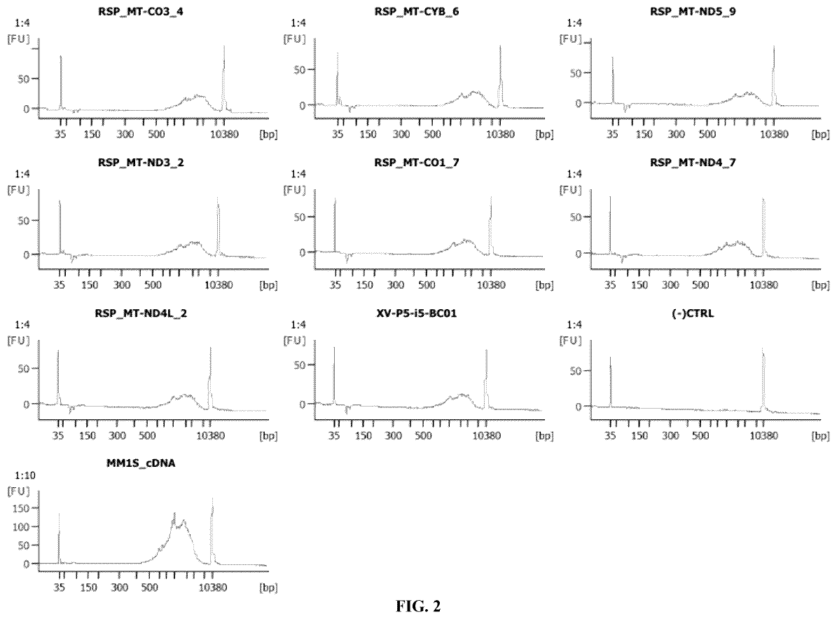

Experiments were undertaken to develop a method for 5′-end single-cell RNA sequencing of mitochondrial RNA involving targeted amplification of mitochondrial transcripts using primers (e.g., a Read 1 primer sequence, RNA sequencing primers (RSPs), and sample indexing PCR primers) and a methodology compatible with a Chromium 5′ Single Cell Gene Expression kit for single-cell libraries available from 10x Genomics. One consideration that motivated the development of a method for 5′-end single-cell RNA sequencing of mitochondrial RNA was that several mitochondrial transcripts, such as those expressed from the mitochondrial genes MT-ATP8, MT-ND4L, and MT-ND6, are not captured well using 3′-end single-cell RNA sequencing but are captured well using 5′-end single-cell RNA sequencing (see, e.g., FIG. 1). The method involves two steps of polymerase chain reaction (PCR) carried out using primers that bind to the mitochondrial transcriptome as well as primers that bind to classic Illumina read sequences and aid in maintaining the cell barcodes and providing sample indices to enable library multiplexing for next-generation sequencing, such as on an Illumina short-read sequencer. The libraries described herein were sequenced by Illumina next-generation sequencing using a NovaSeq™ 6000 v1.5 Reagent Kit (see FIG. 13).

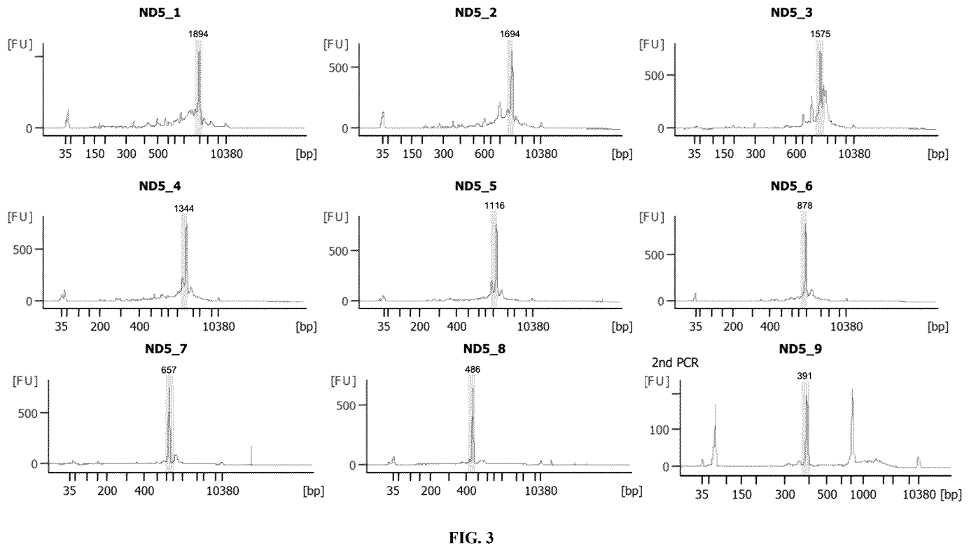

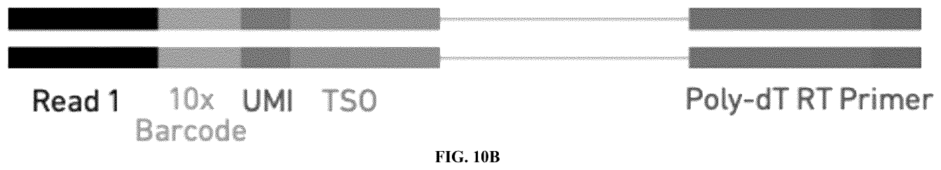

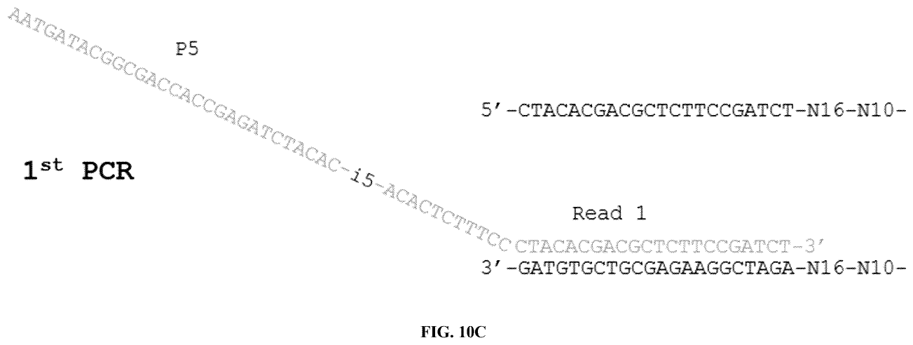

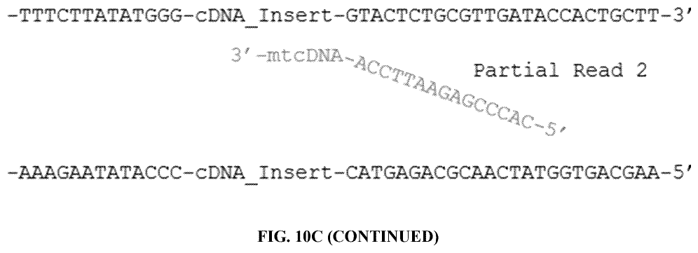

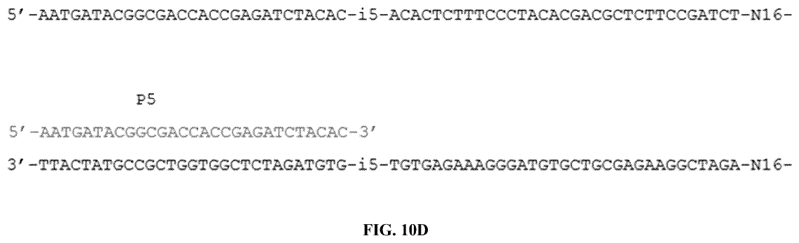

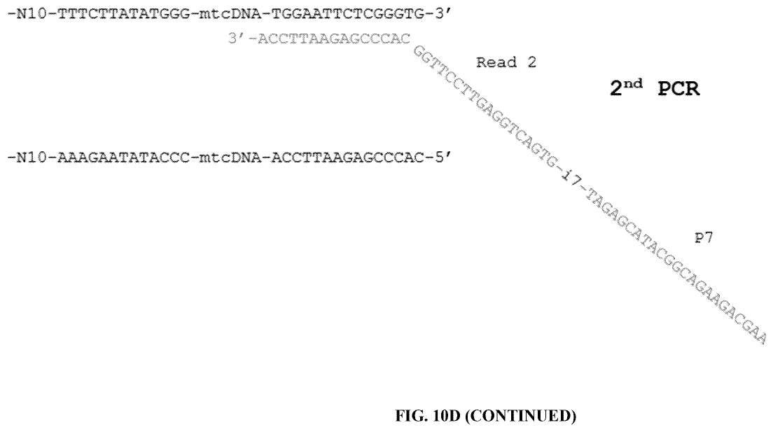

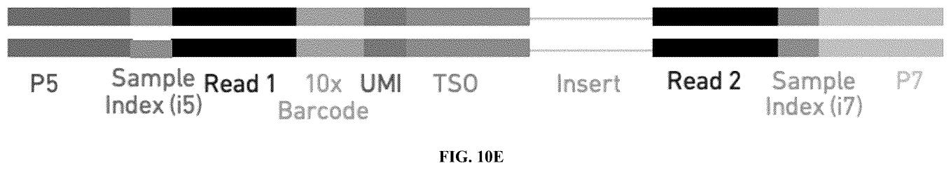

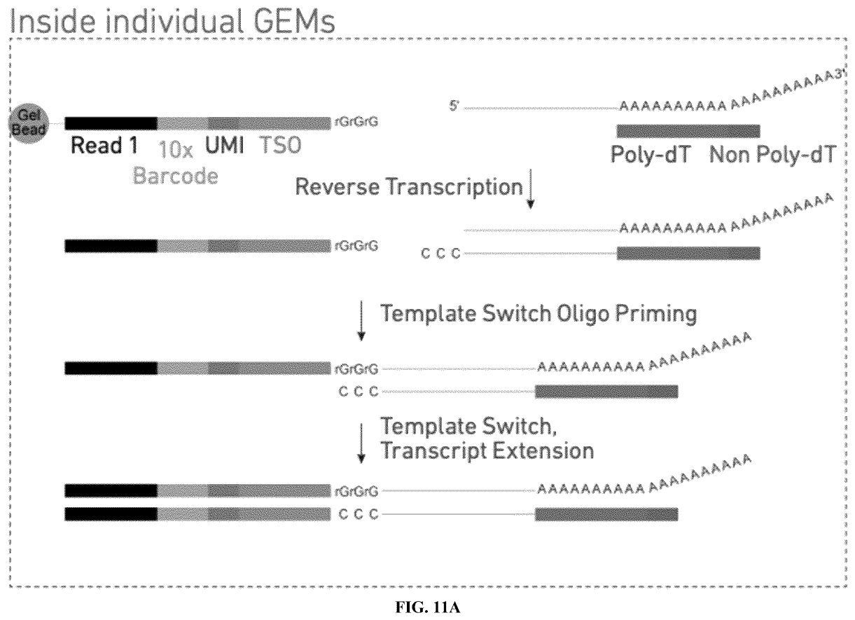

As a first step in developing the method, the 67 primers of Table 2 were designed to cover the majority of the mitochondrial transcriptome through tiling, where the sites targeted by each primer were spaced by about 250 bp from one another, which is equivalent to the sequence read length obtainable through Illumina sequencing. An experiment was undertaken to use the primers in the method described in FIGS. 10A-10E in an effort to reproduce the 3′-end single-cell RNA sequencing results presented in Miller, et al. Nat Biotechnol, 40:1030-1034 (2022), the disclosure of which is incorporated herein by reference in its entirety for all purposes, in the context of 5′-end single-cell RNA sequencing. In brief, barcoded full-length cDNA (FIG. 10B) was prepared using 10X Genomics' Chromium Single Cell 5′ Gene Expression Kit, as shown in FIG. 10A, and the full-length cDNA was then amplified using two PCR reactions in tandem (see FIGS. 10C and 10D) to yield libraries for sequencing (FIG. 10E). The libraries prepared by this method were unable to be sequenced because the method led to non-specific amplification of the barcoded cDNA (FIG. 2).

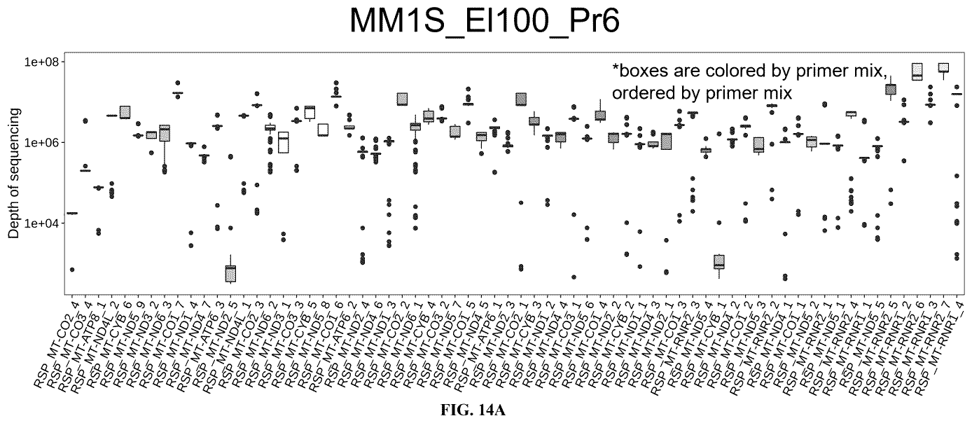

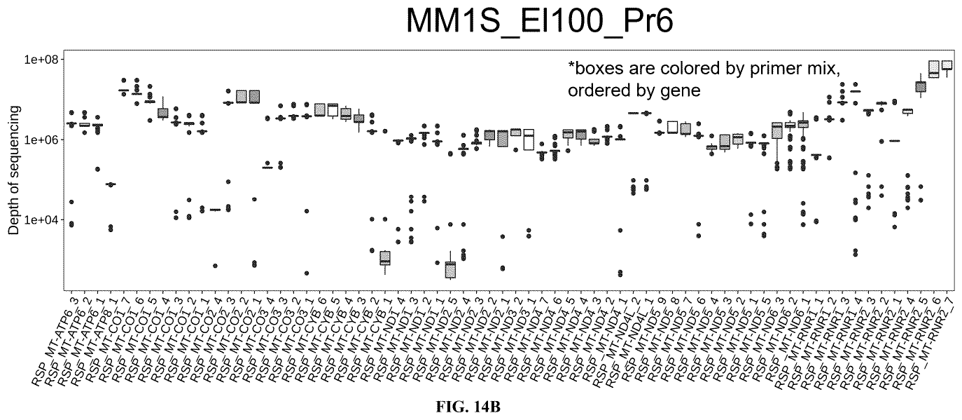

Experiments were next undertaken to modify the method depicted in FIGS. 10A-10E to allow for specific amplification of mitochondrial cDNA (FIG. 11A) prepared using 10X Genomics' Chromium Single Cell 5′ Gene Expression Kit, as shown in FIG. 11B. To achieve such specific amplification, the method depicted in FIGS. 10A-10E was modified by i) removing the P5 and i5 sequences from the forward primer depicted on the left of FIG. 10C to yield the forward primer depicted on the left in FIG. 11C and provided in Table 1 for use in the 1^{st} PCR reaction, ii) changing the ratio of forward (F) to reverse (R) (F:R) from 10:1 in the 1^{st} PCR reaction to 1:2.5 and the F:R ratio in 2^{nd} PCR reaction from 10:1 to 1:5, and iii) altering the thermal cycler conditions used during the 1^{st} PCR such that primer extension was carried out using an annealing temperature of 60° C. rather than 65° C. and the number of primer extension cycles was increased to 13 from 6. All 67 primers of Table 2 were evaluated separately to confirm specificity of amplification (see, e.g., FIG. 3). The method including the previously-described modifications and depicted in FIGS. 11A-11E led to the specific amplification of target mitochondrial cDNA amplicons and yielded libraries (FIG. 11E) that were suitable for sequencing because of, for example, insufficient yields of library amplicons. All primers of Table 2 performed well in the method (FIGS. 14A and 14B), and there was an association between amplicon length and coverage in CO1, but not in other genes.

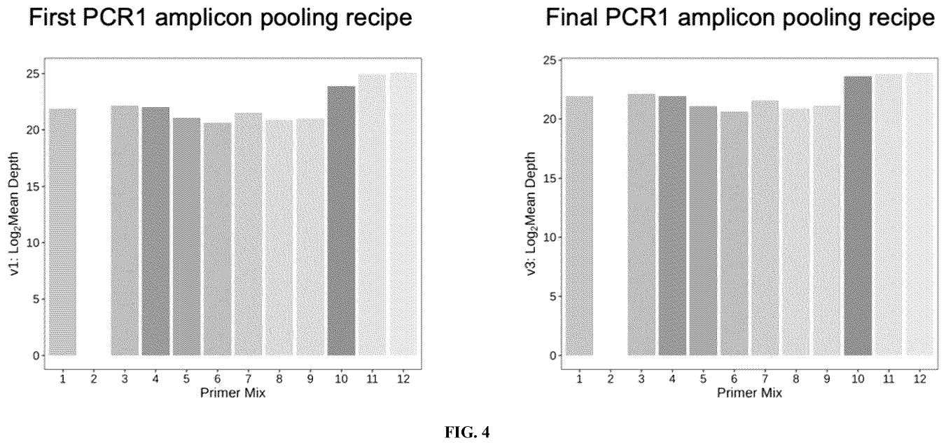

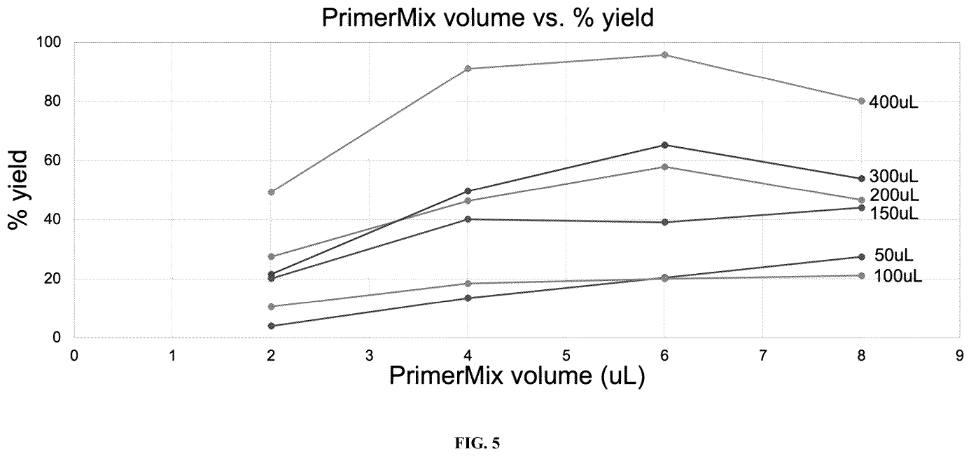

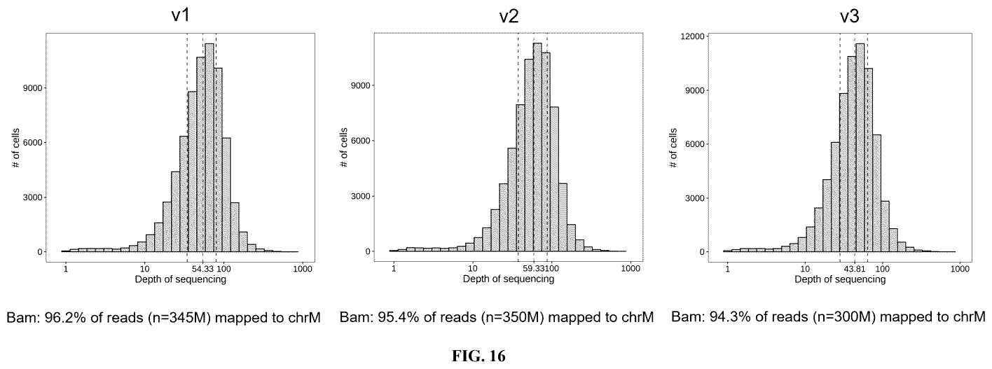

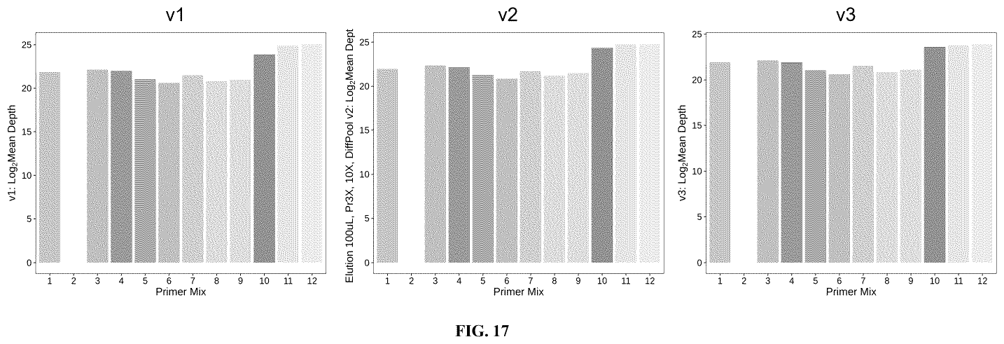

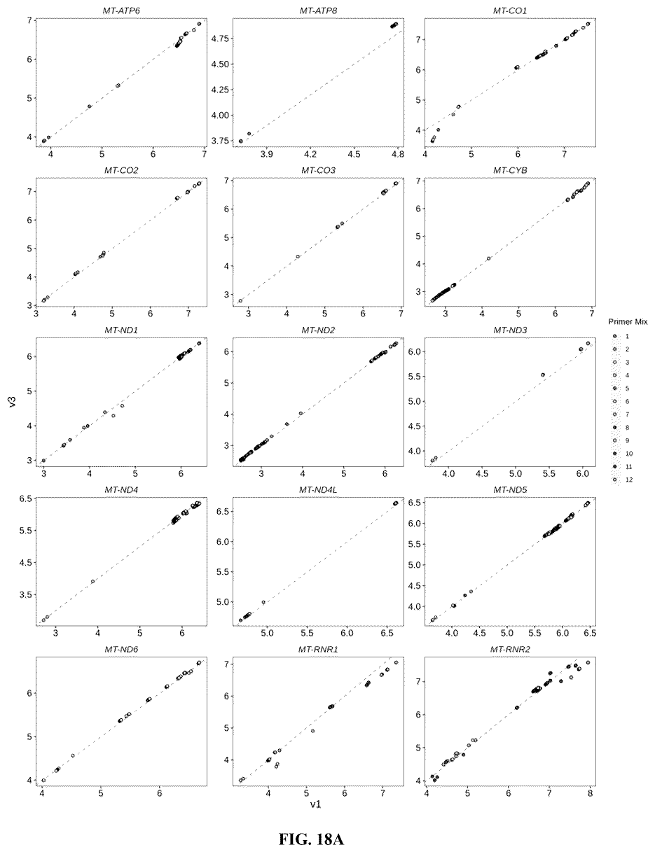

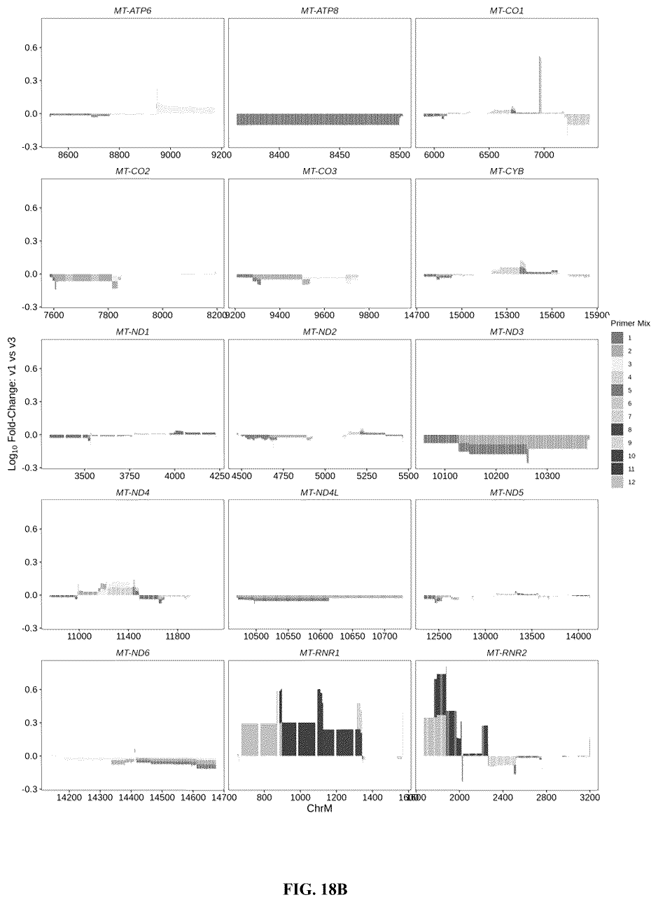

Experiments were undertaken to design a method for pooling the RNA sequencing primers (RSPs) and for pooling amplicon mixtures obtained from the 1^{st} PCR reaction of the method depicted in FIGS. 11A-11E using said pooled RSPs for use as templates for the 2^{nd} PCR reaction. The RSPs were pooled with each other to prepare 12 primer mixes (RSP_mixes), as described in Table 2. The primers were pooled with one another so that primers resulting in similar amplicon lengths were pooled with one another and so that two primers targeting the same gene were not included in the same primer mix. The 12 primer mixes were used to prepare 12 amplicon mixtures according to the method depicted in FIG. 11C. Experiments were undertaken to evaluate different strategies for the pooling of these 12 amplicon mixtures for use as templates for the 2^{nd} PCR reaction (FIG. 11D). The pooled 12 amplicon mixtures were cleaned using solid-phase reversible immobilization (SPRI) beads prior to being used as templates for the 2^{nd} PCR reaction. By pooling the amplicon mixtures by using 40 μL of each of the amplicon mixtures corresponding to primer mixes 1-9, 12 μL of the amplicon mixture corresponding to primer mix 10, and 8 μL of each of the amplicon mixtures corresponding to primer mixes 11 and 12, the overrepresentation of amplicons corresponding to RSP mixes 11 and 12 in the libraries prepared according to the method depicted in FIGS. 11A-11E was reduced by about 2-fold compared to the overrepresentation of amplicons corresponding to RSP mixes 11 and 12 resulting from pooling the amplicon mixtures by using 40 μL of each of the amplicon mixtures corresponding to primer mixes 1-9, 35 μL of the amplicon mixture corresponding to primer mix 10, and 32 μL of each of the amplicon mixtures corresponding to primer mixes 11 and 12 (FIGS. 4, 17, 18A, and 18B). The different pooling strategies evaluated were associated with only small changes in the percent of total reads mapping to the mitochondrial genome and/or depth of sequencing (FIG. 16).

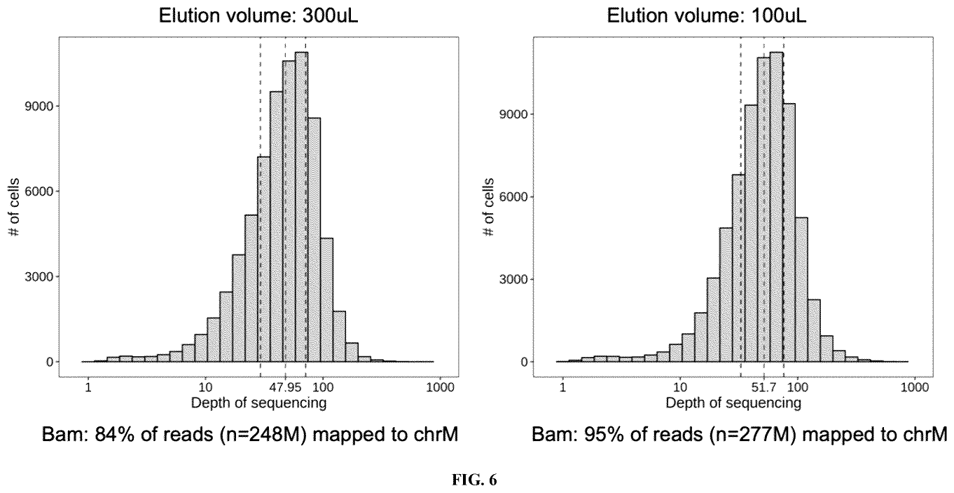

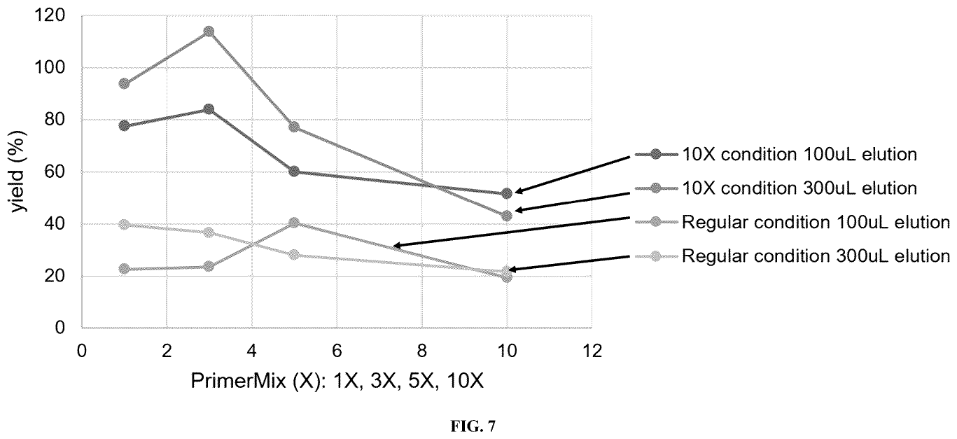

Experiments were undertaken to evaluate the impact of the volume of nuclease-free water (i.e., “elution volume”) used to elute the pooled amplicon mixtures from the SPRI beads used to clean the pooled amplicon mixtures prior to being used as templates for the 2^{nd} PCR reaction, which is depicted in FIG. 11D. When an elution volume of 20 μL was used, little to no amount of the resulting sequencing library was sequenceable, where sequenceability was measured using a KAPA Library Quantification kit. Assessing sequenceability of a library using a KAPA Library Quantification kit involves a qPCR assay using P5 and P7 primers. Without intending to be bound by theory, a small elution volume, such as 20 μL, may inhibit the 2^{nd} PCR reaction (i.e., the indexing PCR reaction) by overconcentrating the input amount of template for the 2^{nd} PCR reaction. An experiment was undertaken to assess the influence of elution volume and indexing PCR primer concentrations (see Table 3) on sequenceability of libraries prepared according to the method depicted in FIGS. 11A-11E (FIG. 5). 12 μL from each elution were used for the 2^{nd} PCR reaction. Significant PCR inhibition was observed with lower elution volumes (FIG. 5). An elution volume of 100 μL was selected as an elution volume for use in preparation of sequencing libraries going forward. The 100 μL elution volume resulted in a higher proportion of sequence reads mapping to the mitochondrial transcriptome (chrM) (95%) compared to an elution volume of 300 μL (84%) (FIG. 6), but the elution volumes of 100 μL and 300 μL were both associated with similar sequencing distribution of mitochondrial cDNA.

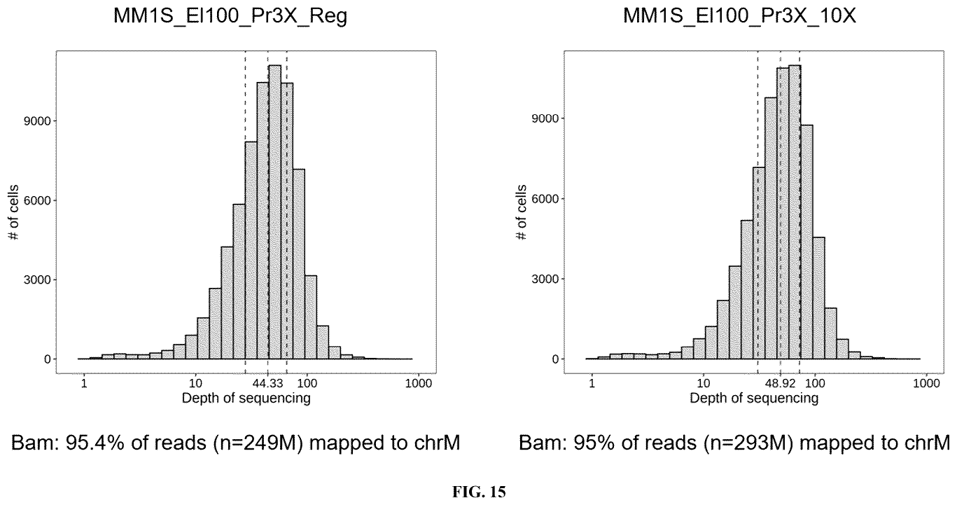

Experiments were then undertaken to evaluate different thermal cycling conditions and primer concentrations and ratios for the 2^{nd} PCR reaction, which is depicted in FIG. 11D. The sample indexing primers used in the 2nd PCR reaction are listed in Table 3. The concentrations of the forward and reverse primers were changed from 0.25 μM for each primer to 2.25 μM for the reverse primer and 0.45 μM for the forward primer. The forward to reverse primer ratio (F:R) used in the 2^{nd} PCR reaction was 1:5, the annealing temperature for the 2^{nd} PCR reaction was decreased from 60° C. to 54° C., and the initial denaturation time was increased from 30 s to 45 s. These modifications significantly improved the sequenceability of the libraries prepared according to the method depicted in FIGS. 11A-11E at elution volumes ranging from 50 μL to 300 μL, as assessed using a KAPA Library Quantification kit (FIG. 7). The decrease in annealing temperature was not associated with any decrease in amplification specificity (see FIG. 15).

### Example 2: Use of Targeted 5′-End Single-Cell RNA Sequencing of Mitochondrial RNA to Characterize Tumor Cells Obtained From Multiple Myeloma Patients

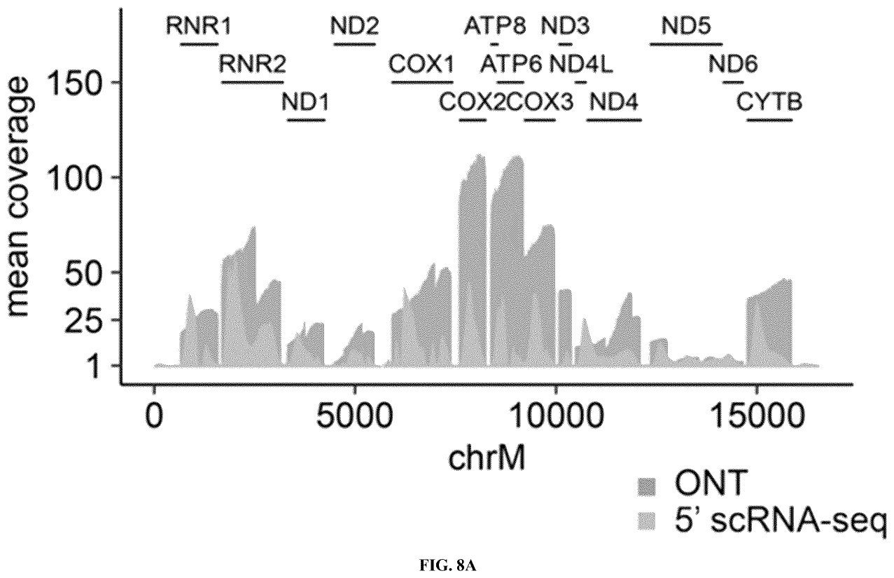

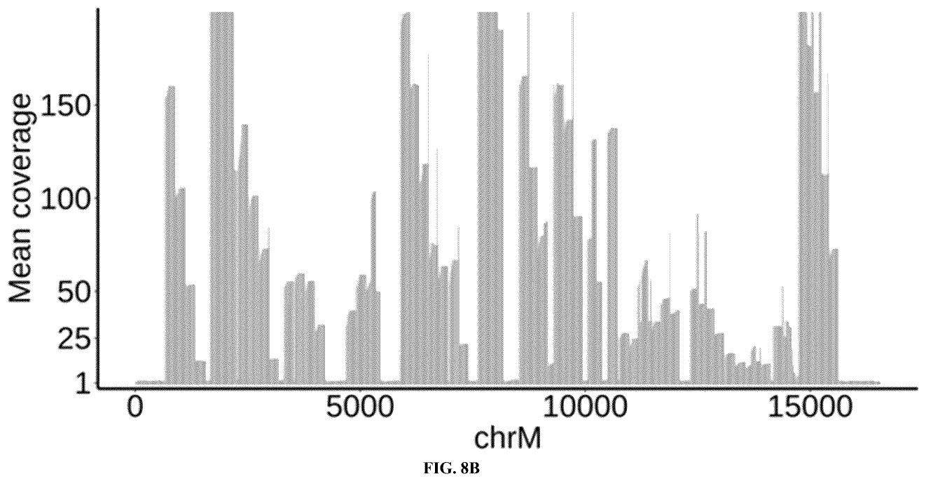

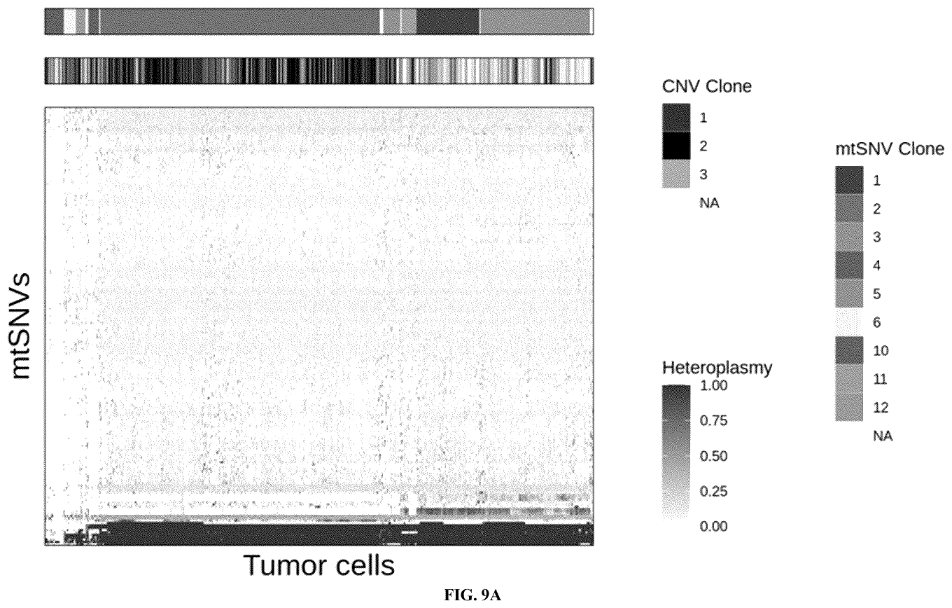

Experiments were undertaken to demonstrate the use of the method developed in Example 1 for 5′-end single-cell RNA sequencing of mitochondrial RNA (see FIG. 12) in the characterization of tumor cells from multiple myeloma patients. The method was used to perform single-cell mitochondrial transcript sequencing (scMito-seq) on four bone marrow samples from patients with Multiple Myeloma. Sequencing of the sequence libraries prepared according to the method provided good coverage of the mitochondrial transcriptome (mean: 81.62 coverage) (FIG. 8B). There was a notable improvement in coverage compared to that achieved using the method described by Penter L, et al. Nat Commun 15:32 (2024) (FIG. 8A), which used long-read sequencing, as compared to the short-read sequencing protocols of Example 1, 20 primers, a PCR to deplete artifacts, and an additional PCR step for some mitochondrial genes. Compared to long-read sequencing, short-read sequencing results in higher coverage at lower cost.

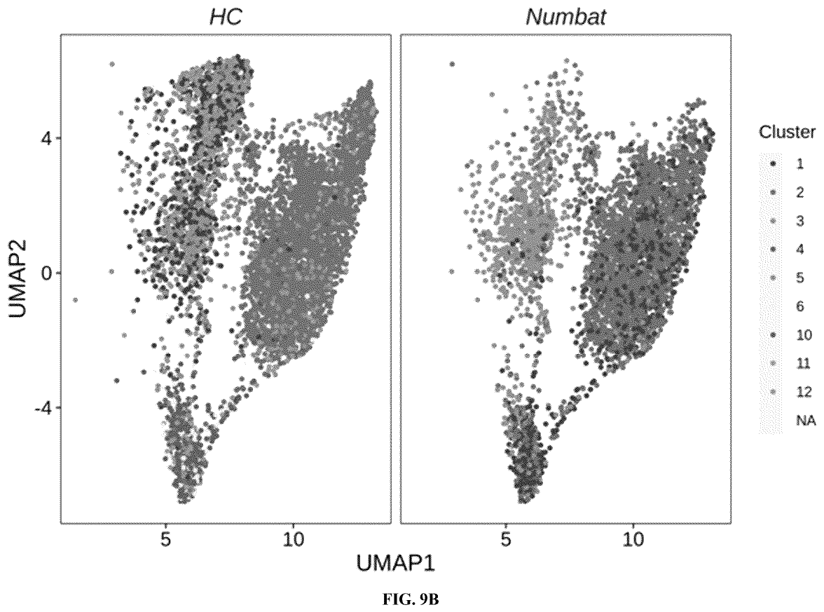

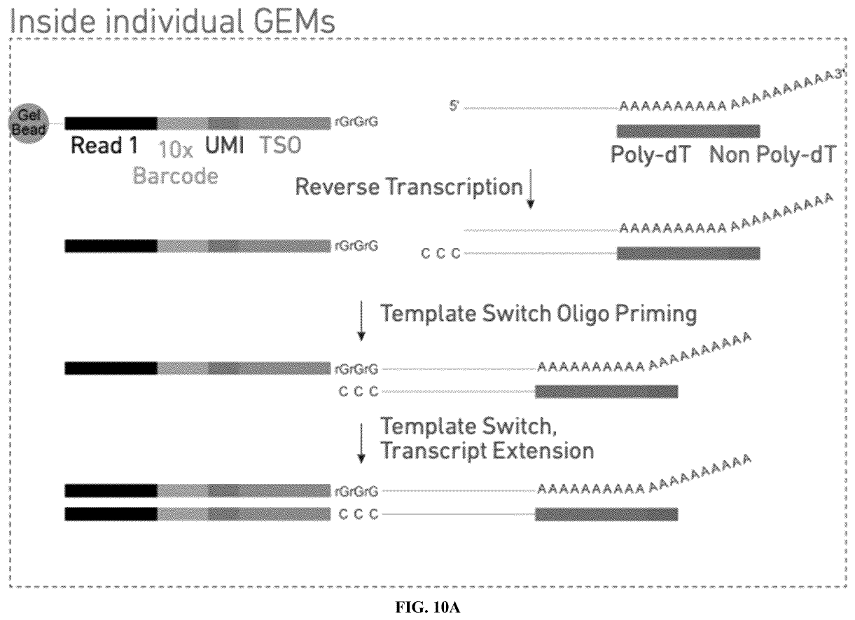

An experiment was next undertaken to demonstrate that mitochondrial mutations could be used to identify subclones of tumor cells within larger tumor populations (FIGS. 9A and 9B). For a patient known to have Multiple Myeloma with clonal amplification of chr1q and subclonal deletion of chr13q, sequencing of libraries prepared according to the single-cell mitochondrial RNA sequencing method of Example 1 led to excellent concordance of subclone identification, which was achieved through inferring copy number variants (CNVs) from the sequence data. The single-cell mitochondrial RNA sequencing data gave greater resolution and accuracy for subclone determination through the analysis of mitochondrial single nucleotide variants (SNVs) than could be achieved through CNV-based clone assignments, which tend to be noise-ridden, as can be seen through the identification of more subclones in the patient than previously-identified using CNV-based clone assignments (FIGS. 9A and 9B). Without intending to be bound by theory, this improved capacity for clone assignment may be because mitochondrial SNV-based clustering did not require the presence of a copy number variant (CNV), and thus could detect mutation-driven subclones. The 5′-end single-cell mitochondrial RNA sequencing method likely enables single-cell studies on the transcriptomic impact of mutations. These results demonstrate the superior capacity of the 5′-end single-cell mitochondrial RNA sequencing method to identify clonal and subclonal cell populations, be they CNV, structural variant, or mutation-driven, which may find application in research applications and/or clinical diagnostics.

### Sequences

Sequences for primers used in the above Example are provided in the following tables.

TABLE 1
Read 1 primer sequence (see 1st PCR portion of
FIGs. 11A -11E).

| Name | Nucleotide Sequence |
|---|---|
| R1 | ACACTCTTTCCCTACACGACGCTCTTCCGATCT |
| | (SEQ ID NO: 2) |

TABLE 2
RNA sequencing primer (RSP) sequences (see 1st PCR portions of FIGs. 10A-10E
and FIGs. 11A -11E).

| | | | | | | Start | Start | |
|---|---|---|---|---|---|---|---|---|
| | | | | | | position | position | |
| | SEQ | | | | | for | of | End |
| Primer | ID | | | GC | Length | amplifi- | target | of |
| Name | NO: | Targeting Sequence | Tₘ | % | (nt) | cation¹ | gene¹ | Gene |
| RSP_MT- | 16 | GGAGCGTTATGGAGTG | 64 | 55 | 20 | 9335 | 9207 | 9990 |
| CO3_4 | | GAAG | | | | | | |
| RSP_MT- | 17 | TGGTTCTCAGGGTTTG | 59 | 38.1 | 21 | 8523 | 8365 | 8526 |
| ATP8_1 | | TTATA | | | | | | |
| RSP_MT- | 18 | TAAGAGGGAGTGGGTG | 63 | 52.4 | 21 | 10634 | 10470 | 10759 |
| ND4L_2 | | TTGAG | | | | | | |
| RSP_MT- | 19 | GAGTGATGTGGGCGAT | 65 | 50 | 20 | 14957 | 14747 | 15887 |
| CYB_6 | | TGAT | | | | | | |
| RSP_MT- | 20 | GTTTGGGTTGTGGCTC | 66 | 55 | 20 | 12554 | 12337 | 14148 |
| ND5_9 | | AGTG | | | | | | |
| RSP_MT- | 21 | GTAGGGGTAAAAGGAG | 65 | 52.4 | 21 | 10284 | 10059 | 10404 |
| ND3_2 | | GGCAA | | | | | | |
| RSP_MT- | 22 | GATACTCCTCAATAGC | 61 | 47.6 | 21 | 14439 | 14673 | 14149 |
| ND6_3 | | CATCG | | | | | | |
| RSP_MT- | 23 | AGTTGCCAAAGCCTCC | 69 | 47.8 | 23 | 6144 | 5904 | 7445 |
| CO1_7 | | GATTATG | | | | | | |
| RSP_MT- | 24 | GAAGAGCGATGGTGAG | 64 | 55 | 20 | 3556 | 3306 | 4260 |
| ND1_4 | | AGCT | | | | | | |
| RSP_MT- | 25 | GATAAGTGGCGTTGGC | 67 | 55 | 20 | 11013 | 10759 | 12137 |
| ND4_7 | | TTGC | | | | | | |
| RSP_MT- | 26 | AGTCCGAGGAGGTTAG | 63 | 52.4 | 20 | 8786 | 8527 | 9207 |
| ATP6_3 | | TTGTG | | | | | | |
| RSP_MT- | 27 | TTGGTTATGGTTCATT | 60 | 40 | 20 | 4732 | 4470 | 5511 |
| ND2_5 | | GTCC | | | | | | |
| RSP_MT- | 28 | AGGTTTAGGTTATGTA | 56 | 37.5 | 24 | 10752 | 10470 | 10759 |
| ND4L_1 | | CGTAGTCT | | | | | | |
| RSP_MT- | 29 | GTAAGGGAGGGATCGT | 63 | 55 | 20 | 7872 | 7585 | 8269 |
| CO2_3 | | TGAC | | | | | | |
| RSP_MT- | 30 | CACCCACAGCACCAAT | 66 | 55 | 20 | 14356 | 14673 | 14149 |
| ND6_2 | | CCTA | | | | | | |
| RSP_MT- | 31 | TTCGGTTCAGTCTAAT | 58 | 40 | 20 | 10403 | 10059 | 10404 |
| ND3_1 | | CCTT | | | | | | |
| RSP_MT- | 32 | GCCAGTGCCCTCCTAA | 65 | 57.9 | 19 | 9554 | 9207 | 9990 |
| CO3_3 | | TTG | | | | | | |
| RSP_MT- | 33 | AGCAGGAGGATAATGC | 65 | 50 | 20 | 15108 | 14747 | 15887 |
| CYB_5 | | CGAT | | | | | | |
| RSP_MT- | 34 | GCGGTAACTAAGATTA | 58 | 37.5 | 24 | 12736 | 12337 | 14148 |
| ND5_8 | | GTATGGTA | | | | | | |
| RSP_MT- | 35 | GATGGTTAGGTCTACG | 61 | 55 | 20 | 6344 | 5904 | 7445 |
| CO1_6 | | GAGG | | | | | | |
| RSP_MT- | 36 | GCTGATGGTTTCGATA | 60 | 37.5 | 24 | 8970 | 8527 | 9207 |
| ATP6_2 | | ATAACTAG | | | | | | |
| RSP_MT- | 37 | TTGAGAGAGTGAGGAG | 63 | 52.4 | 21 | 4944 | 4470 | 5511 |
| ND2_4 | | AAGGC | | | | | | |
| RSP_MT- | 38 | GAGTAGGGGAAGGGAG | 64 | 60 | 20 | 11242 | 10759 | 12137 |
| ND4_6 | | CCTA | | | | | | |
| RSP_MT- | 39 | GTGGAGAGGTTAAAGG | 65 | 54.5 | 22 | 3797 | 3306 | 4260 |
| ND1_3 | | AGCCAC | | | | | | |
| RSP_MT- | 40 | CCTAATGTGGGGACAG | 65 | 52.4 | 21 | 8091 | 7585 | 8269 |
| CO2_2 | | CTCAT | | | | | | |
| RSP_MT- | 41 | CGAGCAATCTCAATTA | 58 | 31.8 | 22 | 14159 | 14673 | 14149 |
| ND6_1 | | CAATAT | | | | | | |
| RSP_MT- | 42 | AATCGTGTGAGGGTGG | 66 | 55 | 20 | 15279 | 14747 | 15887 |
| CYB_4 | | GACT | | | | | | |
| RSP_MT- | 43 | GTCGGAAATGGTGAAG | 66 | 55 | 20 | 9776 | 9207 | 9990 |
| CO3_2 | | GGAG | | | | | | |
| RSP_MT- | 44 | GGATAAATCATGCTAA | 63 | 42.9 | 21 | 12907 | 12337 | 14148 |
| ND5_7 | | GGCGA | | | | | | |
| RSP_MT- | 45 | GGTCGAAGAAGGTGGT | 65 | 55 | 20 | 6567 | 5904 | 7445 |
| CO1_5 | | GTTG | | | | | | |
| RSP_MT- | 46 | GGCTTCGACATGGGCT | 66 | 55 | 20 | 11428 | 10759 | 12137 |
| ND4_5 | | TTAG | | | | | | |
| RSP_MT- | 47 | TGTCGTGCAGGTAGAG | 66 | 54.5 | 22 | 9199 | 8527 | 9207 |
| ATP6_1 | | GCTTAC | | | | | | |
| RSP_MT- | 48 | TGCGAGATAGTAGTAG | 61 | 52.4 | 21 | 5166 | 4470 | 5511 |
| ND2_3 | | GGTCG | | | | | | |
| RSP_MT- | 49 | GACGATGGGCATGAAA | 65 | 50 | 20 | 8215 | 7585 | 8269 |
| CO2_1 | | CTGT | | | | | | |
| RSP_MT- | 50 | GGAAGAGAAGTAAGCC | 62 | 55 | 20 | 15452 | 14747 | 15887 |
| CYB_3 | | GAGG | | | | | | |
| RSP_MT- | 51 | AGATTGTAGTGGTGAG | 62 | 45.5 | 22 | 4030 | 3306 | 4260 |
| ND1_2 | | GGTGTT | | | | | | |
| RSP_MT- | 52 | TTGAGAATGAGTGTGA | 65 | 47.6 | 21 | 11511 | 10759 | 12137 |
| ND4_4 | | GGCGT | | | | | | |
| RSP_MT- | 53 | GATGGAGACATACAGA | 56 | 39.1 | 23 | 9974 | 9207 | 9990 |
| CO3_1 | | AATAGTC | | | | | | |
| RSP_MT- | 54 | GTGGAAGCGGATGAGT | 64 | 47.6 | 21 | 13128 | 12337 | 14148 |
| ND5_6 | | AAGAA | | | | | | |
| RSP_MT- | 55 | TCACACGATAAACCCT | 65 | 47.8 | 23 | 6764 | 5904 | 7445 |
| CO1_4 | | AGGAAGC | | | | | | |
| RSP_MT- | 56 | GGAGTAGTGTGATTGA | 64 | 52.2 | 23 | 5385 | 4470 | 5511 |
| ND2_2 | | GGTGGAG | | | | | | |
| RSP_MT- | 57 | ATGGGGATTATTGCTA | 62 | 42.9 | 21 | 15663 | 14747 | 15887 |
| CYB_2 | | GGATG | | | | | | |
| RSP_MT- | 58 | GGGAATGCTGGAGATT | 65 | 45.5 | 22 | 4250 | 3306 | 4260 |
| ND1_1 | | GTAATG | | | | | | |
| RSP_MT- | 59 | GCGATTATGAGAATGA | 60 | 45 | 20 | 11712 | 10759 | 12137 |
| ND4_ 3 | | CTGC | | | | | | |
| RSP_MT- | 60 | AGATAGGTAGGAGTAG | 59 | 52.4 | 21 | 5487 | 4470 | 5511 |
| ND2_1 | | CGTGG | | | | | | |
| RSP_MT- | 61 | TAATACAATGCCAGTC | 61 | 45 | 20 | 6976 | 5904 | 7445 |
| CO1_3 | | AGGC | | | | | | |
| RSP_MT- | 62 | GACCTGTGGGTTTGTT | 62 | 47.8 | 23 | 2779 | 1671 | 3229 |
| RNR2_3 | | AGGTACT | | | | | | |
| RSP_MT- | 63 | TTGTCAGGGAGGTAGC | 66 | 55 | 20 | 13594 | 12337 | 14148 |
| ND5_4 | | GATG | | | | | | |
| RSP_MT- | 64 | CTCCCTAATTGAAAAC | 58 | 30.4 | 23 | 15875 | 14747 | 15887 |
| CYB_1 | | AAAATAC | | | | | | |
| RSP_MT- | 65 | GATCAGGAGAACGTGG | 60 | 45.5 | 22 | 11925 | 10759 | 12137 |
| ND4_2 | | TTACTA | | | | | | |
| RSP_MT- | 66 | GGTAGTCCGAGTAACG | 64 | 60 | 20 | 7233 | 5904 | 7445 |
| CO1_2 | | TCGG | | | | | | |
| RSP_MT- | 67 | CTAGGAAAGTGACAGC | 62 | 55 | 20 | 13825 | 12337 | 14148 |
| ND5_3 | | GAGG | | | | | | |
| RSP_MT- | 68 | ATCGGGATGTCCTGAT | 68 | 52.4 | 21 | 3012 | 1671 | 3229 |
| RNR2_2 | | CCAAC | | | | | | |
| RSP_MT- | 69 | GAAAACCCGGTAATGA | 68 | 50 | 22 | 12132 | 10759 | 12137 |
| ND4_1 | | TGTCGG | | | | | | |
| RSP_MT- | 70 | TTATGTATACGGGTTC | 57 | 40 | 20 | 7437 | 5904 | 7445 |
| CO1_1 | | TTCG | | | | | | |
| RSP_MT- | 71 | TGTGCGGTGTGTGATG | 67 | 55 | 20 | 13944 | 12337 | 14148 |
| ND5_2 | | CTAG | | | | | | |
| RSP_MT- | 72 | GTTCTTGGGTGGGTGT | 68 | 54.4 | 22 | 3222 | 1671 | 3229 |
| RNR2_1 | | GGGTAT | | | | | | |
| RSP_MT- | 73 | GTGATTAGGAGTAGGG | 56 | 47.6 | 21 | 14144 | 12337 | 14148 |
| ND5_1 | | TTAGG | | | | | | |
| RSP_MT- | 74 | GCGGTGCCTCTAATAC | 63 | 55 | 20 | 2543 | 1671 | 3229 |
| RNR2_4 | | TGGT | | | | | | |
| RSP_MT- | 75 | GCACTTTCCAGTACAC | 58 | 47.6 | 21 | 1589 | 647 | 1601 |
| RNR1_1 | | TTACC | | | | | | |
| RSP_MT- | 76 | CGGAGCACATAAATAG | 63 | 45.5 | 22 | 13366 | 12337 | 14148 |
| ND5_5 | | TATGGC | | | | | | |
| RSP_MT- | 77 | ATAGGGTGATAGATTG | 57 | 45 | 20 | 2287 | 1671 | 3229 |
| RNR2_5 | | GTCC | | | | | | |
| RSP_MT- | 78 | TGTAGCCCATTTCTTG | 65 | 50 | 20 | 1367 | 647 | 1601 |
| RNR1_2 | | CCAC | | | | | | |
| RSP_MT- | 79 | GTTGAACTAAGATTCT | 61 | 36 | 25 | 2046 | 1671 | 3229 |
| RNR2_6 | | ATCTTGGGA | | | | | | |
| RSP_MT- | 80 | GTAGTGTTCTGGCGAG | 63 | 55 | 20 | 1146 | 647 | 1601 |
| RNR1_3 | | CAGT | | | | | | |
| RSP_MT- | 81 | CTTAGCTTTGGCTCTC | 65 | 52.4 | 21 | 1899 | 1671 | 3229 |
| RNR2_7 | | CTTGC | | | | | | |
| RSP_MT- | 82 | TGGGTTAATCGTGTGA | 68 | 55 | 20 | 918 | 647 | 1601 |
| RNR1_4 | | CCGC | | | | | | |

| | | | | | Identification | |
|---|---|---|---|---|---|---|
| | | | | | Number for | |
| | | | | | Corresponding | |
| | | | | | RNA | Length |
| | | | | | Sequencing | of |
| | SEQ | | SEQ | Full-length RNA | Primer | Amplified |
| Primer | ID | Read 2 | ID | Sequencing Primer | Mixture | cDNA |
| Name | NO: | Sequence | NO: | (RSP) Sequence | (RSP_Mix) | Fragment |
| RSP_MT- | 3 | CACCCGAGAA | 83 | CACCCGAGAATTCCAGGAGCGTT | 1 | 128 |
| CO3 4 | | TTCCA | | ATGGAGTGGAAG | | |
| RSP_MT- | 3 | CACCCGAGAA | 84 | CACCCGAGAATTCCATGGTTCTC | 1 | 158 |
| ATP8 1 | | TTCCA | | AGGGTTTGTTATA | | |
| RSP_MT- | 3 | CACCCGAGAA | 85 | CACCCGAGAATTCCATAAGAGGG | 1 | 164 |
| ND4L 2 | | TTCCA | | AGTGGGTGTTGAG | | |
| RSP_MT- | 3 | CACCCGAGAA | 86 | CACCCGAGAATTCCAGAGTGATG | 1 | 210 |
| CYB_6 | | TTCCA | | TGGGCGATTGAT | | |
| RSP_MT- | 3 | CACCCGAGAA | 87 | CACCCGAGAATTCCAGTTTGGGT | 1 | 217 |
| ND5_9 | | TTCCA | | TGTGGCTCAGTG | | |
| RSP_MT- | 3 | CACCCGAGAA | 88 | CACCCGAGAATTCCAGTAGGGGT | 1 | 225 |
| ND3 2 | | TTCCA | | AAAAGGAGGGCAA | | |
| RSP_MT- | 3 | CACCCGAGAA | 89 | CACCCGAGAATTCCAGATACTCC | 1 | 234 |
| ND6 3 | | TTCCA | | TCAATAGCCATCG | | |
| RSP_MT- | 3 | CACCCGAGAA | 90 | CACCCGAGAATTCCAAGTTGCCA | 1 | 240 |
| CO1_7 | | TTCCA | | AAGCCTCCGATTATG | | |
| RSP_MT- | 3 | CACCCGAGAA | 91 | CACCCGAGAATTCCAGAAGAGCG | 1 | 250 |
| ND1 4 | | TTCCA | | ATGGTGAGAGCT | | |
| RSP_MT- | 3 | CACCCGAGAA | 92 | CACCCGAGAATTCCAGATAAGTG | 1 | 254 |
| ND4 7 | | TTCCA | | GCGTTGGCTTGC | | |
| RSP_MT- | 3 | CACCCGAGAA | 93 | CACCCGAGAATTCCAAGTCCGAG | 1 | 259 |
| ATP6 3 | | TTCCA | | GAGGTTAGTTGTG | | |
| RSP_MT- | 3 | CACCCGAGAA | 94 | CACCCGAGAATTCCATTGGTTAT | 1 | 262 |
| ND2 5 | | TTCCA | | GGTTCATTGTCC | | |
| RSP_MT- | 3 | CACCCGAGAA | 95 | CACCCGAGAATTCCAAGGTTTAG | 2 | 282 |
| ND4L 1 | | TTCCA | | GTTATGTACGTAGTCT | | |
| RSP_MT- | 3 | CACCCGAGAA | 96 | CACCCGAGAATTCCAGTAAGGGA | 2 | 287 |
| CO2 3 | | TTCCA | | GGGATCGTTGAC | | |
| RSP_MT- | 3 | CACCCGAGAA | 97 | CACCCGAGAATTCCACACCCACA | 2 | 317 |
| ND6 2 | | TTCCA | | GCACCAATCCTA | | |
| RSP_MT- | 3 | CACCCGAGAA | 98 | CACCCGAGAATTCCATTCGGTTC | 2 | 344 |
| ND3 1 | | TTCCA | | AGTCTAATCCTT | | |
| RSP_MT- | 3 | CACCCGAGAA | 99 | CACCCGAGAATTCCAGCCAGTGC | 2 | 347 |
| CO3 3 | | TTCCA | | CCTCCTAATTG | | |
| RSP_MT- | 3 | CACCCGAGAA | 100 | CACCCGAGAATTCCAAGCAGGAG | 2 | 361 |
| CYB 5 | | TTCCA | | GATAATGCCGAT | | |
| RSP_MT- | 3 | CACCCGAGAA | 101 | CACCCGAGAATTCCAGCGGTAAC | 2 | 399 |
| ND5 8 | | TTCCA | | TAAGATTAGTATGGTA | | |
| RSP_MT- | 3 | CACCCGAGAA | 102 | CACCCGAGAATTCCAGATGGTTA | 2 | 440 |
| CO1 6 | | TTCCA | | GGTCTACGGAGG | | |
| RSP_MT- | 3 | CACCCGAGAA | 103 | CACCCGAGAATTCCAGCTGATGG | 2 | 443 |
| ATP6_2 | | TTCCA | | TTTCGATAATAACTAG | | |
| RSP_MT- | 3 | CACCCGAGAA | 104 | CACCCGAGAATTCCATTGAGAGA | 2 | 474 |
| ND2 4 | | TTCCA | | GTGAGGAGAAGGC | | |
| RSP_MT- | 3 | CACCCGAGAA | 105 | CACCCGAGAATTCCAGAGTAGGG | 2 | 483 |
| ND4 6 | | TTCCA | | GAAGGGAGCCTA | | |
| RSP_MT- | 3 | CACCCGAGAA | 106 | CACCCGAGAATTCCAGTGGAGAG | 2 | 491 |
| ND1 3 | | TTCCA | | GTTAAAGGAGCCAC | | |
| RSP_MT- | 3 | CACCCGAGAA | 107 | CACCCGAGAATTCCACCTAATGT | 3 | 506 |
| CO2 2 | | TTCCA | | GGGGACAGCTCAT | | |
| RSP_MT- | 3 | CACCCGAGAA | 108 | CACCCGAGAATTCCACGAGCAAT | 3 | 514 |
| ND6 1 | | TTCCA | | CTCAATTACAATAT | | |
| RSP_MT- | 3 | CACCCGAGAA | 109 | CACCCGAGAATTCCAAATCGTGT | 3 | 532 |
| CYB 4 | | TTCCA | | GAGGGTGGGACT | | |
| RSP_MT- | 3 | CACCCGAGAA | 110 | CACCCGAGAATTCCAGTCGGAAA | 3 | 569 |
| CO3 2 | | TTCCA | | TGGTGAAGGGAG | | |
| RSP_MT- | 3 | CACCCGAGAA | 111 | CACCCGAGAATTCCAGGATAAAT | 3 | 570 |
| ND5 7 | | TTCCA | | CATGCTAAGGCGA | | |
| RSP_MT- | 3 | CACCCGAGAA | 112 | CACCCGAGAATTCCAGGTCGAAG | 3 | 663 |
| CO1 5 | | TTCCA | | AAGGTGGTGTTG | | |
| RSP_MT- | 3 | CACCCGAGAA | 113 | CACCCGAGAATTCCAGGCTTCGA | 3 | 669 |
| ND4 5 | | TTCCA | | CATGGGCTTTAG | | |
| RSP_MT- | 3 | CACCCGAGAA | 114 | CACCCGAGAATTCCATGTCGTGC | 3 | 672 |
| ATP6 1 | | TTCCA | | AGGTAGAGGCTTAC | | |
| RSP_MT- | 3 | CACCCGAGAA | 115 | CACCCGAGAATTCCATGCGAGAT | 3 | 696 |
| ND2 3 | | TTCCA | | AGTAGTAGGGTCG | | |
| RSP_MT- | 3 | CACCCGAGAA | 116 | CACCCGAGAATTCCAGACGATGG | 4 | 630 |
| CO2 1 | | TTCCA | | GCATGAAACTGT | | |
| RSP_MT- | 3 | CACCCGAGAA | 117 | CACCCGAGAATTCCAGGAAGAGA | 4 | 705 |
| CYB 3 | | TTCCA | | AGTAAGCCGAGG | | |
| RSP_MT- | 3 | CACCCGAGAA | 118 | CACCCGAGAATTCCAAGATTGTA | 4 | 724 |
| ND1 2 | | TTCCA | | GTGGTGAGGGTGTT | | |
| RSP_MT- | 3 | CACCCGAGAA | 119 | CACCCGAGAATTCCATTGAGAAT | 4 | 752 |
| ND4 4 | | TTCCA | | GAGTGTGAGGCGT | | |
| RSP_MT- | 3 | CACCCGAGAA | 120 | CACCCGAGAATTCCAGATGGAGA | 4 | 767 |
| CO3 1 | | TTCCA | | CATACAGAAATAGTC | | |
| RSP_MT- | 3 | CACCCGAGAA | 121 | CACCCGAGAATTCCAGTGGAAGC | 4 | 791 |
| ND5 6 | | TTCCA | | GGATGAGTAAGAA | | |
| RSP_MT- | 3 | CACCCGAGAA | 122 | CACCCGAGAATTCCATCACACGA | 4 | 860 |
| CO1 4 | | TTCCA | | TAAACCCTAGGAAGC | | |
| RSP_MT- | 3 | CACCCGAGAA | 123 | CACCCGAGAATTCCAGGAGTAGT | 4 | 915 |
| ND2 2 | | TTCCA | | GTGATTGAGGTGGAG | | |
| RSP_MT- | 3 | CACCCGAGAA | 124 | CACCCGAGAATTCCAATGGGGAT | 5 | 916 |
| CYB 2 | | TTCCA | | TATTGCTAGGATG | | |
| RSP_MT- | 3 | CACCCGAGAA | 125 | CACCCGAGAATTCCAGGGAATGC | 5 | 944 |
| ND1 1 | | TTCCA | | TGGAGATTGTAATG | | |
| RSP_MT- | 3 | CACCCGAGAA | 126 | CACCCGAGAATTCCAGCGATTAT | 5 | 953 |
| ND4 3 | | TTCCA | | GAGAATGACTGC | | |
| RSP_MT- | 3 | CACCCGAGAA | 127 | CACCCGAGAATTCCAAGATAGGT | 5 | 1017 |
| ND2 1 | | TTCCA | | AGGAGTAGCGTGG | | |
| RSP_MT- | 3 | CACCCGAGAA | 128 | CACCCGAGAATTCCATAATACAA | 5 | 1072 |
| CO1 3 | | TTCCA | | TGCCAGTCAGGC | | |
| RSP_MT- | 3 | CACCCGAGAA | 129 | CACCCGAGAATTCCAGACCTGTG | 5 | 1108 |
| RNR2 3 | | TTCCA | | GGTTTGTTAGGTACT | | |
| RSP_MT- | 3 | CACCCGAGAA | 130 | CACCCGAGAATTCCATTGTCAGG | 5 | 1257 |
| ND5 4 | | TTCCA | | GAGGTAGCGATG | | |
| RSP_MT- | 3 | CACCCGAGAA | 131 | CACCCGAGAATTCCACTCCCTAA | 6 | 1128 |
| CYB 1 | | TTCCA | | TTGAAAACAAAATAC | | |
| RSP_MT- | 3 | CACCCGAGAA | 132 | CACCCGAGAATTCCAGATCAGGA | 6 | 1166 |
| ND4 2 | | TTCCA | | GAACGTGGTTACTA | | |
| RSP_MT- | 3 | CACCCGAGAA | 133 | CACCCGAGAATTCCAGGTAGTCC | 6 | 1329 |
| CO1 2 | | TTCCA | | GAGTAACGTCGG | | |
| RSP_MT- | 3 | CACCCGAGAA | 134 | CACCCGAGAATTCCACTAGGAAA | 6 | 1488 |
| ND5 3 | | TTCCA | | GTGACAGCGAGG | | |
| RSP_MT- | 3 | CACCCGAGAA | 135 | CACCCGAGAATTCCAATCGGGAT | 7 | 1341 |
| RNR2 2 | | TTCCA | | GTCCTGATCCAAC | | |
| RSP_MT- | 3 | CACCCGAGAA | 136 | CACCCGAGAATTCCAGAAAACCC | 7 | 1373 |
| ND4 1 | | TTCCA | | GGTAATGATGTCGG | | |
| RSP_MT- | 3 | CACCCGAGAA | 137 | CACCCGAGAATTCCATTATGTAT | 7 | 1533 |
| CO1 1 | | TTCCA | | ACGGGTTCTTCG | | |
| RSP_MT- | 3 | CACCCGAGAA | 138 | CACCCGAGAATTCCATGTGCGGT | 7 | 1607 |
| ND5 2 | | TTCCA | | GTGTGATGCTAG | | |
| RSP_MT- | 3 | CACCCGAGAA | 139 | CACCCGAGAATTCCAGTTCTTGG | 8 | 1551 |
| RNR2 1 | | TTCCA | | GTGGGTGTGGGTAT | | |
| RSP_MT- | 3 | CACCCGAGAA | 140 | CACCCGAGAATTCCAGTGATTAG | 8 | 1807 |
| ND5 1 | | TTCCA | | GAGTAGGGTTAGG | | |
| RSP_MT- | 3 | CACCCGAGAA | 141 | CACCCGAGAATTCCAGCGGTGCC | 9 | 872 |
| RNR2 4 | | TTCCA | | TCTAATACTGGT | | |
| RSP_MT- | 3 | CACCCGAGAA | 142 | CACCCGAGAATTCCAGCACTTTC | 9 | 942 |
| RNR1 1 | | TTCCA | | CAGTACACTTACC | | |
| RSP_MT- | 3 | CACCCGAGAA | 143 | CACCCGAGAATTCCACGGAGCAC | 9 | 1029 |
| ND5 5 | | TTCCA | | ATAAATAGTATGGC | | |
| RSP_MT- | 3 | CACCCGAGAA | 144 | CACCCGAGAATTCCAATAGGGTG | 10 | 616 |
| RNR2 5 | | TTCCA | | ATAGATTGGTCC | | |
| RSP_MT- | 3 | CACCCGAGAA | 145 | CACCCGAGAATTCCATGTAGCCC | 10 | 720 |
| RNR1 2 | | TTCCA | | ATTTCTTGCCAC | | |
| RSP_MT- | 3 | CACCCGAGAA | 146 | CACCCGAGAATTCCAGTTGAACT | 11 | 375 |
| RNR2 6 | | TTCCA | | AAGATTCTATCTTGGGA | | |
| RSP_MT- | 3 | CACCCGAGAA | 147 | CACCCGAGAATTCCAGTAGTGTT | 11 | 499 |
| RNR1 3 | | TTCCA | | CTGGCGAGCAGT | | |
| RSP_MT- | 3 | CACCCGAGAA | 148 | CACCCGAGAATTCCACTTAGCTT | 12 | 228 |
| RNR2 7 | | TTCCA | | TGGCTCTCCTTGC | | |
| RSP_MT- | 3 | CACCCGAGAA | 149 | CACCCGAGAATTCCATGGGTTAA | 12 | 271 |
| RNR1 4 | | TTCCA | | TCGTGTGACCGC | | |

¹Positions are provided as nucleotide positions within the mitochondrial genome.

TABLE 3
Sample indexing PCR primer sequences (see 2nd PCR portion of FIGs. 11A -11E).

| | SEQ ID | |
|---|---|---|
| Primer Name | NO: | Sequence (5′ to 3′) |
| XV-P7-i7-BC01 | 150 | CAAGCAGAAGACGGCATACGAGATAAGTAGAGGTGACTGGAGTTCCTTGGCACC |
| | | CGAGAATTCCA |
| XV-P7-i7-BC02 | 151 | CAAGCAGAAGACGGCATACGAGATGGTCCAGAGTGACTGGAGTTCCTTGGCACC |
| | | CGAGAATTCCA |
| XV-P7-17-BC03 | 152 | CAAGCAGAAGACGGCATACGAGATGCACATCTGTGACTGGAGTTCCTTGGCACC |
| | | CGAGAATTCCA |
| XV-P7-17-BC04 | 153 | CAAGCAGAAGACGGCATACGAGATTTCGCTGAGTGACTGGAGTTCCTTGGCACC |
| | | CGAGAATTCCA |
| XV-P7-i7-BC05 | 154 | CAAGCAGAAGACGGCATACGAGATAGCAATTCGTGACTGGAGTTCCTTGGCACC |
| | | CGAGAATTCCA |
| XV-P7-i7-BC06 | 155 | CAAGCAGAAGACGGCATACGAGATCACATCCTGTGACTGGAGTTCCTTGGCACC |
| | | CGAGAATTCCA |
| XV-P7-i7-BC07 | 156 | CAAGCAGAAGACGGCATACGAGATCCAGTTAGGTGACTGGAGTTCCTTGGCACC |
| | | CGAGAATTCCA |
| XV-P7-i7-BC08 | 157 | CAAGCAGAAGACGGCATACGAGATAAGGATGTGTGACTGGAGTTCCTTGGCACC |
| | | CGAGAATTCCA |
| XV-P7-17-BC09 | 158 | CAAGCAGAAGACGGCATACGAGATACACGATCGTGACTGGAGTTCCTTGGCACC |
| | | CGAGAATTCCA |
| XV-P7-i7-BC10 | 159 | CAAGCAGAAGACGGCATACGAGATCATGCTTAGTGACTGGAGTTCCTTGGCACC |
| | | CGAGAATTCCA |
| XV-P7-i7-BC11 | 160 | CAAGCAGAAGACGGCATACGAGATGTATAACAGTGACTGGAGTTCCTTGGCACC |
| | | CGAGAATTCCA |
| XV-P7-i7-BC12 | 161 | CAAGCAGAAGACGGCATACGAGATTGCTCGACGTGACTGGAGTTCCTTGGCACC |
| | | CGAGAATTCCA |
| XV-P7-i7-BC13 | 162 | CAAGCAGAAGACGGCATACGAGATAACTTGACGTGACTGGAGTTCCTTGGCACC |
| | | CGAGAATTCCA |
| XV-P7-17-BC14 | 163 | CAAGCAGAAGACGGCATACGAGATAGTTGCTTGTGACTGGAGTTCCTTGGCACC |
| | | CGAGAATTCCA |
| XV-P7-i7-BC15 | 164 | CAAGCAGAAGACGGCATACGAGATTCGGAATGGTGACTGGAGTTCCTTGGCACC |
| | | CGAGAATTCCA |
| XV-P7-i7-BC16 | 165 | CAAGCAGAAGACGGCATACGAGATTTGAGCCTGTGACTGGAGTTCCTTGGCACC |
| | | CGAGAATTCCA |
| XV-P7-i7-BC17 | 166 | CAAGCAGAAGACGGCATACGAGATTGTTCCGAGTGACTGGAGTTCCTTGGCACC |
| | | CGAGAATTCCA |
| XV-P7-i7-BC18 | 167 | CAAGCAGAAGACGGCATACGAGATAGGATCTAGTGACTGGAGTTCCTTGGCACC |
| | | CGAGAATTCCA |
| XV-P7-i7-BC19 | 168 | CAAGCAGAAGACGGCATACGAGATCATAGCGAGTGACTGGAGTTCCTTGGCACC |
| | | CGAGAATTCCA |
| XV-P7-17-BC20 | 169 | CAAGCAGAAGACGGCATACGAGATCCTATGCCGTGACTGGAGTTCCTTGGCACC |
| | | CGAGAATTCCA |
| XV-P7-i7-BC21 | 170 | CAAGCAGAAGACGGCATACGAGATTGTCGGATGTGACTGGAGTTCCTTGGCACC |
| | | CGAGAATTCCA |
| XV-P7-i7-BC22 | 171 | CAAGCAGAAGACGGCATACGAGATATAGCGTCGTGACTGGAGTTCCTTGGCACC |
| | | CGAGAATTCCA |
| XV-P7-i7-BC23 | 172 | CAAGCAGAAGACGGCATACGAGATCCTACCATGTGACTGGAGTTCCTTGGCACC |
| | | CGAGAATTCCA |
| XV-P7-i7-BC24 | 173 | CAAGCAGAAGACGGCATACGAGATATTCTAGGGTGACTGGAGTTCCTTGGCACC |
| | | CGAGAATTCCA |
| XV-P5-15-BC01 | 174 | AATGATACGGCGACCACCGAGATCTACACATCGACTGACACTCTTTCC |
| XV-P5-15-BC02 | 175 | AATGATACGGCGACCACCGAGATCTACACCGGTTCTTACACTCTTTCC |
| XV-P5-15-BC03 | 176 | AATGATACGGCGACCACCGAGATCTACACAACCTCTTACACTCTTTCC |
| XV-P5-15-BC04 | 177 | AATGATACGGCGACCACCGAGATCTACACCGCATATTACACTCTTTCC |
| XV-P5-15-BC05 | 178 | AATGATACGGCGACCACCGAGATCTACACCTGCTCCTACACTCTTTCC |
| XV-P5-15-BC06 | 179 | AATGATACGGCGACCACCGAGATCTACACGCTGCACTACACTCTTTCC |
| XV-P5-15-BC07 | 180 | AATGATACGGCGACCACCGAGATCTACACCCTGTCATACACTCTTTCC |
| XV-P5-15-BC08 | 181 | AATGATACGGCGACCACCGAGATCTACACCACTTCATACACTCTTTCC |
| XV-P5-15-BC09 | 182 | AATGATACGGCGACCACCGAGATCTACACAATACCATACACTCTTTCC |
| XV-P5-15-BC10 | 183 | AATGATACGGCGACCACCGAGATCTACACATGAATTAACACTCTTTCC |
| XV-P5-15-BC11 | 184 | AATGATACGGCGACCACCGAGATCTACACTGCTTCACACACTCTTTCC |
| XV-P5-15-BC12 | 185 | AATGATACGGCGACCACCGAGATCTACACATCCTTAAACACTCTTTCC |
| XV-P5-15-BC13 | 186 | AATGATACGGCGACCACCGAGATCTACACGCTAGCAGACACTCTTTCC |
| XV-P5-15-BC14 | 187 | AATGATACGGCGACCACCGAGATCTACACTAGCATTGACACTCTTTCC |
| XV-P5-15-BC15 | 188 | AATGATACGGCGACCACCGAGATCTACACCTACATTGACACTCTTTCC |
| XV-P5-15-BC16 | 189 | AATGATACGGCGACCACCGAGATCTACACTCATTCGAACACTCTTTCC |
| XV-P5-15-BC17 | 190 | AATGATACGGCGACCACCGAGATCTACACTTAGCCAGACACTCTTTCC |
| XV-P5-15-BC18 | 191 | AATGATACGGCGACCACCGAGATCTACACGAACTTCGACACTCTTTCC |
| XV-P5-15-BC19 | 192 | AATGATACGGCGACCACCGAGATCTACACGACGGTTAACACTCTTTCC |
| XV-P5-15-BC20 | 193 | AATGATACGGCGACCACCGAGATCTACACCAAGCTTAACACTCTTTCC |
| XV-P5-15-BC21 | 194 | AATGATACGGCGACCACCGAGATCTACACTCAGGCTTACACTCTTTCC |
| XV-P5-15-BC22 | 195 | AATGATACGGCGACCACCGAGATCTACACTACTCCAGACACTCTTTCC |
| XV-P5-15-BC23 | 227 | AATGATACGGCGACCACCGAGATCTACACGCTTCCTAACACTCTTTCC |
| XV-P5-15-BC24 | 228 | AATGATACGGCGACCACCGAGATCTACACTTCTTGGCACACTCTTTCC |

### Other Embodiments

From the foregoing description, it will be apparent that variations and modifications may be made to the embodiments of the disclosure described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for preparation of a mitochondrial cDNA sequencing library, the method comprising:
a) preparing a cDNA library derived from a cell comprising a mitochondrion, wherein the cDNA library comprises polynucleotides, each polynucleotide comprising in order from 5′ to 3′ the nucleotide sequence CTACACGACGCTCTTCCGATCT (SEQ ID NO: 1), or a variant thereof with up to 5 nucleotide alterations, and a full-length cDNA polynucleotide, wherein the 5′ end of the cDNA polynucleotide corresponds to the 5′ end of an mRNA molecule derived from the cell;
b) contacting the cDNA library with a DNA polymerase, a first forward primer, and a first reverse primer under conditions sufficient to amplify the polynucleotides present in the cDNA library in a first PCR reaction,
wherein the first forward primer comprises the sequence ACACTCTTTCCCTACACGACGCTCTTCCGATCT (SEQ ID NO: 2), or a variant thereof with up to 5 [[Jo]] nucleotide alterations, and
wherein the first reverse primer comprises in order from 5′ to 3′ the nucleotide sequence CACCCGAGAATTCCA (SEQ ID NO: 3), or a variant thereof with up to 5 nucleotide alterations, and a sequence complementary to a mitochondrial cDNA polynucleotide, to yield first PCR amplicons; and
c) contacting the first PCR amplicons with a DNA polymerase, a second forward primer, and a second reverse primer under conditions sufficient to amplify the first PCR amplicons in a second PCR reaction,
wherein the second forward primer for the second PCR reaction comprises in order from 5′ to 3′ the nucleotide sequence AATGATACGGCGACCACCGAGATCTACAC (SEQ ID NO: 4 or a variant thereof with up to 5 nucleotide alterations, and the nucleotide sequence ACACTCTTTCC (SEQ ID NO: 5), or a variant thereof with up to 5 nucleotide alterations, and wherein the second reverse primer comprises in order from 5′ end to 3′ end the nucleotide sequence CAAGCAGAAGACGGCATACGAGAT (SEQ ID NO: 6), or a variant thereof with up to 5 nucleotide alterations, and the nucleotide sequence GTGACTGGAGTTCCTTGGCACCCGAGAATTCCA (SEQ ID NO: 7), or a variant thereof with up to 5 nucleotide alterations, thereby preparing the mitochondrial cDNA sequencing library.

2. The method of claim 1, wherein step b) further comprises two or more first PCR reactions, wherein one of the first PCR reactions comprises using a first unique set of first reverse primers comprising nucleotide sequences corresponding to a first RNA sequencing primer mixture of Table 2 and wherein another of the first PCR reactions comprises using a second unique set of first reverse primers comprising nucleotide sequences corresponding to a second RNA sequencing primer mixture of Table 2.

3. The method of claim 2, wherein step b) comprises twelve first PCR reactions, wherein each of the twelve first PCR reactions comprises using a unique set of first reverse primers, each unique set of reverse primers comprising nucleotide sequences corresponding to an RNA sequencing primer mixture of Table 2.

4. The method of claim 3, further comprising pooling the first PCR amplicons from each of the twelve first PCR reactions at the following volumetric ratios: 40 volumetric units of each of the first PCR amplicons corresponding to primer mixes 1-9, 12 volumetric units of the first PCR amplicon mixture corresponding to primer mix 10, 8 volumetric units of the first PCR amplicon mixture corresponding to each of primer mixes 11 and 12.

5. The method of claim 1, wherein the amount of the cDNA library used in the first PCR reaction is from about 1 ng to about 25 ng.

6. The method of claim 1, wherein the polynucleotide of step a) further comprises a Unique Molecular Identifier (UMI) sequence that identifies an mRNA molecule.

7. The method of claim 6, wherein the Unique Molecular Identifier (UMI) sequence identifies PCR duplicates present in the mitochondrial cDNA sequencing library.

8. The method of claim 1, wherein the cell of step a) is an isolated cell present in an aqueous solution-in-oil emulsion, where each aqueous solution droplet within the emulsion contains a single cell, a gel bead, and reagents appropriate for the preparation of a cDNA library within each aqueous solution droplet.

9. The method of claim 1, wherein the polynucleotide of step a) further comprises a barcode sequence identifying the cell from which the polynucleotide was derived.

10. The method of claim 1, wherein the molar ratio of the first forward primer to the first reverse primer (F:R) is from about 1:2 to about 1:3; and/or
the molar ratio of the second forward primer to the second reverse primer is from about 1:4 to about 1:6.

11. The method of claim 1, wherein the second forward primer and second reverse primer each comprises a nucleotide sequence selected from those listed in Table 3.

12. A method for sequencing mitochondrial cDNA, the method comprising:
a) preparing a mitochondrial cDNA library derived from a cell comprising a mitochondrion, wherein the mitochondrial cDNA library comprises polynucleotides, each polynucleotide comprising in order from 5′ to 3′ the nucleotide sequence CTACACGACGCTCTTCCGATCT (SEQ ID NO: 1), or a variant thereof with up to 5 nucleotide alterations, and a full-length cDNA polynucleotide, wherein the 5′ end of the cDNA polynucleotide corresponds to the 5′ end of an mRNA molecule derived from the cell;
b) contacting the mitochondrial cDNA library with a DNA polymerase, a first forward primer, and a first reverse primer under conditions sufficient to amplify the polynucleotides present in the mitochondrial cDNA library in a first PCR reaction,
wherein the first forward primer comprises the sequence ACACTCTTTCCCTACACGACGCTCTTCCGATCT (SEQ ID NO: 2), or a variant thereof with up to 5 nucleotide alterations, and
wherein the first reverse primer comprises in order from 5′ to 3′ the nucleotide sequence CACCCGAGAATTCCA (SEQ ID NO: 3), or a variant thereof with up to 5 nucleotide alterations, and a sequence complementary to a mitochondrial cDNA polynucleotide, to yield first PCR amplicons; and
c) contacting the first PCR amplicons with a DNA polymerase, a second forward primer, and a second reverse primer under conditions sufficient to amplify the first PCR amplicons in a second PCR reaction,
wherein the second forward primer for the second PCR reaction comprises in order from 5′ to 3′ the nucleotide sequence AATGATACGGCGACCACCGAGATCTACAC (SEQ ID NO: 4 or a variant thereof with up to 5 nucleotide alterations, and the nucleotide sequence ACACTCTTTCC (SEQ ID NO: 5), or a variant thereof with up to 5 nucleotide alterations, and
wherein the second reverse primer comprises in order from 5′ end to 3′ end the nucleotide sequence CAAGCAGAAGACGGCATACGAGAT (SEQ ID NO: 6), or a variant thereof with up to 5 nucleotide alterations, and the nucleotide sequence GTGACTGGAGTTCCTTGGCACCCGAGAATTCCA (SEQ ID NO: 7), or a variant thereof with up to 5 nucleotide alterations; and
d) sequencing the mitochondrial cDNA sequencing library to yield sequencing data.

13. The method of claim 12, wherein at least 90% of sequence reads map to the mitochondrial transcriptome.

14. The method of claim 12, wherein the method detects alterations in the sequence of a mitochondrial gene relative to a wild-type mitochondrial gene.

15. The method of claim 14, wherein the mitochondrial gene is ATP8, ND4L, or ND6.

16. The method claim 12, further comprising:
detecting a clone of cells by analyzing the sequence data to identify single nucleotide variants (SNVs) and/or copy number variants (CNVs) in the sequence data,
wherein the mitochondrial mRNA is from a biological sample obtained from a subject comprising cells.

17. The method of claim 16, further comprising using the identified SNVs and/or CNVs in the sequence data to cluster the sequence data and identify clonal and/or subclonal cell populations in the biological sample.

18. The method of claim 12, further comprising:
characterizing a neoplasia in a subject at at least two time points by analyzing the sequence data to identify single nucleotide variants (SNVs) and/or copy number variants (CNVs) in the sequence data,
wherein the mitochondrial mRNA is from a biological sample obtained from a subject having the neoplasia, and wherein the biological sample comprises neoplastic cells.

19. A kit suitable for use in the preparation of a mitochondrial cDNA sequencing library, wherein the kit comprises:
a) a first primer consisting of the nucleotide sequence ACACTCTTTCCCTACACGACGCTCTTCCGATCT (SEQ ID NO: 2), or a variant thereof with up to 5 nucleotide alterations,
b) a second primer consisting of in order from 5′ to 3′ the nucleotide sequence AATGATACGGCGACCACCGAGATCTACAC (SEQ ID NO: 4) or a variant thereof with up to 5 nucleotide alterations, a first indexing primer, and the nucleotide sequence ACACTCTTTCC (SEQ ID NO: 5), or a variant thereof with up to 5 nucleotide alterations,
c) a third primer consisting of in order from 5′ to 3′ the nucleotide sequence CACCCGAGAATTCCA (SEQ ID NO: 3), or a variant thereof with up to 5 nucleotide alterations, and a sequence complementary to a mitochondrial cDNA polynucleotide, and/or
d) a fourth primer consisting of in order from 5′ to 3′ the nucleotide sequence CAAGCAGAAGACGGCATACGAGAT (SEQ ID NO: 6), or a variant thereof with up to 5 nucleotide alterations, a second indexing primer, and the nucleotide sequence GTGACTGGAGTTCCTTGGCACCCGAGAATTCCA (SEQ ID NO: 7), or a variant thereof with up to 5 nucleotide alterations.

20. The kit of claim 19, wherein the third primer contains a nucleotide sequence selected from those RNA sequencing primer sequences listed in Table 2.


SEQUENCE LISTING (228 sequences)

SEQ ID NO: 1 - DNA, 22 bp, synthetic construct
  source 1..22: mol_type = other DNA
ctacacgacgctcttccgatct

SEQ ID NO: 2 - DNA, 33 bp, synthetic construct
  source 1..33: mol_type = other DNA
acactctttccctacacgacgctcttccgatct

SEQ ID NO: 3 - DNA, 15 bp, synthetic construct
  source 1..15: mol_type = other DNA
cacccgagaattcca

SEQ ID NO: 4 - DNA, 29 bp, synthetic construct
  source 1..29: mol_type = other DNA
aatgatacggcgaccaccgagatctacac

SEQ ID NO: 5 - DNA, 11 bp, synthetic construct
  source 1..11: mol_type = other DNA
acactctttcc

SEQ ID NO: 6 - DNA, 24 bp, synthetic construct
  source 1..24: mol_type = other DNA
caagcagaagacggcatacgagat

SEQ ID NO: 7 - DNA, 33 bp, synthetic construct
  source 1..33: mol_type = other DNA
gtgactggagttccttggcacccgagaattcca

SEQ ID NO: 8 - DNA, 13 bp, synthetic construct
  source 1..13: mol_type = other DNA
tttcttatatggg

SEQ ID NO: 9 - DNA, 25 bp, synthetic construct
  source 1..25: mol_type = other DNA
gtactctgcgttgataccactgctt

SEQ ID NO: 10 - DNA, 61 bp, synthetic construct
  source 1..61: mol_type = other DNA
  misc_difference 23: note = a, c, t, g, unknown or other
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
  misc_difference 42: note = a, c, t, g, unknown or other
  misc_difference 43: note = a, c, t, g, unknown or other
  misc_difference 44: note = a, c, t, g, unknown or other
  misc_difference 45: note = a, c, t, g, unknown or other
  misc_difference 46: note = a, c, t, g, unknown or other
  misc_difference 47: note = a, c, t, g, unknown or other
  misc_difference 48: note = a, c, t, g, unknown or other
ctacacgacgctcttccgatctnnnnnnnnnnnnnnnnnnnnnnnnnntttcttatatggg

SEQ ID NO: 11 - DNA, 57 bp, synthetic construct
  source 1..57: mol_type = other DNA
  misc_difference 57: note = a, c, t, g, unknown or other
aagcagtggtatcaacgcagagtacttttttttttttttttttttttttttttttvn

SEQ ID NO: 12 - DNA, 29 bp, synthetic construct
  source 1..29: mol_type = other DNA
aatgatacggcgaccaccgagatctacac

SEQ ID NO: 13 - DNA, 11 bp, synthetic construct
  source 1..11: mol_type = other DNA
acactctttcc

SEQ ID NO: 14 - DNA, 33 bp, synthetic construct; identical to SEQ ID NO: 7
  source 1..33: mol_type = other DNA

SEQ ID NO: 15 - DNA, 24 bp, synthetic construct
  source 1..24: mol_type = other DNA
caagcagaagacggcatacgagat

SEQ ID NO: 16 - DNA, 20 bp, synthetic construct
  source 1..20: mol_type = other DNA
ggagcgttatggagtggaag

SEQ ID NO: 17 - DNA, 21 bp, synthetic construct
  source 1..21: mol_type = other DNA
tggttctcagggtttgttata

SEQ ID NO: 18 - DNA, 21 bp, synthetic construct
  source 1..21: mol_type = other DNA
taagagggagtgggtgttgag

SEQ ID NO: 19 - DNA, 20 bp, synthetic construct
  source 1..20: mol_type = other DNA
gagtgatgtgggcgattgat

SEQ ID NO: 20 - DNA, 20 bp, synthetic construct
  source 1..20: mol_type = other DNA
gtttgggttgtggctcagtg

SEQ ID NO: 21 - DNA, 21 bp, synthetic construct
  source 1..21: mol_type = other DNA
gtaggggtaaaaggagggcaa

SEQ ID NO: 22 - DNA, 21 bp, synthetic construct
  source 1..21: mol_type = other DNA
gatactcctcaatagccatcg

SEQ ID NO: 23 - DNA, 23 bp, synthetic construct
  source 1..23: mol_type = other DNA
agttgccaaagcctccgattatg

SEQ ID NO: 24 - DNA, 20 bp, synthetic construct
  source 1..20: mol_type = other DNA
gaagagcgatggtgagagct

SEQ ID NO: 25 - DNA, 20 bp, synthetic construct
  source 1..20: mol_type = other DNA
gataagtggcgttggcttgc

SEQ ID NO: 26 - DNA, 21 bp, synthetic construct
  source 1..21: mol_type = other DNA
agtccgaggaggttagttgtg

SEQ ID NO: 27 - DNA, 20 bp, synthetic construct
  source 1..20: mol_type = other DNA
ttggttatggttcattgtcc

SEQ ID NO: 28 - DNA, 24 bp, synthetic construct
  source 1..24: mol_type = other DNA
aggtttaggttatgtacgtagtct

SEQ ID NO: 29 - DNA, 20 bp, synthetic construct
  source 1..20: mol_type = other DNA
gtaagggagggatcgttgac

SEQ ID NO: 30 - DNA, 20 bp, synthetic construct
  source 1..20: mol_type = other DNA
cacccacagcaccaatccta

SEQ ID NO: 31 - DNA, 20 bp, synthetic construct
  source 1..20: mol_type = other DNA
ttcggttcagtctaatcctt

SEQ ID NO: 32 - DNA, 19 bp, synthetic construct
  source 1..19: mol_type = other DNA
gccagtgccctcctaattg

SEQ ID NO: 33 - DNA, 20 bp, synthetic construct
  source 1..20: mol_type = other DNA
agcaggaggataatgccgat

SEQ ID NO: 34 - DNA, 24 bp, synthetic construct
  source 1..24: mol_type = other DNA
gcggtaactaagattagtatggta

SEQ ID NO: 35 - DNA, 20 bp, synthetic construct
  source 1..20: mol_type = other DNA
gatggttaggtctacggagg

SEQ ID NO: 36 - DNA, 24 bp, synthetic construct
  source 1..24: mol_type = other DNA
gctgatggtttcgataataactag

SEQ ID NO: 37 - DNA, 21 bp, synthetic construct
  source 1..21: mol_type = other DNA
ttgagagagtgaggagaaggc

SEQ ID NO: 38 - DNA, 20 bp, synthetic construct
  source 1..20: mol_type = other DNA
gagtaggggaagggagccta

SEQ ID NO: 39 - DNA, 22 bp, synthetic construct
  source 1..22: mol_type = other DNA
gtggagaggttaaaggagccac

SEQ ID NO: 40 - DNA, 21 bp, synthetic construct
  source 1..21: mol_type = other DNA
cctaatgtggggacagctcat

SEQ ID NO: 41 - DNA, 22 bp, synthetic construct
  source 1..22: mol_type = other DNA
cgagcaatctcaattacaatat

SEQ ID NO: 42 - DNA, 20 bp, synthetic construct
  source 1..20: mol_type = other DNA
aatcgtgtgagggtgggact

SEQ ID NO: 43 - DNA, 20 bp, synthetic construct
  source 1..20: mol_type = other DNA
gtcggaaatggtgaagggag

SEQ ID NO: 44 - DNA, 21 bp, synthetic construct
  source 1..21: mol_type = other DNA
ggataaatcatgctaaggcga

SEQ ID NO: 45 - DNA, 20 bp, synthetic construct
  source 1..20: mol_type = other DNA
ggtcgaagaaggtggtgttg

SEQ ID NO: 46 - DNA, 20 bp, synthetic construct
  source 1..20: mol_type = other DNA
ggcttcgacatgggctttag

SEQ ID NO: 47 - DNA, 22 bp, synthetic construct
  source 1..22: mol_type = other DNA
tgtcgtgcaggtagaggcttac

SEQ ID NO: 48 - DNA, 21 bp, synthetic construct
  source 1..21: mol_type = other DNA
tgcgagatagtagtagggtcg

SEQ ID NO: 49 - DNA, 20 bp, synthetic construct
  source 1..20: mol_type = other DNA
gacgatgggcatgaaactgt

SEQ ID NO: 50 - DNA, 20 bp, synthetic construct
  source 1..20: mol_type = other DNA
ggaagagaagtaagccgagg

SEQ ID NO: 51 - DNA, 22 bp, synthetic construct
  source 1..22: mol_type = other DNA
agattgtagtggtgagggtgtt

SEQ ID NO: 52 - DNA, 21 bp, synthetic construct
  source 1..21: mol_type = other DNA
ttgagaatgagtgtgaggcgt

SEQ ID NO: 53 - DNA, 23 bp, synthetic construct
  source 1..23: mol_type = other DNA
gatggagacatacagaaatagtc

SEQ ID NO: 54 - DNA, 21 bp, synthetic construct
  source 1..21: mol_type = other DNA
gtggaagcggatgagtaagaa

SEQ ID NO: 55 - DNA, 23 bp, synthetic construct
  source 1..23: mol_type = other DNA
tcacacgataaaccctaggaagc

SEQ ID NO: 56 - DNA, 23 bp, synthetic construct
  source 1..23: mol_type = other DNA
ggagtagtgtgattgaggtggag

SEQ ID NO: 57 - DNA, 21 bp, synthetic construct
  source 1..21: mol_type = other DNA
atggggattattgctaggatg

SEQ ID NO: 58 - DNA, 22 bp, synthetic construct
  source 1..22: mol_type = other DNA
gggaatgctggagattgtaatg

SEQ ID NO: 59 - DNA, 20 bp, synthetic construct
  source 1..20: mol_type = other DNA
gcgattatgagaatgactgc

SEQ ID NO: 60 - DNA, 21 bp, synthetic construct
  source 1..21: mol_type = other DNA
agataggtaggagtagcgtgg

SEQ ID NO: 61 - DNA, 20 bp, synthetic construct
  source 1..20: mol_type = other DNA
taatacaatgccagtcaggc

SEQ ID NO: 62 - DNA, 23 bp, synthetic construct
  source 1..23: mol_type = other DNA
gacctgtgggtttgttaggtact

SEQ ID NO: 63 - DNA, 20 bp, synthetic construct
  source 1..20: mol_type = other DNA
ttgtcagggaggtagcgatg

SEQ ID NO: 64 - DNA, 23 bp, synthetic construct
  source 1..23: mol_type = other DNA
ctccctaattgaaaacaaaatac

SEQ ID NO: 65 - DNA, 22 bp, synthetic construct
  source 1..22: mol_type = other DNA
gatcaggagaacgtggttacta

SEQ ID NO: 66 - DNA, 20 bp, synthetic construct
  source 1..20: mol_type = other DNA
ggtagtccgagtaacgtcgg

SEQ ID NO: 67 - DNA, 20 bp, synthetic construct
  source 1..20: mol_type = other DNA
ctaggaaagtgacagcgagg

SEQ ID NO: 68 - DNA, 21 bp, synthetic construct
  source 1..21: mol_type = other DNA
atcgggatgtcctgatccaac

SEQ ID NO: 69 - DNA, 22 bp, synthetic construct
  source 1..22: mol_type = other DNA
gaaaacccggtaatgatgtcgg

SEQ ID NO: 70 - DNA, 20 bp, synthetic construct
  source 1..20: mol_type = other DNA
ttatgtatacgggttcttcg

SEQ ID NO: 71 - DNA, 20 bp, synthetic construct
  source 1..20: mol_type = other DNA
tgtgcggtgtgtgatgctag

SEQ ID NO: 72 - DNA, 22 bp, synthetic construct
  source 1..22: mol_type = other DNA
gttcttgggtgggtgtgggtat

SEQ ID NO: 73 - DNA, 21 bp, synthetic construct
  source 1..21: mol_type = other DNA
gtgattaggagtagggttagg

SEQ ID NO: 74 - DNA, 20 bp, synthetic construct
  source 1..20: mol_type = other DNA
gcggtgcctctaatactggt

SEQ ID NO: 75 - DNA, 21 bp, synthetic construct
  source 1..21: mol_type = other DNA
gcactttccagtacacttacc

SEQ ID NO: 76 - DNA, 22 bp, synthetic construct
  source 1..22: mol_type = other DNA
cggagcacataaatagtatggc

SEQ ID NO: 77 - DNA, 20 bp, synthetic construct
  source 1..20: mol_type = other DNA
atagggtgatagattggtcc

SEQ ID NO: 78 - DNA, 20 bp, synthetic construct
  source 1..20: mol_type = other DNA
tgtagcccatttcttgccac

SEQ ID NO: 79 - DNA, 25 bp, synthetic construct
  source 1..25: mol_type = other DNA
gttgaactaagattctatcttggga

SEQ ID NO: 80 - DNA, 20 bp, synthetic construct
  source 1..20: mol_type = other DNA
gtagtgttctggcgagcagt

SEQ ID NO: 81 - DNA, 21 bp, synthetic construct
  source 1..21: mol_type = other DNA
cttagctttggctctccttgc

SEQ ID NO: 82 - DNA, 20 bp, synthetic construct
  source 1..20: mol_type = other DNA
tgggttaatcgtgtgaccgc

SEQ ID NO: 83 - DNA, 35 bp, synthetic construct
  source 1..35: mol_type = other DNA
cacccgagaattccaggagcgttatggagtggaag

SEQ ID NO: 84 - DNA, 36 bp, synthetic construct
  source 1..36: mol_type = other DNA
cacccgagaattccatggttctcagggtttgttata

SEQ ID NO: 85 - DNA, 36 bp, synthetic construct
  source 1..36: mol_type = other DNA
cacccgagaattccataagagggagtgggtgttgag

SEQ ID NO: 86 - DNA, 35 bp, synthetic construct
  source 1..35: mol_type = other DNA
cacccgagaattccagagtgatgtgggcgattgat

SEQ ID NO: 87 - DNA, 35 bp, synthetic construct
  source 1..35: mol_type = other DNA
cacccgagaattccagtttgggttgtggctcagtg

SEQ ID NO: 88 - DNA, 36 bp, synthetic construct
  source 1..36: mol_type = other DNA
cacccgagaattccagtaggggtaaaaggagggcaa

SEQ ID NO: 89 - DNA, 36 bp, synthetic construct
  source 1..36: mol_type = other DNA
cacccgagaattccagatactcctcaatagccatcg

SEQ ID NO: 90 - DNA, 38 bp, synthetic construct
  source 1..38: mol_type = other DNA
cacccgagaattccaagttgccaaagcctccgattatg

SEQ ID NO: 91 - DNA, 35 bp, synthetic construct
  source 1..35: mol_type = other DNA
cacccgagaattccagaagagcgatggtgagagct

SEQ ID NO: 92 - DNA, 35 bp, synthetic construct
  source 1..35: mol_type = other DNA
cacccgagaattccagataagtggcgttggcttgc

SEQ ID NO: 93 - DNA, 36 bp, synthetic construct
  source 1..36: mol_type = other DNA
cacccgagaattccaagtccgaggaggttagttgtg

SEQ ID NO: 94 - DNA, 35 bp, synthetic construct
  source 1..35: mol_type = other DNA
cacccgagaattccattggttatggttcattgtcc

SEQ ID NO: 95 - DNA, 39 bp, synthetic construct
  source 1..39: mol_type = other DNA
cacccgagaattccaaggtttaggttatgtacgtagtct

SEQ ID NO: 96 - DNA, 35 bp, synthetic construct
  source 1..35: mol_type = other DNA
cacccgagaattccagtaagggagggatcgttgac

SEQ ID NO: 97 - DNA, 35 bp, synthetic construct
  source 1..35: mol_type = other DNA
cacccgagaattccacacccacagcaccaatccta

SEQ ID NO: 98 - DNA, 35 bp, synthetic construct
  source 1..35: mol_type = other DNA
cacccgagaattccattcggttcagtctaatcctt

SEQ ID NO: 99 - DNA, 34 bp, synthetic construct
  source 1..34: mol_type = other DNA
cacccgagaattccagccagtgccctcctaattg

SEQ ID NO: 100 - DNA, 35 bp, synthetic construct
  source 1..35: mol_type = other DNA
cacccgagaattccaagcaggaggataatgccgat

SEQ ID NO: 101 - DNA, 39 bp, synthetic construct
  source 1..39: mol_type = other DNA
cacccgagaattccagcggtaactaagattagtatggta

SEQ ID NO: 102 - DNA, 35 bp, synthetic construct
  source 1..35: mol_type = other DNA
cacccgagaattccagatggttaggtctacggagg

SEQ ID NO: 103 - DNA, 39 bp, synthetic construct
  source 1..39: mol_type = other DNA
cacccgagaattccagctgatggtttcgataataactag

SEQ ID NO: 104 - DNA, 36 bp, synthetic construct
  source 1..36: mol_type = other DNA
cacccgagaattccattgagagagtgaggagaaggc

SEQ ID NO: 105 - DNA, 35 bp, synthetic construct
  source 1..35: mol_type = other DNA
cacccgagaattccagagtaggggaagggagccta

SEQ ID NO: 106 - DNA, 37 bp, synthetic construct
  source 1..37: mol_type = other DNA
cacccgagaattccagtggagaggttaaaggagccac

SEQ ID NO: 107 - DNA, 36 bp, synthetic construct
  source 1..36: mol_type = other DNA
cacccgagaattccacctaatgtggggacagctcat

SEQ ID NO: 108 - DNA, 37 bp, synthetic construct
  source 1..37: mol_type = other DNA
cacccgagaattccacgagcaatctcaattacaatat

SEQ ID NO: 109 - DNA, 35 bp, synthetic construct
  source 1..35: mol_type = other DNA
cacccgagaattccaaatcgtgtgagggtgggact

SEQ ID NO: 110 - DNA, 35 bp, synthetic construct
  source 1..35: mol_type = other DNA
cacccgagaattccagtcggaaatggtgaagggag

SEQ ID NO: 111 - DNA, 36 bp, synthetic construct
  source 1..36: mol_type = other DNA
cacccgagaattccaggataaatcatgctaaggcga

SEQ ID NO: 112 - DNA, 35 bp, synthetic construct
  source 1..35: mol_type = other DNA
cacccgagaattccaggtcgaagaaggtggtgttg

SEQ ID NO: 113 - DNA, 35 bp, synthetic construct
  source 1..35: mol_type = other DNA
cacccgagaattccaggcttcgacatgggctttag

SEQ ID NO: 114 - DNA, 37 bp, synthetic construct
  source 1..37: mol_type = other DNA
cacccgagaattccatgtcgtgcaggtagaggcttac

SEQ ID NO: 115 - DNA, 36 bp, synthetic construct
  source 1..36: mol_type = other DNA
cacccgagaattccatgcgagatagtagtagggtcg

SEQ ID NO: 116 - DNA, 35 bp, synthetic construct
  source 1..35: mol_type = other DNA
cacccgagaattccagacgatgggcatgaaactgt

SEQ ID NO: 117 - DNA, 35 bp, synthetic construct
  source 1..35: mol_type = other DNA
cacccgagaattccaggaagagaagtaagccgagg

SEQ ID NO: 118 - DNA, 37 bp, synthetic construct
  source 1..37: mol_type = other DNA
cacccgagaattccaagattgtagtggtgagggtgtt

SEQ ID NO: 119 - DNA, 36 bp, synthetic construct
  source 1..36: mol_type = other DNA
cacccgagaattccattgagaatgagtgtgaggcgt

SEQ ID NO: 120 - DNA, 38 bp, synthetic construct
  source 1..38: mol_type = other DNA
cacccgagaattccagatggagacatacagaaatagtc

SEQ ID NO: 121 - DNA, 36 bp, synthetic construct
  source 1..36: mol_type = other DNA
cacccgagaattccagtggaagcggatgagtaagaa

SEQ ID NO: 122 - DNA, 38 bp, synthetic construct
  source 1..38: mol_type = other DNA
cacccgagaattccatcacacgataaaccctaggaagc

SEQ ID NO: 123 - DNA, 38 bp, synthetic construct
  source 1..38: mol_type = other DNA
cacccgagaattccaggagtagtgtgattgaggtggag

SEQ ID NO: 124 - DNA, 36 bp, synthetic construct
  source 1..36: mol_type = other DNA
cacccgagaattccaatggggattattgctaggatg

SEQ ID NO: 125 - DNA, 37 bp, synthetic construct
  source 1..37: mol_type = other DNA
cacccgagaattccagggaatgctggagattgtaatg

SEQ ID NO: 126 - DNA, 35 bp, synthetic construct
  source 1..35: mol_type = other DNA
cacccgagaattccagcgattatgagaatgactgc

SEQ ID NO: 127 - DNA, 36 bp, synthetic construct
  source 1..36: mol_type = other DNA
cacccgagaattccaagataggtaggagtagcgtgg

SEQ ID NO: 128 - DNA, 35 bp, synthetic construct
  source 1..35: mol_type = other DNA
cacccgagaattccataatacaatgccagtcaggc

SEQ ID NO: 129 - DNA, 38 bp, synthetic construct
  source 1..38: mol_type = other DNA
cacccgagaattccagacctgtgggtttgttaggtact

SEQ ID NO: 130 - DNA, 35 bp, synthetic construct
  source 1..35: mol_type = other DNA
cacccgagaattccattgtcagggaggtagcgatg

SEQ ID NO: 131 - DNA, 38 bp, synthetic construct
  source 1..38: mol_type = other DNA
cacccgagaattccactccctaattgaaaacaaaatac

SEQ ID NO: 132 - DNA, 37 bp, synthetic construct
  source 1..37: mol_type = other DNA
cacccgagaattccagatcaggagaacgtggttacta

SEQ ID NO: 133 - DNA, 35 bp, synthetic construct
  source 1..35: mol_type = other DNA
cacccgagaattccaggtagtccgagtaacgtcgg

SEQ ID NO: 134 - DNA, 35 bp, synthetic construct
  source 1..35: mol_type = other DNA
cacccgagaattccactaggaaagtgacagcgagg

SEQ ID NO: 135 - DNA, 36 bp, synthetic construct
  source 1..36: mol_type = other DNA
cacccgagaattccaatcgggatgtcctgatccaac

SEQ ID NO: 136 - DNA, 37 bp, synthetic construct
  source 1..37: mol_type = other DNA
cacccgagaattccagaaaacccggtaatgatgtcgg

SEQ ID NO: 137 - DNA, 35 bp, synthetic construct
  source 1..35: mol_type = other DNA
cacccgagaattccattatgtatacgggttcttcg

SEQ ID NO: 138 - DNA, 35 bp, synthetic construct
  source 1..35: mol_type = other DNA
cacccgagaattccatgtgcggtgtgtgatgctag

SEQ ID NO: 139 - DNA, 37 bp, synthetic construct
  source 1..37: mol_type = other DNA
cacccgagaattccagttcttgggtgggtgtgggtat

SEQ ID NO: 140 - DNA, 36 bp, synthetic construct
  source 1..36: mol_type = other DNA
cacccgagaattccagtgattaggagtagggttagg

SEQ ID NO: 141 - DNA, 35 bp, synthetic construct
  source 1..35: mol_type = other DNA
cacccgagaattccagcggtgcctctaatactggt

SEQ ID NO: 142 - DNA, 36 bp, synthetic construct
  source 1..36: mol_type = other DNA
cacccgagaattccagcactttccagtacacttacc

SEQ ID NO: 143 - DNA, 37 bp, synthetic construct
  source 1..37: mol_type = other DNA
cacccgagaattccacggagcacataaatagtatggc

SEQ ID NO: 144 - DNA, 35 bp, synthetic construct
  source 1..35: mol_type = other DNA
cacccgagaattccaatagggtgatagattggtcc

SEQ ID NO: 145 - DNA, 35 bp, synthetic construct
  source 1..35: mol_type = other DNA
cacccgagaattccatgtagcccatttcttgccac

SEQ ID NO: 146 - DNA, 40 bp, synthetic construct
  source 1..40: mol_type = other DNA
cacccgagaattccagttgaactaagattctatcttggga

SEQ ID NO: 147 - DNA, 35 bp, synthetic construct
  source 1..35: mol_type = other DNA
cacccgagaattccagtagtgttctggcgagcagt

SEQ ID NO: 148 - DNA, 36 bp, synthetic construct
  source 1..36: mol_type = other DNA
cacccgagaattccacttagctttggctctccttgc

SEQ ID NO: 149 - DNA, 35 bp, synthetic construct
  source 1..35: mol_type = other DNA
cacccgagaattccatgggttaatcgtgtgaccgc

SEQ ID NO: 150 - DNA, 65 bp, synthetic construct
  source 1..65: mol_type = other DNA
caagcagaagacggcatacgagataagtagaggtgactggagttccttggcacccgagaattcca

SEQ ID NO: 151 - DNA, 65 bp, synthetic construct
  source 1..65: mol_type = other DNA
caagcagaagacggcatacgagatggtccagagtgactggagttccttggcacccgagaattcca

SEQ ID NO: 152 - DNA, 65 bp, synthetic construct
  source 1..65: mol_type = other DNA
caagcagaagacggcatacgagatgcacatctgtgactggagttccttggcacccgagaattcca

SEQ ID NO: 153 - DNA, 65 bp, synthetic construct
  source 1..65: mol_type = other DNA
caagcagaagacggcatacgagatttcgctgagtgactggagttccttggcacccgagaattcca

SEQ ID NO: 154 - DNA, 65 bp, synthetic construct
  source 1..65: mol_type = other DNA
caagcagaagacggcatacgagatagcaattcgtgactggagttccttggcacccgagaattcca

SEQ ID NO: 155 - DNA, 65 bp, synthetic construct
  source 1..65: mol_type = other DNA
caagcagaagacggcatacgagatcacatcctgtgactggagttccttggcacccgagaattcca

SEQ ID NO: 156 - DNA, 65 bp, synthetic construct
  source 1..65: mol_type = other DNA
caagcagaagacggcatacgagatccagttaggtgactggagttccttggcacccgagaattcca

SEQ ID NO: 157 - DNA, 65 bp, synthetic construct
  source 1..65: mol_type = other DNA
caagcagaagacggcatacgagataaggatgtgtgactggagttccttggcacccgagaattcca

SEQ ID NO: 158 - DNA, 65 bp, synthetic construct
  source 1..65: mol_type = other DNA
caagcagaagacggcatacgagatacacgatcgtgactggagttccttggcacccgagaattcca

SEQ ID NO: 159 - DNA, 65 bp, synthetic construct
  source 1..65: mol_type = other DNA
caagcagaagacggcatacgagatcatgcttagtgactggagttccttggcacccgagaattcca

SEQ ID NO: 160 - DNA, 65 bp, synthetic construct
  source 1..65: mol_type = other DNA
caagcagaagacggcatacgagatgtataacagtgactggagttccttggcacccgagaattcca

SEQ ID NO: 161 - DNA, 65 bp, synthetic construct
  source 1..65: mol_type = other DNA
caagcagaagacggcatacgagattgctcgacgtgactggagttccttggcacccgagaattcca

SEQ ID NO: 162 - DNA, 65 bp, synthetic construct
  source 1..65: mol_type = other DNA
caagcagaagacggcatacgagataacttgacgtgactggagttccttggcacccgagaattcca

SEQ ID NO: 163 - DNA, 65 bp, synthetic construct
  source 1..65: mol_type = other DNA
caagcagaagacggcatacgagatagttgcttgtgactggagttccttggcacccgagaattcca

SEQ ID NO: 164 - DNA, 65 bp, synthetic construct
  source 1..65: mol_type = other DNA
caagcagaagacggcatacgagattcggaatggtgactggagttccttggcacccgagaattcca

SEQ ID NO: 165 - DNA, 65 bp, synthetic construct
  source 1..65: mol_type = other DNA
caagcagaagacggcatacgagatttgagcctgtgactggagttccttggcacccgagaattcca

SEQ ID NO: 166 - DNA, 65 bp, synthetic construct
  source 1..65: mol_type = other DNA
caagcagaagacggcatacgagattgttccgagtgactggagttccttggcacccgagaattcca

SEQ ID NO: 167 - DNA, 65 bp, synthetic construct
  source 1..65: mol_type = other DNA
caagcagaagacggcatacgagataggatctagtgactggagttccttggcacccgagaattcca

SEQ ID NO: 168 - DNA, 65 bp, synthetic construct
  source 1..65: mol_type = other DNA
caagcagaagacggcatacgagatcatagcgagtgactggagttccttggcacccgagaattcca

SEQ ID NO: 169 - DNA, 65 bp, synthetic construct
  source 1..65: mol_type = other DNA
caagcagaagacggcatacgagatcctatgccgtgactggagttccttggcacccgagaattcca

SEQ ID NO: 170 - DNA, 65 bp, synthetic construct
  source 1..65: mol_type = other DNA
caagcagaagacggcatacgagattgtcggatgtgactggagttccttggcacccgagaattcca

SEQ ID NO: 171 - DNA, 65 bp, synthetic construct
  source 1..65: mol_type = other DNA
caagcagaagacggcatacgagatatagcgtcgtgactggagttccttggcacccgagaattcca

SEQ ID NO: 172 - DNA, 65 bp, synthetic construct
  source 1..65: mol_type = other DNA
caagcagaagacggcatacgagatcctaccatgtgactggagttccttggcacccgagaattcca

SEQ ID NO: 173 - DNA, 65 bp, synthetic construct
  source 1..65: mol_type = other DNA
caagcagaagacggcatacgagatattctagggtgactggagttccttggcacccgagaattcca

SEQ ID NO: 174 - DNA, 48 bp, synthetic construct
  source 1..48: mol_type = other DNA
aatgatacggcgaccaccgagatctacacatcgactgacactctttcc

SEQ ID NO: 175 - DNA, 48 bp, synthetic construct
  source 1..48: mol_type = other DNA
aatgatacggcgaccaccgagatctacaccggttcttacactctttcc

SEQ ID NO: 176 - DNA, 48 bp, synthetic construct
  source 1..48: mol_type = other DNA
aatgatacggcgaccaccgagatctacacaacctcttacactctttcc

SEQ ID NO: 177 - DNA, 48 bp, synthetic construct
  source 1..48: mol_type = other DNA
aatgatacggcgaccaccgagatctacaccgcatattacactctttcc

SEQ ID NO: 178 - DNA, 48 bp, synthetic construct
  source 1..48: mol_type = other DNA
aatgatacggcgaccaccgagatctacacctgctcctacactctttcc

SEQ ID NO: 179 - DNA, 48 bp, synthetic construct
  source 1..48: mol_type = other DNA
aatgatacggcgaccaccgagatctacacgctgcactacactctttcc

SEQ ID NO: 180 - DNA, 48 bp, synthetic construct
  source 1..48: mol_type = other DNA
aatgatacggcgaccaccgagatctacaccctgtcatacactctttcc

SEQ ID NO: 181 - DNA, 48 bp, synthetic construct
  source 1..48: mol_type = other DNA
aatgatacggcgaccaccgagatctacaccacttcatacactctttcc

SEQ ID NO: 182 - DNA, 48 bp, synthetic construct
  source 1..48: mol_type = other DNA
aatgatacggcgaccaccgagatctacacaataccatacactctttcc

SEQ ID NO: 183 - DNA, 48 bp, synthetic construct
  source 1..48: mol_type = other DNA
aatgatacggcgaccaccgagatctacacatgaattaacactctttcc

SEQ ID NO: 184 - DNA, 48 bp, synthetic construct
  source 1..48: mol_type = other DNA
aatgatacggcgaccaccgagatctacactgcttcacacactctttcc

SEQ ID NO: 185 - DNA, 48 bp, synthetic construct
  source 1..48: mol_type = other DNA
aatgatacggcgaccaccgagatctacacatccttaaacactctttcc

SEQ ID NO: 186 - DNA, 48 bp, synthetic construct
  source 1..48: mol_type = other DNA
aatgatacggcgaccaccgagatctacacgctagcagacactctttcc

SEQ ID NO: 187 - DNA, 48 bp, synthetic construct
  source 1..48: mol_type = other DNA
aatgatacggcgaccaccgagatctacactagcattgacactctttcc

SEQ ID NO: 188 - DNA, 48 bp, synthetic construct
  source 1..48: mol_type = other DNA
aatgatacggcgaccaccgagatctacacctacattgacactctttcc

SEQ ID NO: 189 - DNA, 48 bp, synthetic construct
  source 1..48: mol_type = other DNA
aatgatacggcgaccaccgagatctacactcattcgaacactctttcc

SEQ ID NO: 190 - DNA, 48 bp, synthetic construct
  source 1..48: mol_type = other DNA
aatgatacggcgaccaccgagatctacacttagccagacactctttcc

SEQ ID NO: 191 - DNA, 48 bp, synthetic construct
  source 1..48: mol_type = other DNA
aatgatacggcgaccaccgagatctacacgaacttcgacactctttcc

SEQ ID NO: 192 - DNA, 48 bp, synthetic construct
  source 1..48: mol_type = other DNA
aatgatacggcgaccaccgagatctacacgacggttaacactctttcc

SEQ ID NO: 193 - DNA, 48 bp, synthetic construct
  source 1..48: mol_type = other DNA
aatgatacggcgaccaccgagatctacaccaagcttaacactctttcc

SEQ ID NO: 194 - DNA, 48 bp, synthetic construct
  source 1..48: mol_type = other DNA
aatgatacggcgaccaccgagatctacactcaggcttacactctttcc

SEQ ID NO: 195 - DNA, 48 bp, synthetic construct
  source 1..48: mol_type = other DNA
aatgatacggcgaccaccgagatctacactactccagacactctttcc

SEQ ID NO: 196 - RNA, 20 nt, synthetic construct
  source 1..20: mol_type = other RNA
aaaaaaaaaaaaaaaaaaaa

SEQ ID NO: 197 - DNA, 61 bp, synthetic construct; identical to SEQ ID NO: 10
  source 1..61: mol_type = other DNA
  misc_difference 23: note = a, c, t, g, unknown or other
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
  misc_difference 42: note = a, c, t, g, unknown or other
  misc_difference 43: note = a, c, t, g, unknown or other
  misc_difference 44: note = a, c, t, g, unknown or other
  misc_difference 45: note = a, c, t, g, unknown or other
  misc_difference 46: note = a, c, t, g, unknown or other
  misc_difference 47: note = a, c, t, g, unknown or other
  misc_difference 48: note = a, c, t, g, unknown or other

SEQ ID NO: 198 - DNA, 25 bp, synthetic construct
  source 1..25: mol_type = other DNA
gtactctgcgttgataccactgctt

SEQ ID NO: 199 - DNA, 29 bp, synthetic construct
  source 1..29: mol_type = other DNA
aatgatacggcgaccaccgagatctacac

SEQ ID NO: 200 - DNA, 33 bp, synthetic construct; identical to SEQ ID NO: 2
  source 1..33: mol_type = other DNA

SEQ ID NO: 201 - DNA, 25 bp, synthetic construct
  source 1..25: mol_type = other DNA
aagcagtggtatcaacgcagagtac

SEQ ID NO: 202 - DNA, 61 bp, synthetic construct
  source 1..61: mol_type = other DNA
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_difference 18: note = a, c, t, g, unknown or other
  misc_difference 19: note = a, c, t, g, unknown or other
  misc_difference 20: note = a, c, t, g, unknown or other
  misc_difference 21: note = a, c, t, g, unknown or other
  misc_difference 22: note = a, c, t, g, unknown or other
  misc_difference 23: note = a, c, t, g, unknown or other
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
cccatataagaaannnnnnnnnnnnnnnnnnnnnnnnnnagatcggaagagcgtcgtgtag

SEQ ID NO: 203 - DNA, 29 bp, synthetic construct
  source 1..29: mol_type = other DNA
aatgatacggcgaccaccgagatctacac

SEQ ID NO: 204 - DNA, 72 bp, synthetic construct
  source 1..72: mol_type = other DNA
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
  misc_difference 42: note = a, c, t, g, unknown or other
  misc_difference 43: note = a, c, t, g, unknown or other
  misc_difference 44: note = a, c, t, g, unknown or other
  misc_difference 45: note = a, c, t, g, unknown or other
  misc_difference 46: note = a, c, t, g, unknown or other
  misc_difference 47: note = a, c, t, g, unknown or other
  misc_difference 48: note = a, c, t, g, unknown or other
  misc_difference 49: note = a, c, t, g, unknown or other
  misc_difference 50: note = a, c, t, g, unknown or other
  misc_difference 51: note = a, c, t, g, unknown or other
  misc_difference 52: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
  misc_difference 57: note = a, c, t, g, unknown or other
  misc_difference 58: note = a, c, t, g, unknown or other
  misc_difference 59: note = a, c, t, g, unknown or other
acactctttccctacacgacgctcttccgatctnnnnnnnnnnnnnnnnnnnnnnnnnntttcttatatggg

SEQ ID NO: 205 - DNA, 15 bp, synthetic construct
  source 1..15: mol_type = other DNA
tggaattctcgggtg

SEQ ID NO: 206 - DNA, 24 bp, synthetic construct
  source 1..24: mol_type = other DNA
caagcagaagacggcatacgagat

SEQ ID NO: 207 - DNA, 33 bp, synthetic construct; identical to SEQ ID NO: 7
  source 1..33: mol_type = other DNA

SEQ ID NO: 208 - DNA, 15 bp, synthetic construct
  source 1..15: mol_type = other DNA
cacccgagaattcca

SEQ ID NO: 209 - DNA, 72 bp, synthetic construct
  source 1..72: mol_type = other DNA
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_difference 18: note = a, c, t, g, unknown or other
  misc_difference 19: note = a, c, t, g, unknown or other
  misc_difference 20: note = a, c, t, g, unknown or other
  misc_difference 21: note = a, c, t, g, unknown or other
  misc_difference 22: note = a, c, t, g, unknown or other
  misc_difference 23: note = a, c, t, g, unknown or other
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
cccatataagaaannnnnnnnnnnnnnnnnnnnnnnnnnagatcggaagagcgtcgtgtagggaaagagtgt

SEQ ID NO: 210 - DNA, 29 bp, synthetic construct
  source 1..29: mol_type = other DNA
gtgtagatctcggtggtcgccgtatcatt

SEQ ID NO: 211 - DNA, 72 bp, synthetic construct; identical to SEQ ID NO: 204
  source 1..72: mol_type = other DNA
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
  misc_difference 42: note = a, c, t, g, unknown or other
  misc_difference 43: note = a, c, t, g, unknown or other
  misc_difference 44: note = a, c, t, g, unknown or other
  misc_difference 45: note = a, c, t, g, unknown or other
  misc_difference 46: note = a, c, t, g, unknown or other
  misc_difference 47: note = a, c, t, g, unknown or other
  misc_difference 48: note = a, c, t, g, unknown or other
  misc_difference 49: note = a, c, t, g, unknown or other
  misc_difference 50: note = a, c, t, g, unknown or other
  misc_difference 51: note = a, c, t, g, unknown or other
  misc_difference 52: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
  misc_difference 57: note = a, c, t, g, unknown or other
  misc_difference 58: note = a, c, t, g, unknown or other
  misc_difference 59: note = a, c, t, g, unknown or other

SEQ ID NO: 212 - DNA, 15 bp, synthetic construct
  source 1..15: mol_type = other DNA
tggaattctcgggtg

SEQ ID NO: 213 - DNA, 29 bp, synthetic construct
  source 1..29: mol_type = other DNA
aatgatacggcgaccaccgagatctacac

SEQ ID NO: 214 - DNA, 11 bp, synthetic construct
  source 1..11: mol_type = other DNA
acactctttcc

SEQ ID NO: 215 - DNA, 15 bp, synthetic construct
  source 1..15: mol_type = other DNA
cacccgagaattcca

SEQ ID NO: 216 - DNA, 72 bp, synthetic construct; identical to SEQ ID NO: 209
  source 1..72: mol_type = other DNA
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_difference 18: note = a, c, t, g, unknown or other
  misc_difference 19: note = a, c, t, g, unknown or other
  misc_difference 20: note = a, c, t, g, unknown or other
  misc_difference 21: note = a, c, t, g, unknown or other
  misc_difference 22: note = a, c, t, g, unknown or other
  misc_difference 23: note = a, c, t, g, unknown or other
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other

SEQ ID NO: 217 - DNA, 29 bp, synthetic construct
  source 1..29: mol_type = other DNA
aatgatacggcgaccaccgagatctacac

SEQ ID NO: 218 - DNA, 72 bp, synthetic construct; identical to SEQ ID NO: 204
  source 1..72: mol_type = other DNA
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
  misc_difference 42: note = a, c, t, g, unknown or other
  misc_difference 43: note = a, c, t, g, unknown or other
  misc_difference 44: note = a, c, t, g, unknown or other
  misc_difference 45: note = a, c, t, g, unknown or other
  misc_difference 46: note = a, c, t, g, unknown or other
  misc_difference 47: note = a, c, t, g, unknown or other
  misc_difference 48: note = a, c, t, g, unknown or other
  misc_difference 49: note = a, c, t, g, unknown or other
  misc_difference 50: note = a, c, t, g, unknown or other
  misc_difference 51: note = a, c, t, g, unknown or other
  misc_difference 52: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
  misc_difference 57: note = a, c, t, g, unknown or other
  misc_difference 58: note = a, c, t, g, unknown or other
  misc_difference 59: note = a, c, t, g, unknown or other

SEQ ID NO: 219 - DNA, 33 bp, synthetic construct
  source 1..33: mol_type = other DNA
tggaattctcgggtgccaaggaactccagtcac

SEQ ID NO: 220 - DNA, 24 bp, synthetic construct
  source 1..24: mol_type = other DNA
atctcgtatgccgtcttctgcttg

SEQ ID NO: 221 - DNA, 33 bp, synthetic construct
  source 1..33: mol_type = other DNA
agatcggaagagcgtcgtgtagggaaagagtgt

SEQ ID NO: 222 - DNA, 33 bp, synthetic construct; identical to SEQ ID NO: 219
  source 1..33: mol_type = other DNA

SEQ ID NO: 223 - DNA, 24 bp, synthetic construct
  source 1..24: mol_type = other DNA
caagcagaagacggcatacgagat

SEQ ID NO: 224 - DNA, 33 bp, synthetic construct; identical to SEQ ID NO: 7
  source 1..33: mol_type = other DNA

SEQ ID NO: 225 - DNA, 72 bp, synthetic construct; identical to SEQ ID NO: 209
  source 1..72: mol_type = other DNA
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_difference 18: note = a, c, t, g, unknown or other
  misc_difference 19: note = a, c, t, g, unknown or other
  misc_difference 20: note = a, c, t, g, unknown or other
  misc_difference 21: note = a, c, t, g, unknown or other
  misc_difference 22: note = a, c, t, g, unknown or other
  misc_difference 23: note = a, c, t, g, unknown or other
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other

SEQ ID NO: 226 - DNA, 29 bp, synthetic construct
  source 1..29: mol_type = other DNA
gtgtagatctcggtggtcgccgtatcatt

SEQ ID NO: 227 - DNA, 48 bp, synthetic construct
  source 1..48: mol_type = other DNA
aatgatacggcgaccaccgagatctacacgcttcctaacactctttcc

SEQ ID NO: 228 - DNA, 48 bp, synthetic construct
  source 1..48: mol_type = other DNA
aatgatacggcgaccaccgagatctacacttcttggcacactctttcc